(19) United States
(12) Patent Application Publication
(10) Pub. No.: US 2024/0002541 A1
(43) Pub. Date: Jan. 4, 2024

(54) METHODS AND MATERIALS FOR TREATING T CELL CANCERS

(71) Applicant: The Johns Hopkins University, Baltimore, MD (US)

(72) Inventors: Sarah DiNapoli, Baltimore, MD (US); Jacqueline Douglass, Baltimore, MD (US); Emily Han-Chung Hsiue, Baltimore, MD (US); Michael S. Hwang, Seattle, WA (US); Kenneth W. Kinzler, Baltimore, MD (US); Maximilian Konig, Baltimore, MD (US); Brian J. Mog, Baltimore, MD (US); Nickolas Papadopoulos, Towson, MD (US); Andrew M. Pardoll, Baltimore, MD (US); Suman Paul, Baltimore, MD (US); Alexander H. Pearlman, Baltimore, MD (US); Bert Vogelstein, Baltimore, MD (US); Shibin Zhou, Owings Mills, MD (US)

(21) Appl. No.: 18/039,403

(22) PCT Filed: Dec. 1, 2021
(86) PCT No.: PCT/US2021/061453

§ 371 Date: May 30, 2023

Related U.S. Application Data
(60) Provisional application No. 63/119,753, filed on Dec. 1, 2020.

(51) Int. Cl. C07K 16/46; A61P 35/00

(52) U.S. Cl. CPC C07K 16/468; A61P 35/00; C07K 2317/565; C07K 2317/31

(57) ABSTRACT

This document relates to methods and materials for treating T cell cancers. For example, a composition containing one or more bispecific molecules can be administered to a mammal having a T cell cancer to treat the mammal. For example, methods and materials for using one or more bispecific molecules to treat a mammal having a T cell cancer are provided.

### CROSS-REFERENCE TO RELATED APPLICATIONS

This application claims the benefit of U.S. Patent Application Ser. No. 63/119,753, filed on Dec. 1, 2020. The disclosure of the prior application is considered part of (and is incorporated by reference in) the disclosure of this application.

### STATEMENT REGARDING FEDERAL FUNDING

This invention was made with government support under grants CA009071, AR048522, CA006973, CA062924, GM007309 and CA230400 awarded by the National Institutes of Health. The government has certain rights in the invention.

### SEQUENCE LISTING

This document contains a Sequence Listing that has been submitted electronically as an ASCII text file named 44807-0384WO_SL_ST25.txt. The ASCII text file, created on Nov. 30, 2021, is 335 kilobytes in size. The material in the ASCII text file is hereby incorporated by reference in its entirety.

### BACKGROUND

### 1. Technical Field

This document relates to methods and materials for treating T cell cancers. For example, a composition containing one or more bispecific molecules can be administered to a mammal having a T cell cancer to treat the mammal. For example, this document provides methods and materials for using one or more bispecific molecules to treat a mammal having a T cell cancer.

### 2. Background Information

T cell cancers are a heterogeneous group of malignancies that comprises about 15% of non-Hodgkin's lymphomas (Swerdlow et al., Blood 127:2375-2390 (2016)) and 20% of acute lymphoblastic leukemias (ALL; Han et al., Cancer Causes & Control 19:841-858 (2008); and Dores et al., Blood 119:34-43 (2012). Outcomes of T cell lymphomas and relapsed T cell ALL (T-ALL) are worse than those for equivalent B cell malignancies, with an estimated 5-year survival of only 32% in T cell lymphomas (Weisenburger et al., Blood 117:3402-3408 (2011)) and 7% in relapsed T-ALL (Fielding et al., Blood 109:944-950 (2007)).

Malignant B or T cells do not express cell-surface antigens that are distinct from their non-cancerous counterparts. There are several targeted immunotherapeutic agents for B cell malignancies that target pan-B cell antigens such as CD19 or CD20, which is feasible because the associated normal B cell aplasia is clinically well tolerated. However, a similar strategy targeting pan-T cell antigens is not feasible because the resultant T cell depletion would lead to a clinically unacceptable level of immunosuppression.

### SUMMARY

This document provides methods and materials for treating T cell cancers. In some cases, this document provides bispecific molecules that can be used to treat T cell cancers. For example, a bispecific molecule that includes at least two antigen binding domains, where a first antigen binding domain (e.g., a first single-chain variable fragment (scFv)) can bind a T cell receptor β chain variable (TRBV) polypeptide and a second antigen binding domain (e.g., a second scFv) can bind a T cell co-receptor polypeptide, can be used to treat a mammal (e.g., a human) having a T cell cancer. In some cases, this document provides methods for treating T cell cancers. For example, one or more bispecific molecules provided herein (e.g., a composition containing one or more bispecific molecules provided herein) can be administered to a mammal having a T cell cancer to treat the mammal.

As demonstrated herein, T cell cancers can be treated by targeting specific subsets of T cell receptor (TCR) antigens. For example, bispecific antibodies targeting TRBV5-5 and CD3 can stimulate healthy T cells to specifically lyse TRBV5-5⁺ malignant T cell cells. Similarly, bispecific antibodies targeting TRBV12 and CD3 can stimulate healthy T cells to specifically lyse TRBV12⁺ malignant T cells. Also as demonstrated herein, bispecific antibodies targeting TRBV5-5 and CD3 and bispecific antibodies targeting TRBV12 and CD3 can preserve the majority of normal T cells within a mammal and can improve survival.

VDJ recombination, combined with allelic exclusion, results in expression of one of the 30 T cell receptor β chain variable (TRBV) polypeptides on the surface of each T cell, such that each TRBV is expressed on the surface of 1 to 5% of the total normal human peripheral blood T cells. In contrast, clonal T cell cancers express a single TRBV polypeptide. Having the ability to treat T cell cancers as described herein (e.g., by administering one or more bispecific molecules including a first antigen binding domain that can bind a TRBV polypeptide and a second antigen binding domain that can bind a T cell co-receptor polypeptide) provides a unique and unrealized opportunity to selectively deplete clonal T cell cancers while retaining the majority of the normal T cells (see, e.g., FIG. 1A). Additionally, bispecific molecules provided herein (e.g., a bispecific molecule including a first antigen binding domain that can bind a TRBV polypeptide and a second antigen binding domain that can bind a T cell co-receptor polypeptide) can be used as a cost-effective, off-the-shelf targeted therapeutic for T cell cancers.

In general, one aspect of this document features bispecific molecules including a first polypeptide comprising a first antigen binding domain that can bind a TRBV polypeptide, and a second polypeptide comprising a second antigen binding domain that can bind a T cell co-receptor polypeptide. The first polypeptide can be a single-chain variable fragment (scFv), an antigen-binding fragment (Fab), a F(ab′)2 fragment, or biologically active fragments thereof. The TRBV polypeptide can be a TRBV2 polypeptide, a TRBV3-1 polypeptide, a TRBV4-1 polypeptide, a TRBV4-2 polypeptide, a TRBV4-3 polypeptide, a TRBV5-1 polypeptide, a TRBV5-4 polypeptide, a TRBV5-5 polypeptide, a TRBV5-6 polypeptide, a TRBV5-8 polypeptide, a TRBV6-1 polypeptide, a TRBV6-2 polypeptide, a TRBV6-3 polypeptide, a TRBV6-4 polypeptide, a TRBV6-5 polypeptide, a TRBV6-6 polypeptide, a TRBV6-8 polypeptide, a TRBV6-9 polypeptide, a TRBV7-2 polypeptide, a TRBV7-3 polypeptide, a TRBV7-4 polypeptide, a TRBV7-6 polypeptide, a TRBV7-7 polypeptide, a TRBV7-8 polypeptide, a TRBV7-9 polypeptide, a TRBV9 polypeptide, a TRBV10-1 polypeptide, a TRBV10-2 polypeptide, a TRBV10-3 polypeptide, a TRBV11-1 polypeptide, a TRBV11-2 polypeptide, a TRBV11-3 polypeptide, a TRBV12-2 polypeptide, a TRBV12-3 polypeptide, a TRBV12-4 polypeptide, a TRBV12-5 polypeptide, a TRBV13 polypeptide, a TRBV14 polypeptide, a TRBV15 polypeptide, a TRBV16 polypeptide, a TRBV18 polypeptide, a TRBV19 polypeptide, a TRBV20-1 polypeptide, a TRBV24-1 polypeptide, a TRBV25-1 polypeptide, a TRBV27 TRBV28 polypeptide, a TRBV29-1 polypeptide, or a TRBV30 polypeptide. For example, the TRBV polypeptide can be a TRBV5-5 polypeptide. A first antigen binding domain that can bind to a TRBV5-5 polypeptide can include a light chain including a V_{L} CDR1 having an amino acid sequence set forth in SEQ ID NO:1, a V_{L} CDR2 having an amino acid sequence set forth in SEQ ID NO:2, and a V_{L} CDR3 having an amino acid sequence set forth in SEQ ID NO:3; and can include a heavy chain including a V_{H} CDR1 having an amino acid sequence set forth in SEQ ID NO:4, a V_{H} CDR2 having an amino acid sequence set forth in SEQ ID NO:5, and a V_{H} CDR3 having an amino acid sequence set forth in SEQ ID NO:6. In some cases, the light chain can include an amino acid sequence set forth in SEQ ID NO:7, and the heavy chain can include an amino acid sequence set forth in SEQ ID NO:8. In some cases, the light chain can include an amino acid sequence set forth in SEQ ID NO:38, and the heavy chain can include an amino acid sequence set forth in SEQ ID NO:39. For example, TRBV polypeptide can be TRBV12 polypeptide. A first antigen binding domain that can bind to a TRBV12 polypeptide can include a light chain including a V_{L} CDR1 having an amino acid sequence set forth in SEQ ID NO:9, a V_{L} CDR2 having an amino acid sequence set forth in SEQ ID NO:10, and a V_{L} CDR3 having an amino acid sequence set forth in SEQ ID NO:11; and can include a heavy chain including a V_{H} CDR1 having an amino acid sequence set forth in SEQ ID NO:12, a V_{H} CDR2 having an amino acid sequence set forth in SEQ ID NO:13, and a V_{H} CDR3 having an amino acid sequence set forth in SEQ ID NO:14. In some cases, the light chain can include an amino acid sequence set forth in SEQ ID NO: 15, and the heavy chain can include an amino acid sequence set forth in SEQ ID NO:16. In some cases, the light chain can include an amino acid sequence set forth in SEQ ID NO:40, and the heavy chain can include an amino acid sequence set forth in SEQ ID NO:41. The second polypeptide can be a scFv, an Fab, a F(ab′)2 fragment, or biologically active fragments thereof. The T cell co-receptor polypeptide can be a cluster of differentiation 3 (CD3) polypeptide or a T cell receptor polypeptide. A second antigen binding domain that can bind to a CD3 polypeptide can include a light chain including a V_{L} CDR1 having an amino acid sequence set forth in SEQ ID NO:17, a V_{L} CDR2 having an amino acid sequence set forth in SEQ ID NO:18, and a V_{L} CDR3 having an amino acid sequence set forth in SEQ ID NO:19; and can include a heavy chain including a V_{H} CDR1 having an amino acid sequence set forth in SEQ ID NO:20, a V_{H} CDR2 having an amino acid sequence set forth in SEQ ID NO:21, and a V_{H} CDR3 having an amino acid sequence set forth in SEQ ID NO:22. In some cases, the light chain can include an amino acid sequence set forth in SEQ ID NO:23, and the heavy chain can include an amino acid sequence set forth in SEQ ID NO:24.

In another aspect, this document features methods for treating a mammal having a T cell cancer. The methods can include, or consist essentially of, administering to a mammal having a T cell cancer a bispecific molecule including a first polypeptide having a first antigen binding domain that can bind a TRBV polypeptide, and a second polypeptide having a second antigen binding domain that can bind a T cell co-receptor polypeptide. The mammal can be a human. The T cell cancer can be a clonal T cell cancer. The T cell cancer can be an acute lymphoblastic leukemia (ALL), a peripheral T cell lymphomas (PTCL), an angioimmunoblastic T cell lymphomas (AITL), a T cell prolymphocytic leukemia (T-PLL), an adult T cell leukemia/lymphoma (ATLL), an nteropathy-associated T-cell lymphoma (EATL), a monomorphic epitheliotropic intestinal T-cell lymphoma (MEITL), a follicular T-cell lymphoma (FTCL), a nodal peripheral T-cell lymphoma (nodal PTCL), a cutaneous T cell lymphomas (CTCL), an anaplastic large cell lymphoma (ALCL), a T-cell large granular lymphocytic leukemia (T-LGL), an extra nodal NK/T-Cell lymphoma (NKTL), or a hepatosplenic T-cell lymphoma. The cancer cells within the mammal can be reduced by at least 95 percent. The method can be effective to improve survival of the mammal (e.g., can be effective to improve survival of the mammal by at least 37.5 percent).

In another aspect, this document features methods for treating a mammal having celiac disease. The methods can include, or consist essentially of, administering to a mammal having celiac disease a bispecific molecule including a first polypeptide having a first antigen binding domain that can bind a TRBV polypeptide and a second polypeptide having a second antigen binding domain that can bind a T cell co-receptor polypeptide. The mammal can be a human. The TRBV polypeptide can be a TRBV4 polypeptide, a TRBV6 polypeptide, a TRBV7 polypeptide, a TRBV9 polypeptide, a TRBV20, or a TRBV29 polypeptide, and the T cell co-receptor polypeptide can be a CD3 polypeptide. The TRBV polypeptide can be a TRBV6-1 polypeptide, a TRBV7-2 polypeptide, a TRBV9-1 polypeptide, a TRBV20-1 polypeptide, or a TRBV29-1 polypeptide, and the T cell co-receptor polypeptide can be a CD3 polypeptide.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1E: TRBV-specific BsAbs deplete cognate TRBV expressing T cells while preserving the majority of non-targeted T cells. FIG. 1A: Illustration depicting the proposed selective TRBV depletion strategy: Human T cells comprises 30 TRBV families; TRBV1: orange, TRBV5: red, TRBV12: cyan, TRBV20: green and TRBV30: purple cells. α-V12 binds TRBV12 expressing T cells leading to selective killing of the TRBV12 population while sparing the majority of the remaining non-TRBV12 T cells. FIG. 1B: α-V5, α-V12 and α-C1 BsAbs are composed of α-CD3 scFv (orange) linked with α-TRBV5-5 (red), α-TRBV12 (cyan) and α-TRBC1 (grey) scFvs respectively. Each scFv is composed of a variable heavy (V_{H}) and variable light (V_{L}) chain. FIG. 1C-FIG. 1E: 1×10⁶ normal human T cells were incubated with α-C1, α-V5 or α-V12 BsAbs (0.5 ng/ml) for 17 hours, followed by counting the number of surviving T cells and flow cytometric assessment of the TRBC and TRBV distribution in surviving T cells. Data are shown as the mean viable cell count from 5 different normal individuals. Also see FIG. 8.

FIGS. 2A-2I: TRBC1, TRBV5-5 or TRBV12 engagement activates T cells. FIG. 2A: Illustration depicting bidirectional T cell killing by α-C1, α-V5 and α-V12 BsAb. The conventional mechanism of action of α-C1, α-V5 and α-V12; involves crosslinking the T cell activating CD3 molecule (using α-CD3 scFv) on T cell #1 with TRBC1 (using α-C1), TRBV5-5 (using α-V5), or TRBV12 (using α-V12) on T cell #2 (e.g., a cancer cell), causing T cell #1 mediated killing of cell #2 (“1”). When target cell (cell #2) is also a T cell and can be activated by crosslinking with α-C1, α-V5 or α-V12, it will be able to function as an “effector” T cell and kill T cell #1 (“2”). FIG. 2B: Cartoons of α-CD3-CD19, α-C1-CD19, α-V5-CD19 and α-V12-CD19 BsAbs, composed of anti-CD19 scFv (black) linked to anti-CD3 (orange), anti-C1 (grey), anti-TRBV5 (red), and anti-TRBC1 (grey) scFvs. FIG. 2C: Illustration showing α-V12-CD19 BsAb crosslinking TRBV12 on a T cell with CD19 on an NALM6 B cell, causing T cell mediated NALM6 B cell killing. FIG. 2D and FIG. 2E: 5×10⁴ normal human T cells were incubated with 5×10⁴ wild-type (WT) or CD19 knock-out (CD19-KO) NALM6 B cells (expressing luciferase) with the indicated BsAbs (0.5 ng/ml) for 17 hours. IFNγ ELISA was used to assess normal human T cell activation (FIG. 2D) and luminescence was used to assess viable NALM6 B cells (FIG. 2E). In (FIG. 2D) and (FIG. 2E) Bars represent mean±standard error of mean using three different normal human T cells. ***P<0.001, ****P<0.0001, by one-way ANOVA with Dunnett's multiple comparison test. FIG. 2F-FIG. 2I: 5×10⁴ target NALM6 B cells (expressing luciferase) were incubated with 5×10⁴ normal human T cells, TRBV5 & TRBV12 depleted T cells (FIG. 2F) and (FIG. 2H), or TRBV5 (“TRBV5+”) enriched, or TRBV12 (“TRBV12+”) enriched T cells (FIG. 2G) and (FIG. 2I), along with indicated BsAbs (0.5 ng/ml) for 17 hours. IFNγ detection was used to assess normal human T cell activation (FIG. 2F) and (FIG. 2G). Luminescence was used to assess viable NALM6 B cells (FIG. 2H) and (FIG. 2I). In FIG. 2F and FIG. 2H bars represent mean±standard error of mean using three different normal human T cells. *P<0.05, **P<0.01. ***P<0.001. ns, not significant, by two-tailed paired t-test. In FIG. 2G and FIG. 2I bars represent mean±standard error of mean from three different human T cells. ****P<0.0001, by one-way ANOVA with Sidak multiple comparison test.

FIGS. 3A-3D: TRBV specific BsAbs induce T cell cytokine responses against cancer cells in vitro. FIG. 3A: 3.5×10⁴ normal human T cells were incubated with 3.5×10⁴ of the indicated target T cell cancer cell lines in the presence of α-C1 or α-V5 or α-V12 (0.5 ng/ml) for 17 hours. Luminescence was used to assess viable Jurkat (FIG. 3A) and HPB-ALL (FIG. 3B) cells. Bars represent mean±standard error of mean using three different normal human T cells. ****P<0.0001 by one-way ANOVA with Sidak multiple comparison test. FIG. 3B: 5×10⁴ normal human T cells or TRBV5- or TRBV12-depleted normal T cells were incubated with 5×10⁴ Jurkat cells or HPB-ALL cells in the presence of the indicated BsAbs (0.5 ng/ml) for 17 hours. T cell activation was then assessed by IFNγ ELISA. Bars represent mean±standard error of mean from three different human T cells. y, yes; n, no; **P<0.01. ***P<0.001. ns, not significant, by one-way ANOVA with Sidak multiple comparison test. (FIG. 3C) and (FIG. 3D) 5×10⁴ human T cells were incubated with 5×10⁴ HPB-ALL cells (FIG. 3C) or Jurkat T cells (FIG. 3D) in the presence of the indicated concentrations of α-V5 (FIG. 3C) or α-V12 (FIG. 3D) for 17 hours. T cell cytokine release was then measured with Luminex assay. The EC₅₀ (M) for each analyte is indicated in the corresponding graphs. Data shown as mean±standard error of mean from three different human T cells.

FIGS. 4A-4F: TRBV-specific BsAbs kill T cell cancer cells in vitro. FIG. 4A and FIG. 4B: 5×10⁴ human T cells were incubated with 5×10⁴ Jurkat cells (FIG. 4A) or HPB-ALL cells (FIG. 4B) in the presence of the indicated concentrations of α-V12 (FIG. 4A) α-V5 (FIG. 4B) for 17 hours. The Jurkat and HPB-ALL cells expressed luciferase. Luminescence was used to assess viable Jurkat and HPB-ALL cells. The EC₅₀ (M) for each BsAb is indicated in the corresponding graphs. Data shown as mean±standard error of mean using three different normal human T cells. FIG. 4C: 5×10⁴ normal human T cells were incubated with 5×10⁴ wild-type (WT) or TCR gene-disrupted (TCR-KO) Jurkat cells in the presence of the indicated BsAbs (0.5 ng/ml) for 17 hours. FIG. 4D: Identical experiments were performed with WT or with TCR-KO HPB-ALL cells. All Jurkat and HPB-ALL cells expressed GFP. Flow cytometry was then used to assess CD3 and GFP expression. In FIG. 4C and FIG. 4D, the numbers beside density plots indicate the percentage of surviving cells. FIG. 4E and FIG. 4F shows the aggregate data of percentage of tumor cells in each treatment condition using T cells from 3 different human donors. Bars represent mean±standard error of mean. ****P<; 0.0001. ns, not significant, by ANOVA with Sidak multiple comparison test. Also see FIG. 12.

FIGS. 5A-5F: TRBV-specific BsAb kills patient-derived T-ALL cells in vitro. FIG. 5A: Flow cytometric analysis of two T-ALL patient samples with circulating lymphoblasts expressing TRBV12. Numbers adjacent to the plots indicate the percentage of CD3+ cells that express TRBV12. FIG. 5B and FIG. 5C: 5×10⁴ normal human T cells were co-cultured with 5×10⁴ patient derived T-ALL target cells in the presence of the indicated BsAbs (0.5 ng/ml) for 17 hours. T cell activation was assessed by measurement of IFNγ in the supernatant (for patient 1 and patient 2) (FIG. 5B), or by flow cytometric analysis of indicated T cell activation and exhaustion markers (for patient 1) (FIG. 5C). Bars represent mean±standard error of mean from three technical replicates. ****P<0.001 by one-way ANOVA with Dunnett's multiple comparison test. FIG. 5D: Histogram of HLA-A3 stained normal human T cells and patient derived T-ALL malignant cells. FIG. 5E and (FIG. 5F: 5×10⁴ normal human T cells were co-cultured with 5×10⁴ patient-derived T-ALL target cells in the presence of α-CD19 or α-V12 BsAbs (0.5 ng/ml) for 17 hours. Flow cytometric analysis of HLA-A3 and CD3 was then performed. Numbers adjacent to the plots indicate the numbers of cells counted by flow cytometry in a representative experiment (FIG. 5E), with data from 3 technical replicates shown in (FIG. 5F). ****P≤0.0001 by one-way ANOVA with Tukey's multiple comparison test.

FIGS. 6A-6H: TRBV-specific BsAbs specifically kill cancer cells in vivo. FIG. 6A to FIG. 6C: NSG mice were intravenously injected with 5×10⁶ normal human T cells and 5×10⁶ WT or TCR-KO Jurkat cells. Identical experiments were performed with WT and TCR-KO HPB-ALL cells. All Jurkat and HPB-ALL cells expressed luciferase and GFP. Intraperitoneal pumps containing 100 μg of α-CD19, α-V12 or α-V5 BsAb were placed in the animals four days after cell injection, and BLI was performed on the indicated days. BLI data representative of one of two independent experiments (FIG. 6B) and (FIG. 6C). FIG. 6D: Combined radiance value from two independent experiments with a total of 11 NSG mice in each group was measured on the indicated days. **P<0.01, ****P<0.0001 by one-way ANOVA with Sidak's multiple comparison test. FIG. 6E and FIG. 6F: Flow cytometry on mouse blood collected on day 19 was used to detect circulating WT Jurkat or HPB-ALL cells (CD3+, GFP+, top right quadrant) or circulating TCR-KO Jurkat or HPB-ALL cells (CD3−, GFP+, bottom right quadrant) or circulating normal human T cells (CD3+, GFP−, top left quadrant) after the indicated treatments. Circulating cancer cell and T cell counts were assessed from six different NSG mice for each cancer cell type. Data shown as mean±standard error of mean, **P≤0.01, ****P<0.0001 by one-way ANOVA with Tukey's multiple comparison test. FIG. 6G and FIG. 6H: Kaplan-Meier survival curves of WT or TCR-KO Jurkat (FIG. 6G) or HPB-ALL (FIG. 6H) bearing NSG mice after various treatments. Median overall survival (OS) Jurkat WT/α-CD19=36 days, Jurkat WT/α-V12=73 days, Jurkat TCR-KO/α-V12=46 days. Jurkat WT/α-CD19 versus Jurkat WT/α-V12 hazard ratio (HR)=0.18, ****P<0.0001, log-rank (Mantel-Cox) test. Median OS HPB-ALL WT/α-CD19=40 days, HPB-ALL WT/α-V5=64 days, HPB-ALL TCR-KO/α-V5=33 days. HPB-ALL WT/α-CD19 versus HPB-ALL WT/α-V5, HR=0.19, ****P=0.0001, log-rank (Mantel-Cox) test. Survival data aggregated from 2 independent experiments.

FIGS. 7A-7E: α-V12 and α-V5 BsAb characteristics. FIG. 7A: Coomassie blue stain and western blot (using rabbit anti-6×His and HRP-conjugated anti-rabbit antibodies) of purified α-V12 and α-V5 BsAbs. FIG. 7B and FIG. 7C: Analytic chromatogram of purified α-V12 (FIG. 7B) or α-V5 (FIG. 7C) BsAb shown in black. Bovine serum albumin (BSA) chromatogram (control) shown in red. The retention time of each analyte is marked above the peak. FIG. 7D and FIG. 7E: Differential scanning fluorimetry analysis of α-V12 (FIG. 7D) or α-V5 (FIG. 7E) BsAb showing the negative derivative of relative fluorescence unit (“RFU”) vs. temperature (“Temp”). The melting temperatures correspond to the peak/maximums of the first derivative of the curve and are indicated in the corresponding graphs.

FIGS. 8A-8H: TRBV and TRBC-specific BsAb treatment of normal human T cells in vitro. FIG. 8A and FIG. 8B: 1×10⁶ normal human T cells were incubated with 0.5 ng/ml α-V5 or α-V12 BsAbs (FIG. 8A) or α-C1 (FIG. 8B), for 17 hours, followed by counting the number of surviving T cells and flow cytometric assessment of the TRBC and TRBV distribution in surviving T cells. Graph shows cell counts using T cells from 5 different normal individuals (FIG. 8A) and (FIG. 8B). FIG. 8C: TRBV5+ flow sorted T cells were stained with CellTrace Violet and mixed with TRBV5 depleted normal human T cells in 1:10 ratio. T cells were then incubated with α-V5 BsAb for 17 hours followed by flow cytometry. Similar experiment with TRBV12+ flow sorted T cells stained with CellTrace Violet, mixed with TRBV12 depleted normal human T cells (1:10 ratio) followed by incubation with α-V12 BsAb. Experiments repeated using 3 different normal human T cell donors. FIG. 8D and FIG. 8E: TRBC1 flow sorted T cells were stained with CellTrace Violet and mixed with TRBC1 depleted normal human T cells in 2:3 ratio. T cells were then incubated with α-C1 BsAb for 17 hours followed by flow cytometry analysis (FIG. 8D) and counting of the viable cells (FIG. 8E). FIG. 8F-FIG. 8H: 1×10⁶ normal human T cells (“normal”) or T cells depleted (“dep”) of TRBC1-expressing T cells (FIG. 8F) or TRBV5-expressing and TRBV12-expressing T cells (FIG. 7G) or TRBV5-enriched (TRBV5+) and TRBV12-enriched (TRBV12+) T cells (FIG. 8H) were incubated without (“no”) or with α-C1 (FIG. 8D), α-V5 or α-V12 BsAbs (FIG. 8E) (0.5 ng/ml) for 17 hours, followed by assessment of viable cells. Bars in FIG. 8F, FIG. 8G, and FIG. 8H represent mean±standard error of mean using three different normal human T cells. *P<0.05, **P<0.01, ***P<0.001 by two tailed paired t-test. ns, not significant.

FIGS. 9A-9E: TRBC1, TRBV5-5 or TRBV12 engagement activates T cells against NALM6 B cells. FIG. 9A: 2×10⁵ wild-type (WT), CD19 low or CD19 knock-out (CD19-KO) NALM6 B cells were stained with human anti-CD19 antibody followed by flow cytometry. Also shown are WT NALM6 B cells stained with isotype control antibody as control. FIG. 9B: 5×10⁴ normal human T cells were incubated with 5×10⁴ WT or CD19-KO NALM6 B cells with the indicated BsAbs (0.5 ng/ml) for 17 hours. IL-2, TNFα and IL-10 ELISA was used to assess normal human T cell activation. Bars represent mean standard error of mean using three different normal human T cells. *P<0.05, ***P<0.0001, by one-way ANOVA with Dunnett's multiple comparison test. FIG. 9C: 5×10⁴ normal human T cells were incubated in the presence or absence of 5×10⁴ WT NALM6 B cells with the indicated BsAbs (0.5 ng/ml) for 17 hours followed by flow cytometric analysis of indicated T cell activation and exhaustion markers. Bars represent mean±standard error of mean using three different normal human T cells. Histograms below individual bar graphs show data from one human T cell donor. *P<0.05, ***P<0.0001, by one-way ANOVA with Dunnett's multiple comparison test. FIG. 9D and FIG. 9E: 5×10⁴ normal human T cells were incubated in the presence of 5×10⁴ WT or CD19 low NALM6 B cells (expressing luciferase) with the indicated BsAbs (0.5 ng/ml) for 17 hours. IFNγ ELISA was used to assess normal human T cell activation (FIG. 9D) and luminescence was used to assess viable NALM6 B cells (FIG. 9E). In FIG. 9D and FIG. 9E bars represent mean±standard error of mean using three different human T cells. ns, not significant, by two tailed unpaired t test.

FIGS. 10A-10D: α-C1 BsAb kills both TRBC1+ and TRBC2+ expressing T cells. FIG. 10A: Illustration of the mechanism of α-C1 killing of TRBC2+ HPB-ALL cells. α-C1 crosslinks TRBC1+ T cells (“T”) (using α-TRBC1 scFv) with HPB-ALL cells (“H”) (using α-CD3 scFv) causing HPB-ALL cell death. FIG. 10B: Normal human T cells (“undepleted”) or TRBC1 depleted T cells were stained with TRBC1 antibody and assessed by flow cytometry. Histograms representative of 3 independent experiments are shown. FIG. 10C and FIG. 10D: 5×10⁴ normal human undepleted T cells or TRBC1 depleted (“TRBC1 dep”) T cells were incubated with 5×10⁴ Jurkat cells (TRBC1+) or HPB-ALL cells (TRBC2+) with or without α-C1 BsAb (0.5 ng/ml) for 17 hours. T cell activation was assessed by measurement of IFNγ in supernatant (FIG. 10C), and killing of cancer cells was assessed by flow cytometry (FIG. 10D). Jurkat (“J”) and HPB-ALL (“H”) cells are GFP+ and CD3+, and normal human T cells (“T”) are GFP− and CD3+. The numbers inside the plots indicate the percentage of surviving cells. Illustrations beside flow plots demonstrate mechanisms of α-C1 BsAb mediated killing of target Jurkat and HPB-ALL cells. Undepleted T cells can kill Jurkat cells by two mechanisms (shown as “1” and “2”). Undepleted T cells can kill HPB-ALL by only one mechanism (shown as “2”). TRBC1 depleted T cells (consisting of only TRBC2 T cells) can continue to kill Jurkat cells using mechanism “1”, while TRBC1 depleted T cells (or TRBC2 T cells) cannot kill HPB-ALL cells (FIG. 10D). In FIG. 10C bars represent mean±standard error of mean from three technical replicates. ***P<0.001. ns, not significant, by one-way ANOVA with Tukey's multiple comparison test.

FIGS. 11A-11B: Cell-surface CD3 and TRBV expression in T cell cancer cell lines. FIG. 11A and FIG. 11B: 2×10⁵ human T cell cancer-derived cell lines were stained with isotype control antibody or human anti-CD3 (FIG. 11A) or anti-TRBV5-5 and anti-TRBV12-specific antibodies (FIG. 11B), followed by flow cytometry. Data are representative of two independent experiments.

FIGS. 12A-12F: TRBV specific BsAbs activate healthy T cells to kill T cell cancer cells in vitro. 5×10⁴ normal human T cells from 2 additional normal human individuals (donor 2 and donor 3) were incubated with 5×10⁴ WT or TCR-KO Jurkat cells in the presence of indicated BsAbs (0.5 ng/ml) for 17 hours. Identical experiments were performed with WT or with TCR-KO HPB-ALL cells. FIG. 12A: All Jurkat and HPB-ALL cells expressed GFP. Flow cytometry was then used to assess CD3 and GFP expression. The numbers beside density plots indicate the percentage of surviving cells. FIG. 12B and FIG. 12C: 5×10⁴ normal human T cells were incubated with 5×10⁴ WT Jurkat (FIG. 12B) or WT HPB-ALL cells (FIG. 12C) with the indicated BsAbs (0.5 ng/ml) for 17 hours followed by flow cytometric analysis of the indicated T cell activation and exhaustion markers. Bars represent mean±standard error of mean using three different normal human T cells. ***P<0.0001, by one-way ANOVA with Dunnett's multiple comparison test. FIG. 12D: Normal human T cells or CD4 depleted T cells were stained with anti-human CD4 or CD8 antibodies and assessed by flow cytometry. FIG. 12E: 5×10⁴ normal human T cells or CD4-depleted T cells were incubated with 5×10⁴ Jurkat cells or HPB-ALL cells (expressing luciferase) in the presence of α-CD19, α-V12 BsAb or α-V5 (0.5 ng/ml) for 17 hours. Luminescence was used to assess viable Jurkat or HPB-ALL cells. Bars represent mean±standard error of mean using T cells from three different normal human individuals. ***P<0.0001, by two-tailed unpaired t test. FIG. 12F: α-V12 or αV-5 BsAbs incubated with human serum until indicated time points. BsAb activity was assessed by co-culture of 5×10⁴ normal human T cells, with 5×10⁴ Jurkat cells or HPB-ALL cells. Graphs show the percentage of viable Jurkat or HPB-ALL cells after incubation with T cells from three different normal human individuals.

FIGS. 13A-13B: TCRβ sequencing to assess α-V12 and α-V5 and targeting specificity. FIG. 13A and FIG. 13B: 1×10⁵ normal human T cells and 1×10⁵ Jurkat cells (FIG. 13A) or 1×10⁵ HPB-ALL cells (FIG. 13B) were incubated with α-CD19 (green circle) or α-V12 (blue squares) (FIG. 13A) or α-V5 (orange squares) (FIG. 13B) BsAbs (0.5 ng/ml) for 17 hours. RNA was purified from the cells and TCRβ sequencing was used to identify TRBV families. Numbers represent mean±standard error of mean of percentage of TRBV distribution from three technical replicates. Black arrows point to the TRBV12-3 signal from Jurkat cells and the TRBV5-5 signal from HPB-ALL cells. Numbers beside arrows indicate TRBV percentages. Data are representative of two independent experiments.

FIGS. 14A-14C: TRBV5 family sequence alignment and structural analysis. FIG. 14A and FIG. 14B: TRBV5 family phylogram (FIG. 14A) and TRBV5 family sequence alignment (FIG. 14B). Amino acid residues in bold are shared across all TRBV5 family members. Positions 20, 81 and 101 are highlighted in green and demonstrate residues common to TRBV5-5 and 5-6, but distinct in other TRBV5 members. FIG. 14C: TRBV5-1 structure (cyan) with inset showing amino acid positions 81 (D in TRBV5-5/5-6 versus G in 5-1 or P in 5-4/5-8) and 101 (L in TRBV5-5/5-6 versus E in TRBV5-1/5-4/5-8) as sticks. TRBV5-5/5-6 shown in yellow; TRBV5-1/5-4/5-8 shown in dark blue.

FIGS. 15A-15I: TRBV-specific BsAbs activate human T cells to specifically kill T cell cancers in vivo at low E:T ratio. FIG. 15A: Intraperitoneal pumps containing 100 μg of α-V12 or α-V5 BsAb were placed in 3 NSG mice on day 0, followed by daily mouse blood collection and detection of indicated BsAbs. FIG. 15B and FIG. 15C: NSG mice were intravenously injected with 0.5×10⁶ normal human T cells and 2.5×10⁶ WT Jurkat cells or WT HPB-ALL cells. All Jurkat and HPB-ALL cells expressed luciferase and GFP. Intraperitoneal pumps containing 100 μg of α-CD19, α-V12 or α-V5 BsAb were placed in the animals four days after cell injection. Mouse blood collection and BLI was performed on the indicated days. FIG. 15D and FIG. 15E: Radiance values in each group were measured on the indicated days. *P<0.05, **P<0.01, by unpaired two-tailed t test. FIG. 15F and FIG. 15G: IFNγ and TNFα ELISA from mouse serum collected on day 3 and day 6. FIG. 15H and FIG. 15I: Flow cytometry on mouse blood collected on day 3 and day 6 to detect activation or exhaustion markers on circulating normal human T cells. Data shown as mean±standard error of mean, ***P<0.001 by one-way ANOVA with Sidak's multiple comparison test.

### DETAILED DESCRIPTION

This document provides methods and materials for treating T cell cancers. In some cases, this document provides bispecific molecules that can be used to treat T cell cancers. For example, this document provides bispecific molecules that include at least two antigen binding domains where a first antigen binding domain (e.g., a first scFv) can bind a TRBV polypeptide and a second antigen binding domain (e.g., a second scFv) can bind a T cell co-receptor polypeptide) can be used to treat a mammal (e.g., a human) having a T cell cancer. This document also provides methods for treating T cell cancers. For example, one or more bispecific molecules provided herein (e.g., a composition containing one or more bispecific molecules provided herein) can be administered to a mammal having a T cell cancer to treat the mammal. In some cases, a bispecific molecule provided herein (e.g., a bispecific molecule including a first antigen binding domain that can bind a TRBV polypeptide and a second antigen binding domain that can bind a T cell co-receptor polypeptide) can activate T cells within a mammal to target (e.g., target and destroy) T cells expressing a TRBV polypeptide that can be targeted by the bispecific molecule. For example, a T cell expressing a T cell co-receptor polypeptide that can be targeted by a bispecific molecule provided herein can be activated to target (e.g., target and destroy) T cells (e.g., cancerous T cells) expressing a TRBV polypeptide that can be targeted by the bispecific molecule.

Any appropriate mammal (e.g., a mammal having a T cell cancer) can be treated as described herein. For example, humans, non-human primates (e.g., monkeys), horses, bovine species, porcine species, dogs, cats, mice, and rats can be treated as described herein. In some cases, a human having a T cell cancer can be administered one or more bispecific molecules provided herein (e.g., bispecific molecules including a first antigen binding domain that can bind a TRBV polypeptide and a second antigen binding domain that can bind a T cell co-receptor polypeptide).

The materials and methods described herein can be used to treat a mammal (e.g., a human) having any type of T cell cancer. In some cases, a T cell cancer treated as described herein can include one or more solid tumors. In some cases, a T cell cancer treated as described herein can be a blood cancer. In some cases, a T cell cancer treated as described herein can be a primary cancer. In some cases, a T cell cancer treated as described herein can be a metastatic cancer. In some cases, a T cell cancer treated as described herein can be a refractory cancer. In some cases, a T cell cancer treated as described herein can be a non-Hodgkin's lymphoma. In some cases, a T cell cancer treated as described herein can be a Hodgkin's lymphoma. Examples of T cell cancers that can be treated as described herein include, without limitation, ALL, PTCL, AITL, T-PLL, ATLL, EATL, MEITL, FTCL, nodal PTCL, CTCL, ALCL, T-LGL, NKTL, and hepatosplenic T-cell lymphoma.

In some cases, the materials and methods provided herein can be used to reduce or eliminate the number of cancer cells present within a mammal (e.g., a human) having a T cell cancer. For example, a mammal in need thereof (e.g., a mammal having a T cell cancer) can be administered one or more bispecific molecules provided herein (e.g., bispecific molecules including a first antigen binding domain that can bind a TRBV polypeptide and a second antigen binding domain that can bind a T cell co-receptor polypeptide) to reduce or eliminate the number of cancer cells present within the mammal. For example, the materials and methods described herein can be used to reduce the number of cancer cells present within a mammal having cancer by, for example, 10, 20, 30, 40, 50, 60, 70, 80, 90, 95, or more percent. For example, the materials and methods described herein can be used to reduce the size (e.g., volume) of one or more tumors present within a mammal having cancer by, for example, 10, 20, 30, 40, 50, 60, 70, 80, 90, 95, or more percent. In some cases, the number of cancer cells present within a mammal being treated can be monitored. Any appropriate method can be used to determine whether or not the number of cancer cells present within a mammal is reduced. For example, imaging techniques can be used to assess the number of cancer cells present within a mammal.

In some cases, the materials and methods provided herein can be used to improve survival of a mammal (e.g., a human) having a T cell cancer. For example, a mammal in need thereof (e.g., a mammal having a T cell cancer) can be administered one or more bispecific molecules provided herein (e.g., bispecific molecules including a first antigen binding domain that can bind a TRBV polypeptide and a second antigen binding domain that can bind a T cell co-receptor polypeptide) to improve survival of the mammal. For example, the materials and methods described herein can be used to improve the survival of a mammal having cancer by, for example, 10, 20, 30, 40, 50, 60, 70, 80, 90, 95, or more percent. For example, the materials and methods described herein can be used to improve the survival of a mammal having cancer by, for example, at least 6 months (e.g., about 6 months, about 8 months, about 10 months, about 1 year, about 1.5 years, about 2 years, about 2.5 years, about 3 years, about 4 years, about 5 years, or more).

In some cases, when a mammal in need thereof (e.g., a mammal having a T cell cancer) is administered one or more bispecific molecules provided herein (e.g., bispecific molecules including a first antigen binding domain that can bind a TRBV polypeptide and a second antigen binding domain that can bind a T cell co-receptor polypeptide), the majority of normal T cells within the mammal can be preserved. For example, the materials and methods described herein can be used to treat mammal having a T cell cancer as described herein while preserving, for example, 50, 60, 70, 80, 90, 95, or more percent of normal (e.g., non-cancerous) T cells within the mammal. In some cases, from about 75 percent to about 100 percent (e.g., from about 75 percent to about 99 percent, from about 75 percent to about 95 percent, from about 75 percent to about 93 percent, from about 75 percent to about 90 percent, from about 75 percent to about 85 percent, from about 80 percent to about 100 percent, from about 85 percent to about 100 percent, from about 90 percent to about 100 percent, or from about 95 percent to about 100 percent) of normal (e.g., non-cancerous) T cells within a mammal can be preserved when the mammal is administered one or more bispecific molecules provided herein.

In some cases, the methods described herein also can include identifying a mammal as having a T cell cancer. Examples of methods for identifying a mammal as having a T cell cancer include, without limitation, physical examination, laboratory tests (e.g., blood and/or urine), biopsy, imaging tests (e.g., X-ray, PET/CT, MRI, and/or ultrasound), nuclear medicine scans (e.g., bone scans), endoscopy, and/or genetic tests. Once identified as having a T cell cancer, a mammal can be administered or instructed to self-administer one or more bispecific molecules provided herein (e.g., bispecific molecules including a first antigen binding domain that can bind a TRBV polypeptide and a second antigen binding domain that can bind a T cell co-receptor polypeptide).

Any appropriate bispecific molecule can be administered to a mammal (e.g., a human) as described herein. In some cases, a bispecific molecule can include at least two (e.g., two, three, or four) antigen binding domains, where a first antigen binding domain (e.g., a first scFv) can bind a TRBV polypeptide and a second antigen binding domain (e.g., a second scFv) can bind a T cell co-receptor polypeptide. In some cases, a bispecific molecule provided herein can include a first antigen binding domain that can bind a TRBV polypeptide and a second antigen binding domain that can bind a T cell co-receptor polypeptide.

A first antigen binding domain in a bispecific molecule provided herein (e.g., a bispecific molecule including a first antigen binding domain that can bind a TRBV polypeptide and a second antigen binding domain that can bind a T cell co-receptor polypeptide) can be any appropriate type of antigen binding domain. In some cases, a first antigen binding domain that can be used in a bispecific molecule provided herein can include a variable region of an immunoglobulin light chain (a VL) and a variable region of an immunoglobulin heavy chain (VH). For example, a first antigen binding domain that can be used in a bispecific molecule provided herein can include a first complementarity determining region (CDR) from an immunoglobulin light chain (a V_{L} CDR1), a second CDR from an immunoglobulin light chain (a V_{L} CDR2), and a third CDR an immunoglobulin light chain (a V_{L} CDR3), a first CDR from an immunoglobulin heavy chain (a V_{H} CDR1), a second CDR from an immunoglobulin heavy chain (a V_{H} CDR2), and a third CDR an immunoglobulin heavy chain (a V_{H} CDR2). Examples of antigen binding domains that can be used as a can be used as a first antigen binding domain in a bispecific molecule provided herein include, without limitation, single-chain variable fragment (scFv), an antigen-binding fragment (Fab), a F(ab′)2 fragment, and biologically active fragments thereof (e.g., a fragment that retains the ability to bind the target molecule such as a TRBV polypeptide). In some cases, an antigen binding domain that can be used as a first antigen binding domain in a bispecific molecule provided herein can be a scFv.

A first antigen binding domain in a bispecific molecule provided herein (e.g., a bispecific molecule including a first antigen binding domain that can bind a TRBV polypeptide and a second antigen binding domain that can bind a T cell co-receptor polypeptide) can bind any appropriate TRBV Examples of TRBVs that can be targeted by a first antigen binding domain in a bispecific molecule provided herein include, without limitation, TRBV2 polypeptides, TRBV3-1 polypeptides, TRBV4-1 polypeptides, TRBV4-2 polypeptides, TRBV4-3 polypeptides, TRBV5-1 polypeptides, TRBV5-4 polypeptides, TRBV5-5 polypeptides, TRBV5-6 polypeptides, TRBV5-8 polypeptides, TRBV6-1 polypeptides, TRBV6-2 polypeptides, TRBV6-3 polypeptides, TRBV6-4 polypeptides, TRBV6-5 polypeptides, TRBV6-6 polypeptides, TRBV6-8 polypeptides, TRBV6-9 polypeptides, TRBV7-2 polypeptides, TRBV7-3 polypeptides, TRBV7-4 polypeptides, TRBV7-6 polypeptides, TRBV7-7 polypeptides, TRBV7-8 polypeptides, TRBV7-9 polypeptides, TRBV9 polypeptides, TRBV10-1 polypeptides, TRBV10-2 polypeptides, TRBV10-3 polypeptides, TRBV11-1 polypeptides, TRBV11-2 polypeptides, TRBV11-3 polypeptides, TRBV12-2 polypeptides, TRBV12-3 polypeptides, TRBV12-4 polypeptides, TRBV12-5 polypeptides, TRBV13 polypeptides, TRBV14 polypeptides, TRBV15 polypeptides, TRBV16 polypeptides, TRBV18 polypeptides, TRBV19 polypeptides, TRBV20-1 polypeptides, TRBV24-1 polypeptides, TRBV25-1 polypeptides, TRBV27 TRBV28 polypeptides, TRBV29-1 polypeptides, and TRBV30 polypeptides. In some cases, a first antigen binding domain that binds a TRBV is specific for that TRBV For example, a first antigen binding domain that binds a TRBV can bind to that TRBV with an affinity having a dissociation constant (K_{D}) of from about 2 nM to about 30 nM (e.g., from about 2 nM to about 25 nM, from about 2 nM to about 20 nM, from about 2 nM to about 15 nM, from about 2 nM to about 10 nM, from about 2 nM to about 5 nM, from about 5 nM to about 30 nM, from about 10 nM to about 30 nM, from about 15 nM to about 30 nM, from about 20 nM to about 30 nM, from about 25 nM to about 30 nM, from about 2.6 nM to about 25.2 nM, from about 5 nM to about 20 nM, from about 10 nM to about 15 nM, from about 5 nM to about 10 nM, from about 15 nM to about 20 nM, or from about 20 nM to about 25 nM). In some cases, a first antigen binding domain that specifically binds a TRBV does not bind (or does not substantially bind) a different TRBV. In some cases, a first antigen binding domain in a bispecific molecule provided herein can be as described elsewhere (see, e.g., Wang et al., Nat. Genet., 47, 1426-1434 (2015); and de Masson et al., Sci. Transl. Med., 10, (2018)).

In some cases, a first antigen binding domain that can be used in a bispecific molecule provided herein can bind to a TRBV5-5 polypeptide. For example, an antigen binding domain that can bind to a TRBV5-5 polypeptide can include each of the CDRs set forth below:

| | Sequence | SEQ ID NO |
|---|---|---|
| V_{L} CDR1 | CSASQGISNYLN | 1 |
| V_{L} CDR2 | TSSLHSGV | 2 |
| V_{L} CDR3 | QQYSKLPRT | 3 |
| V_{H} CDR1 | AYGVN | 4 |
| V_{H} CDR2 | WGDGNTDYNSALK | 5 |
| V_{H} CDR3 | ATLYAMDY | 6 |

In some cases, an antigen binding domain that can bind to a TRBV5-5 polypeptide can include a light chain having a V_{L} CDR1 including the amino acid sequence set forth in SEQ ID NO:1, a V_{L} CDR2 including the amino acid sequence set forth in SEQ ID NO:2, and a V_{L} CDR3 including the amino acid sequence set forth in SEQ ID NO:3. For example, an antigen binding domain that can bind to a TRBV5-5 polypeptide can include a light chain including the amino acid sequence set forth in SEQ ID NO:7. For example, an antigen binding domain that can bind to a TRBV5-5 polypeptide can include a light chain including the amino acid sequence set forth in SEQ ID NO:38. In some cases, an antigen binding domain that can bind to a TRBV5-5 polypeptide can include a heavy chain having a V_{H} CDR1 including the amino acid sequence set forth in SEQ ID NO:4, a V_{H} CDR2 including the amino acid sequence set forth in SEQ ID NO:5, and a V_{H} CDR3 including the amino acid sequence set forth in SEQ ID NO:6. For example, an antigen binding domain that can bind to a TRBV5-5 polypeptide can include a heavy chain including the amino acid sequence set forth in SEQ ID NO:8. For example, an antigen binding domain that can bind to a TRBV5-5 polypeptide can include a heavy chain including the amino acid sequence set forth in SEQ ID NO:39. In some cases, an antigen binding domain that can bind to a TRBV5-5 polypeptide can include a light chain having a V_{L} CDR1 including the amino acid sequence set forth in SEQ ID NO:1, a V_{L} CDR2 including the amino acid sequence set forth in SEQ ID NO:2, and a V_{L} CDR3 including the amino acid sequence set forth in SEQ ID NO:3, and can include a heavy chain having a V_{H} CDR1 including the amino acid sequence set forth in SEQ ID NO:4, a V_{H} CDR2 including the amino acid sequence set forth in SEQ ID NO:5, and a V_{H} CDR3 including the amino acid sequence set forth in SEQ ID NO:6. For example, an antigen binding domain that can bind to a TRBV5-5 polypeptide can include a light chain including the amino acid sequence set forth in SEQ ID NO:7 and can include a heavy chain including the amino acid sequence set forth in SEQ ID NO:8. For example, an antigen binding domain that can bind to a TRBV5-5 polypeptide can include a light chain including the amino acid sequence set forth in SEQ ID NO:38 and can include a heavy chain including the amino acid sequence set forth in SEQ ID NO:39.

In some cases, a first antigen binding domain that can be used in a bispecific molecule provided herein can bind to a TRBV12 polypeptide. For example, an antigen binding domain that can bind to a TRBV12 polypeptide can include each of the CDRs set forth below:

| | Sequence | SEQ ID NO |
|---|---|---|
| V_{L} CDR1 | CRASSSVNYIYW | 9 |
| V_{L} CDR2 | YTSNLAPGVP | 10 |
| V_{L} CDR3 | QQFTSSPFT | 11 |
| V_{H} CDR1 | NFGMH | 12 |
| V_{H} CDR2 | YISSGSSTIYYADTLKG | 13 |
| V_{H} CDR3 | RGEGAMDY | 14 |

20

In some cases, an antigen binding domain that can bind to a TRBV12 polypeptide can include a light chain having a V_{L} CDR1 including the amino acid sequence set forth in SEQ ID NO:9, a V_{L} CDR2 including the amino acid sequence set forth in SEQ ID NO:10, and a V_{L} CDR3 including the amino acid sequence set forth in SEQ ID NO: 11. For example, an antigen binding domain that can bind to a TRBV12 polypeptide can include a light chain including the amino acid sequence set forth in SEQ ID NO:15. For example, an antigen binding domain that can bind to a TRBV12 polypeptide can include a light chain including the amino acid sequence set forth in SEQ ID NO:40. In some cases, an antigen binding domain that can bind to a TRBV12 polypeptide can include a heavy chain having a V_{H} CDR1 including the amino acid sequence set forth in SEQ ID NO:12, a V_{H} CDR2 including the amino acid sequence set forth in SEQ ID NO:13, and a V_{H} CDR3 including the amino acid sequence set forth in SEQ ID NO:14. For example, an antigen binding domain that can bind to a TRBV12 polypeptide can include a heavy chain including the amino acid sequence set forth in SEQ ID NO:16. For example, an antigen binding domain that can bind to a TRBV12 polypeptide can include a heavy chain including the amino acid sequence set forth in SEQ ID NO:41. In some cases, an antigen binding domain that can bind to a TRBV12 polypeptide can include a light chain having a V_{L} CDR1 including the amino acid sequence set forth in SEQ ID NO:9, a V_{L} CDR2 including the amino acid sequence set forth in SEQ ID NO: 10, and a V_{L} CDR3 including the amino acid sequence set forth in SEQ ID NO: 11, and can include a heavy chain having a V_{H} CDR1 including the amino acid sequence set forth in SEQ ID NO:12, a V_{H} CDR2 including the amino acid sequence set forth in SEQ ID NO:13, and a V_{H} CDR3 including the amino acid sequence set forth in SEQ ID NO:14. For example, an antigen binding domain that can bind to a TRBV12 polypeptide can include a light chain including the amino acid sequence set forth in SEQ ID NO:15 and can include a heavy chain including the amino acid sequence set forth in SEQ ID NO:16. For example, an antigen binding domain that can bind to a TRBV5-5 polypeptide can include a light chain including the amino acid sequence set forth in SEQ ID NO:40 and can include a heavy chain including the amino acid sequence set forth in SEQ ID NO:41.

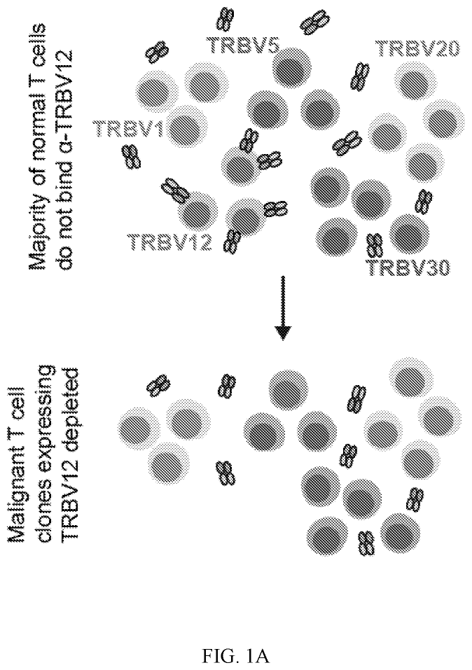

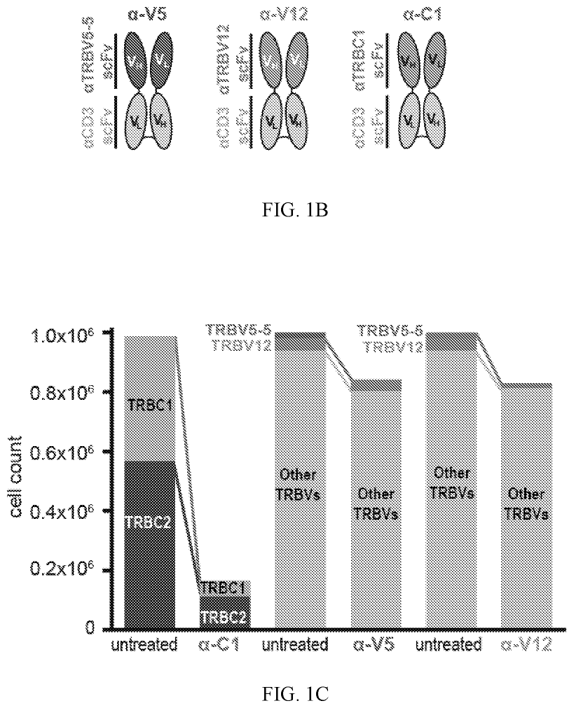

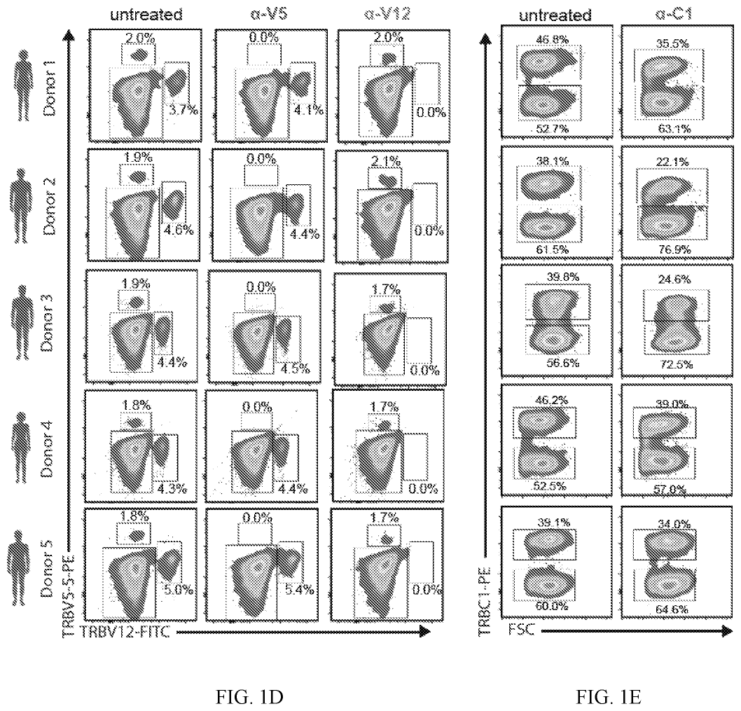

In some cases, a first antigen binding domain in a bispecific molecule provided herein (e.g., a bispecific molecule including a first antigen binding domain that can bind a TRBV polypeptide and a second antigen binding domain that can bind a T cell co-receptor polypeptide) can be as described elsewhere (see, e.g., Beta Mark TCR Vbeta Repertoire Kit, 25 Tests, RUO, Package insert. Beckman Coulter Life Sciences, Technical Document (2009); and U.S. Pat. No. 5,861,155 at, for example, FIG. 1).

A second antigen binding domain in a bispecific molecule provided herein (e.g., a bispecific molecule including a first antigen binding domain that can bind a TRBV polypeptide and a second antigen binding domain that can bind a T cell co-receptor polypeptide) can be any appropriate type of antigen binding domain. In some cases, a second antigen binding domain that can be used in a bispecific molecule provided herein can include a variable region of an immunoglobulin light chain (a VL) and a variable region of an immunoglobulin heavy chain (VH). For example, a second antigen binding domain that can be used in a bispecific molecule provided herein can include a first complementarity determining region (CDR) from an immunoglobulin light chain (a V_{L} CDR1), a second CDR from an immunoglobulin light chain (a V_{L} CDR2), and a third CDR an immunoglobulin light chain (a V_{L} CDR3), a first CDR from an immunoglobulin heavy chain (a V_{H} CDR1), a second CDR from an immunoglobulin heavy chain (a V_{H} CDR2), and a third CDR an immunoglobulin heavy chain (a V_{H} CDR2). Examples of antigen binding domains that can be used as a can be used as a second antigen binding domain in a bispecific molecule provided herein include, without limitation, scFv, a Fab, a F(ab′)2 fragment, and biologically active fragments thereof (e.g., a fragment that retains the ability to bind the target molecule such as a T cell co-receptor polypeptide). In some cases, an antigen binding domain that can be used as a second antigen binding domain in a bispecific molecule provided herein can be a scFv.

A second antigen binding domain in a bispecific molecule provided herein (e.g., a bispecific molecule including a first antigen binding domain that can bind a TRBV polypeptide and a second antigen binding domain that can bind a T cell co-receptor polypeptide) can bind any appropriate T cell co-receptor polypeptide. Examples of T cell co-receptor polypeptides that can be targeted by a second antigen binding domain in a bispecific molecule provided herein include, without limitation, CD3 polypeptides and T cell receptor polypeptides.

In some cases, a second antigen binding domain that can be used in a bispecific molecule provided herein can bind to a CD3 polypeptide. For example, an antigen binding domain that can bind to a CD3 polypeptide can include one of each of the CDRs set forth below:

| | Sequence | SEQ ID NO |
|---|---|---|
| V_{L} CDR1 | RASQDIRNYLN | 17 |
| V_{L} CDR1 | RASSSVSYMN | 44 |
| V_{L} CDR1 | SASSSVSYMN | 45 |
| V_{L} CDR1 | RSSTGAVTTSNYAN | 46 |
| V_{L} CDR1 | RASQSVSYMN | 47 |
| V_{L} CDR2 | (Y)YTSRLHS | 18 |
| | (with the first | |
| | Y being optional) | |
| V_{L} CDR2 | DTSKVAS | 48 |
| V_{L} CDR2 | DTSKLAS | 49 |
| V_{L} CDR2 | GTNKRAP | 50 |
| V_{L} CDR3 | QQGNTLPWT | 19 |
| V_{L} CDR3 | QQWSSNPLT | 51 |
| V_{L} CDR3 | QQWSSNPFT | 52 |
| V_{L} CDR3 | ALWYSNLWV | 53 |
| V_{H} CDR1 | GYTMN | 20 |
| V_{H} CDR1 | RYTMH | 54 |
| V_{H} CDR1 | TYAMN | 55 |
| V_{H} CDR2 | LINPYKGVSTYNQKFKD | 21 |
| V_{H} CDR2 | YINPSRGYTNYNQKFK | 56 |
| V_{H} CDR2 | RIRSKYNNYATYYADSVKD | 57 |
| V_{H} CDR2 | YINPSRGYTNYADSVKG | 58 |
| V_{H} CDR3 | SGYYGDSDWYFDV | 22 |
| V_{H} CDR3 | YYDDHYCLDY | 59 |
| V_{H} CDR3 | HGNFGNSYVSWFAY | 60 |

In some cases, an antigen binding domain that can bind to a CD3 polypeptide can include a light chain having a V_{L} CDR1 including the amino acid sequence set forth in SEQ ID NO: 17, a V_{L} CDR2 including the amino acid sequence set forth in SEQ ID NO:18, and a V_{L} CDR3 including the amino acid sequence set forth in SEQ ID NO:19. For example, an antigen binding domain that can bind to a CD3 polypeptide can include a light chain including the amino acid sequence set forth in SEQ ID NO:23. For example, an antigen binding domain that can bind to a CD3 polypeptide can include a light chain including the amino acid sequence set forth in SEQ ID NO:42.

In some cases, an antigen binding domain that can bind to a CD3 polypeptide can include a light chain having a V_{L} CDR1 including the amino acid sequence set forth in SEQ ID NO:44, a V_{L} CDR2 including the amino acid sequence set forth in SEQ ID NO:48, and a V_{L} CDR3 including the amino acid sequence set forth in SEQ ID NO:51. For example, an antigen binding domain that can bind to a CD3 polypeptide can include a light chain including the amino acid sequence set forth in SEQ ID NO:61.

In some cases, an antigen binding domain that can bind to a CD3 polypeptide can include a light chain having a V_{L} CDR1 including the amino acid sequence set forth in SEQ ID NO:45, a V_{L} CDR2 including the amino acid sequence set forth in SEQ ID NO:49, and a V_{L} CDR3 including the amino acid sequence set forth in SEQ ID NO:52. For example, an antigen binding domain that can bind to a CD3 polypeptide can include a light chain including the amino acid sequence set forth in SEQ ID NO:63.

In some cases, an antigen binding domain that can bind to a CD3 polypeptide can include a light chain having a V_{L} CDR1 including the amino acid sequence set forth in SEQ ID NO:46, a V_{L} CDR2 including the amino acid sequence set forth in SEQ ID NO:50, and a V_{L} CDR3 including the amino acid sequence set forth in SEQ ID NO:53. For example, an antigen binding domain that can bind to a CD3 polypeptide can include a light chain including the amino acid sequence set forth in SEQ ID NO:65.

In some cases, an antigen binding domain that can bind to a CD3 polypeptide can include a light chain having a V_{L} CDR1 including the amino acid sequence set forth in SEQ ID NO:47, a V_{L} CDR2 including the amino acid sequence set forth in SEQ ID NO:48, and a V_{L} CDR3 including the amino acid sequence set forth in SEQ ID NO:51. For example, an antigen binding domain that can bind to a CD3 polypeptide can include a light chain including the amino acid sequence set forth in SEQ ID NO:67.

In some cases, an antigen binding domain that can bind to a CD3 polypeptide can include a heavy chain having a V_{H} CDR1 including the amino acid sequence set forth in SEQ ID NO:20, a V_{H} CDR2 including the amino acid sequence set forth in SEQ ID NO:21, and a V_{H} CDR3 including the amino acid sequence set forth in SEQ ID NO:22. For example, an antigen binding domain that can bind to a CD3 polypeptide can include a heavy chain including the amino acid sequence set forth in SEQ ID NO:24. For example, an antigen binding domain that can bind to a CD3 polypeptide can include a heavy chain including the amino acid sequence set forth in SEQ ID NO:43.

In some cases, an antigen binding domain that can bind to a CD3 polypeptide can include a heavy chain having a V_{H} CDR1 including the amino acid sequence set forth in SEQ ID NO:54, a V_{H} CDR2 including the amino acid sequence set forth in SEQ ID NO:56, and a V_{H} CDR3 including the amino acid sequence set forth in SEQ ID NO:59. For example, an antigen binding domain that can bind to a CD3 polypeptide can include a heavy chain including the amino acid sequence set forth in SEQ ID NO:62.

In some cases, an antigen binding domain that can bind to a CD3 polypeptide can include a heavy chain having a V_{H} CDR1 including the amino acid sequence set forth in SEQ ID NO:54, a V_{H} CDR2 including the amino acid sequence set forth in SEQ ID NO:56, and a V_{H} CDR3 including the amino acid sequence set forth in SEQ ID NO:59. For example, an antigen binding domain that can bind to a CD3 polypeptide can include a heavy chain including the amino acid sequence set forth in SEQ ID NO:64.

In some cases, an antigen binding domain that can bind to a CD3 polypeptide can include a heavy chain having a V_{H} CDR1 including the amino acid sequence set forth in SEQ ID NO:55, a V_{H} CDR2 including the amino acid sequence set forth in SEQ ID NO:57, and a V_{H} CDR3 including the amino acid sequence set forth in SEQ ID NO:60. For example, an antigen binding domain that can bind to a CD3 polypeptide can include a heavy chain including the amino acid sequence set forth in SEQ ID NO:66.

In some cases, an antigen binding domain that can bind to a CD3 polypeptide can include a heavy chain having a V_{H} CDR1 including the amino acid sequence set forth in SEQ ID NO:54, a V_{H} CDR2 including the amino acid sequence set forth in SEQ ID NO:58, and a V_{H} CDR3 including the amino acid sequence set forth in SEQ ID NO:59. For example, an antigen binding domain that can bind to a CD3 polypeptide can include a heavy chain including the amino acid sequence set forth in SEQ ID NO:68.

In some cases, an antigen binding domain that can bind to a CD3 polypeptide can include a light chain having a V_{L} CDR1 including the amino acid sequence set forth in SEQ ID NO:17, a V_{L} CDR2 including the amino acid sequence set forth in SEQ ID NO: 18, and a V_{L} CDR3 including the amino acid sequence set forth in SEQ ID NO: 19, and can include a heavy chain having a V_{H} CDR1 including the amino acid sequence set forth in SEQ ID NO:20, a V_{H} CDR2 including the amino acid sequence set forth in SEQ ID NO:21, and a V_{H} CDR3 including the amino acid sequence set forth in SEQ ID NO:22. For example, an antigen binding domain that can bind to a CD3 polypeptide can include a light chain including the amino acid sequence set forth in SEQ ID NO:23 and can include a heavy chain including the amino acid sequence set forth in SEQ ID NO:24. For example, an antigen binding domain that can bind to a CD3 polypeptide can include a light chain including the amino acid sequence set forth in SEQ ID NO:42 and can include a heavy chain including the amino acid sequence set forth in SEQ ID NO:43.

In some cases, an antigen binding domain that can bind to a CD3 polypeptide can include a light chain having a V_{L} CDR1 including the amino acid sequence set forth in SEQ ID NO:44, a V_{L} CDR2 including the amino acid sequence set forth in SEQ ID NO:48, and a V_{L} CDR3 including the amino acid sequence set forth in SEQ ID NO:51, and can include a heavy chain having a V_{H} CDR1 including the amino acid sequence set forth in SEQ ID NO:54, a V_{H} CDR2 including the amino acid sequence set forth in SEQ ID NO:56, and a V_{H} CDR3 including the amino acid sequence set forth in SEQ ID NO:59. For example, an antigen binding domain that can bind to a CD3 polypeptide can include a light chain including the amino acid sequence set forth in SEQ ID NO:61 and can include a heavy chain including the amino acid sequence set forth in SEQ ID NO:62.

In some cases, an antigen binding domain that can bind to a CD3 polypeptide can include a light chain having a V_{L} CDR1 including the amino acid sequence set forth in SEQ ID NO:45, a V_{L} CDR2 including the amino acid sequence set forth in SEQ ID NO:49, and a V_{L} CDR3 including the amino acid sequence set forth in SEQ ID NO:52, and can include a heavy chain having a V_{H} CDR1 including the amino acid sequence set forth in SEQ ID NO:54, a V_{H} CDR2 including the amino acid sequence set forth in SEQ ID NO:56, and a V_{H} CDR3 including the amino acid sequence set forth in SEQ ID NO:59. For example, an antigen binding domain that can bind to a CD3 polypeptide can include a light chain including the amino acid sequence set forth in SEQ ID NO:63 and can include a heavy chain including the amino acid sequence set forth in SEQ ID NO:64.

In some cases, an antigen binding domain that can bind to a CD3 polypeptide can include a light chain having a V_{L} CDR1 including the amino acid sequence set forth in SEQ ID NO:46, a V_{L} CDR2 including the amino acid sequence set forth in SEQ ID NO:50, and a V_{L} CDR3 including the amino acid sequence set forth in SEQ ID NO:53, and can include a heavy chain having a V_{H} CDR1 including the amino acid sequence set forth in SEQ ID NO:55, a V_{H} CDR2 including the amino acid sequence set forth in SEQ ID NO:57, and a V_{H} CDR3 including the amino acid sequence set forth in SEQ ID NO:60. For example, an antigen binding domain that can bind to a CD3 polypeptide can include a light chain including the amino acid sequence set forth in SEQ ID NO:65 and can include a heavy chain including the amino acid sequence set forth in SEQ ID NO:66.

In some cases, an antigen binding domain that can bind to a CD3 polypeptide can include a light chain having a V_{L} CDR1 including the amino acid sequence set forth in SEQ ID NO:47, a V_{L} CDR2 including the amino acid sequence set forth in SEQ ID NO:48, and a V_{L} CDR3 including the amino acid sequence set forth in SEQ ID NO:51, and can include a heavy chain having a V_{H} CDR1 including the amino acid sequence set forth in SEQ ID NO:54, a V_{H} CDR2 including the amino acid sequence set forth in SEQ ID NO:58, and a V_{H} CDR3 including the amino acid sequence set forth in SEQ ID NO:59. For example, an antigen binding domain that can bind to a CD3 polypeptide can include a light chain including the amino acid sequence set forth in SEQ ID NO:67 and can include a heavy chain including the amino acid sequence set forth in SEQ ID NO:68.

In some cases, a second antigen binding domain in a bispecific molecule provided herein (e.g., a bispecific molecule including a first antigen binding domain that can bind a TRBV polypeptide and a second antigen binding domain that can bind a T cell co-receptor polypeptide) can be as described elsewhere (see, e.g., Zhu et al., Journal of Immunology, 155:1903-1910 (1995); and Junttila et al., Cancer Research, 74:5561-5571 (2014)).

In some cases, a first antigen binding domain and a second antigen binding domain in a bispecific molecule provided herein (e.g., a bispecific molecule including a first antigen binding domain that can bind a TRBV polypeptide and a second antigen binding domain that can bind a T cell co-receptor polypeptide) can be connected via a linker (e.g., a polypeptide linker). A linker can include any appropriate number of amino acids. For example, a linker can include from about 5 amino acids to about 20 amino acids (e.g., from about 5 amino acids to about 18 amino acids, from about 5 amino acids to about 15 amino acids, from about 5 amino acids to about 12 amino acids, from about 5 amino acids to about 10 amino acids, from about 5 amino acids to about 8 amino acids, from about 7 amino acids to about 20 amino acids, from about 10 amino acids to about 20 amino acids, from about 12 amino acids to about 20 amino acids, from about 16 amino acids to about 20 amino acids, from about 8 amino acids to about 16 amino acids, from about 10 amino acids to about 12 amino acids, from about 8 amino acids to about 12 amino acids, from about 10 amino acids to about 15 amino acids, or from about 12 amino acids to about 16 amino acids). In some cases, a linker can alter the flexibility of the bispecific molecule. In some cases, a linker can alter the solubility of the bispecific molecule. A linker can include any appropriate amino acids. In some cases, a linker can be a glycine-rich linker. In some cases, a linker can be serine and/or threonine-rich linker. A linker can connect the first antigen binding domain and the second antigen binding domain in a bispecific molecule provided herein in any order. For example, a linker can connect the N-terminus of a first antigen binding domain in a bispecific molecule provided herein with the C-terminus of the second antigen binding domain in a bispecific molecule, or vice versa. Examples of linkers that can be used to connect a first antigen binding domain and a second antigen binding domain in a bispecific molecule provided herein include, without limitation, a GGGGS linker (SEQ ID NO:25), a (GGGGS)₃ linker (SEQ ID NO:26), and GGSGGSGGSGGSGGVD (SEQ ID NO:69).

In some cases, one or more bispecific molecules provided herein (e.g., bispecific molecules including a first antigen binding domain that can bind a TRBV polypeptide and a second antigen binding domain that can bind a T cell co-receptor polypeptide) can be formulated into a composition (e.g., a pharmaceutical composition) for administration to a mammal (e.g., a human). For example, one or more bispecific molecules provided herein can be formulated into a pharmaceutically acceptable composition for administration to a mammal (e.g., a human) having a T cell cancer. In some cases, one or more bispecific molecules provided herein can be formulated together with one or more pharmaceutically acceptable carriers (additives), excipients, and/or diluents. Examples of pharmaceutically acceptable carriers, excipients, and diluents that can be used in a composition described herein include, without limitation, sucrose, lactose, starch (e.g., starch glycolate), cellulose, cellulose derivatives (e.g., modified celluloses such as microcrystalline cellulose and cellulose ethers like hydroxypropyl cellulose (HPC) and cellulose ether hydroxypropyl methylcellulose (HPMC)), xylitol, sorbitol, mannitol, gelatin, polymers (e.g., polyvinylpyrrolidone (PVP), polyethylene glycol (PEG), crosslinked polyvinylpyrrolidone (crospovidone), carboxymethyl cellulose, polyethylene-polyoxypropylene-block polymers, and crosslinked sodium carboxymethyl cellulose (croscarmellose sodium)), titanium oxide, azo dyes, silica gel, fumed silica, talc, magnesium carbonate, vegetable stearin, magnesium stearate, aluminum stearate, stearic acid, antioxidants (e.g., vitamin A, vitamin E, vitamin C, retinyl palmitate, and selenium), citric acid, sodium citrate, parabens (e.g., methyl paraben and propyl paraben), petrolatum, dimethyl sulfoxide, mineral oil, serum proteins (e.g., human serum albumin), glycine, sorbic acid, potassium sorbate, water, salts or electrolytes (e.g., saline, protamine sulfate, disodium hydrogen phosphate, potassium hydrogen phosphate, sodium chloride, and zinc salts), colloidal silica, magnesium trisilicate, polyacrylates, waxes, wool fat, and lecithin.

A composition (e.g., a pharmaceutical composition) containing one or more bispecific molecules provided herein (e.g., bispecific molecules including a first antigen binding domain that can bind a TRBV polypeptide and a second antigen binding domain that can bind a T cell co-receptor polypeptide) can be formulated into any appropriate dosage form. Examples of dosage forms include solid or liquid forms including, without limitation, pills, capsules, tablets, gels, liquids, suspensions, solutions (e.g., sterile solutions), sustained-release formulations, and delayed-release formulations.

A composition (e.g., a pharmaceutical composition) containing one or more bispecific molecules provided herein (e.g., bispecific molecules including a first antigen binding domain that can bind a TRBV polypeptide and a second antigen binding domain that can bind a T cell co-receptor polypeptide) can be designed for oral or parenteral (e.g., topical, subcutaneous, intravenous, intraperitoneal, intrathecal, and intraventricular) administration. When being administered orally, a composition can be in the form of a pill, tablet, or capsule. Compositions suitable for parenteral administration include aqueous and non-aqueous sterile injection solutions that can contain anti-oxidants, buffers, bacteriostats, and solutes which render the formulation isotonic with the blood of the intended recipient; and aqueous and non-aqueous sterile suspensions which may include suspending agents and thickening agents. The formulations can be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules, and tablets.

A composition (e.g., a pharmaceutical composition) containing one or more bispecific molecules provided herein (e.g., bispecific molecules including a first antigen binding domain that can bind a TRBV polypeptide and a second antigen binding domain that can bind a T cell co-receptor polypeptide) can be administered locally or systemically. For example, a composition containing one or more bispecific molecules provided herein can be administered systemically by an intravenous injection to a mammal (e.g., a human). For example, a composition containing one or more bispecific molecules provided herein can be administered systemically by a subcutaneous injection to a mammal (e.g., a human).

An effective amount (e.g., effective dose) of one or more bispecific molecules provided herein (e.g., bispecific molecules including a first antigen binding domain that can bind a TRBV polypeptide and a second antigen binding domain that can bind a T cell co-receptor polypeptide) can vary depending on the severity of the T cell cancer, the route of administration, the age and general health condition of the subject, excipient usage, the possibility of co-usage with other therapeutic treatments such as use of other agents, and/or the judgment of the treating physician.

An effective amount of a composition (e.g., a pharmaceutical composition) containing one or more bispecific molecules provided herein (e.g., bispecific molecules including a first antigen binding domain that can bind a TRBV polypeptide and a second antigen binding domain that can bind a T cell co-receptor polypeptide) can be any amount that can treat a mammal (e.g., a human) having a T cell cancer without producing significant toxicity to the mammal. An effective amount of one or more bispecific molecules provided herein can be any appropriate amount. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition (e.g., a T cell cancer) may require an increase or decrease in the actual effective amount administered.

The frequency of administration of a composition (e.g., a pharmaceutical composition) containing one or more bispecific molecules provided herein (e.g., bispecific molecules including a first antigen binding domain that can bind a TRBV polypeptide and a second antigen binding domain that can bind a T cell co-receptor polypeptide) can be any frequency that can treat a mammal (e.g., a human) having a T cell cancer without producing significant toxicity to the mammal. For example, the frequency of administration can be once a day, once a week, once every 2 weeks, or once every 4 weeks. In some cases, an administration can include a continuous infusion of a composition containing one or more bispecific molecules provided herein. The frequency of administration can remain constant or can be variable during the duration of treatment. A course of treatment with a composition containing one or more bispecific molecules provided herein can include rest periods. As with the effective amount, various factors can influence the actual frequency of administration used for a particular application. For example, the effective amount, duration of treatment, use of multiple treatment agents, route of administration, and severity of the condition (e.g., a T cell cancer) may require an increase or decrease in administration frequency.

An effective duration for administering a composition (e.g., a pharmaceutical composition) containing one or more bispecific molecules provided herein (e.g., bispecific molecules including a first antigen binding domain that can bind a TRBV polypeptide and a second antigen binding domain that can bind a T cell co-receptor polypeptide) can be any duration that treat a mammal (e.g., a human) having a T cell cancer without producing significant toxicity to the mammal. For example, the effective duration can vary from several days to several weeks, months, or years. In some cases, the effective duration for the treatment of a mammal can range in duration from about one month to about 10 years. Multiple factors can influence the actual effective duration used for a particular treatment. For example, an effective duration can vary with the frequency of administration, effective amount, use of multiple treatment agents, route of administration, and severity of the condition (e.g., a T cell cancer) being treated.

In some cases, one or more bispecific molecules provided herein (e.g., bispecific molecules including a first antigen binding domain that can bind a TRBV polypeptide and a second antigen binding domain that can bind a T cell co-receptor polypeptide) can be used as the sole active agent to treat a mammal (e.g., a human) having a T cell cancer.

In some cases, the methods and materials described herein can include one or more (e.g., one, two, three, four, five or more) additional therapeutic agents used to treat a mammal (e.g., a human) having a T cell cancer. For example, a mammal in need thereof (e.g., a mammal having a T cell cancer) can be administered one or more bispecific molecules provided herein (e.g., bispecific molecules including a first antigen binding domain that can bind a TRBV polypeptide and a second antigen binding domain that can bind a T cell co-receptor polypeptide) in combination with one or more anti-cancer agents. In some cases, an anti-cancer agent can be an alkylating agent. In some cases, an anti-cancer agent can be a platinum compound. In some cases, an anti-cancer agent can be a taxane. In some cases, an anti-cancer agent can be a luteinizing-hormone-releasing hormone (LHRH) agonist. In some cases, an anti-cancer agent can be an anti-estrogen. In some cases, an anti-cancer agent can be an aromatase inhibitor. In some cases, an anti-cancer agent can be an angiogenesis inhibitor. In some cases, an anti-cancer agent can be a poly(ADP)-ribose polymerase (PARP) inhibitor. In some cases, an anti-cancer agent can be a topoisomerase inhibitor. In some cases, an anti-cancer agent can be a corticosteroid. In some cases, an anti-cancer agent can be an antibody. In some cases, an anti-cancer agent can be an antibody drug conjugate. Examples of anti-cancer agents include, without limitation, busulfan, cisplatin, carboplatin, paclitaxel, docetaxel, nab-paclitaxel, altretamine, capecitabine, cyclophosphamide, etoposide (vp-16), gemcitabine, ifosfamide, irinotecan (cpt-11), melphalan, pemetrexed, topotecan, vinorelbine, goserelin, leuprolide, tamoxifen, letrozole, anastrozole, exemestane, bevacizumab, olaparib, rucaparib, niraparib, cyclophosphamide, doxorubicin (e.g., liposomal doxorubicin), prednisone, prednisolone, dexamethasone, mogamulizumab, brentuximab, and any combinations thereof. In some cases, the one or more additional therapeutic agents can be administered together with one or more bispecific molecules provided herein (e.g., in a single composition). In some cases, the one or more additional therapeutic agents can be administered independent of the one or more bispecific molecules provided herein. When the one or more additional therapeutic agents are administered independent of the one or more bispecific molecules provided herein, the one or more bispecific molecules provided herein can be administered first, and the one or more additional therapeutic agents administered second, or vice versa.

In some cases, the methods and materials described herein can include one or more (e.g., one, two, three, four, five or more) additional treatments (e.g., therapeutic interventions) that are effective to treat T cell cancers. For example, a mammal in need thereof (e.g., a mammal having a T cell cancer) can be administered one or more bispecific molecules provided herein (e.g., bispecific molecules including a first antigen binding domain that can bind a TRBV polypeptide and a second antigen binding domain that can bind a T cell co-receptor polypeptide) in combination with one or more therapeutic interventions. Examples of therapeutic interventions that can be used as described herein to treat a T cell cancer include, without limitation, cancer surgeries, radiation therapies, chemotherapies, and any combinations thereof. In some cases, the one or more additional treatments that are effective to treat T cell cancers can be performed at the same time as the administration of the one or more bispecific molecules provided herein. In some cases, the one or more additional treatments that are effective to treat T cell cancers can be performed before and/or after the administration of the one or more bispecific molecules provided herein.

In some cases, one or more bispecific molecules provided herein (e.g., bispecific molecules including a first antigen binding domain that can bind a TRBV polypeptide and a second antigen binding domain that can bind a T cell co-receptor polypeptide) can be used to treat a mammal having a disease or disorder other than cancer. For example, a mammal having a disease, disorder, or condition other than a T cell cancer that is associated with a clonal T cell expansion can be administered one or more bispecific molecules provided herein. In some cases, a disease, disorder, or condition other than a T cell cancer that is associated with a clonal T cell expansion can be an autoimmune disease. In some cases, a disease, disorder, or condition other than a T cell cancer that is associated with a clonal T cell expansion can be associated with transplant rejection. Examples of diseases and disorders associated with a clonal T cell expansion that can be targeted using one or more bispecific molecules provided herein include, without limitation, graft versus host disease (GVHD), celiac disease, and multiple sclerosis.

In some cases, the materials and methods described herein can be used to treat a mammal (e.g., a human) having celiac disease. For example, one or more bispecific molecules provided herein (e.g., bispecific molecules including a first antigen binding domain that can bind a TRBV polypeptide and a second antigen binding domain that can bind a T cell co-receptor polypeptide) can be administered to a mammal (e.g., a human) having celiac disease to treat the mammal. In some cases, a bispecific molecule including a first antigen binding domain that can bind a TRBV polypeptide associated with celiac disease and a second antigen binding domain that can bind a T cell co-receptor polypeptide (e.g., a CD3 polypeptide) can be administered to a mammal (e.g., a human) having celiac disease to treat the mammal. In some cases where a bispecific molecule provided herein is used to treat a mammal having celiac disease, the first antigen binding domain of the bispecific molecule can bind a TRBV polypeptide selected from the group consisting of TRBV4, TRBV6, TRBV7, TRBV9, TRBV20 and TRBV29, and the second antigen binding domain can bind a T cell co-receptor polypeptide (e.g., a CD3 polypeptide). In some cases where a bispecific molecule provided herein is used to treat a mammal having celiac disease, the first antigen binding domain of the bispecific molecule can bind a TRBV polypeptide selected from the group consisting of TRBV6-1, TRBV7-2, TRBV9-1, TRBV20-1 and TRBV29-1, and the second antigen binding domain can bind a T cell co-receptor polypeptide (e.g., a CD3 polypeptide).

In some cases, a first antigen binding domain described herein (e.g., a first antigen binding domain that can bind a TRBV polypeptide) can be included in chimeric antigen receptor (CAR) that can be presented on a T cell (a CAR T cell). For example, a CAR T cell that includes an antigen binding domain that can bind a TRBV polypeptide can be used to treat a mammal having a T cell cancer. In some cases, a mammal having a T cell cancer can be administered CAR T cells that include an antigen binding domain that can bind a TRBV polypeptide provided herein to treat the mammal. A CAR T cell that includes an antigen binding domain that can bind a TRBV polypeptide can be used in any type of CAR T cell therapy. CAR T cell therapies can include those as described elsewhere (see, e.g., Ali et al., Blood, 128(13):1688-700 (2016); Sadelain et al., Cancer Discov., 3(4):388-98 (2013); Porter et al., N. Engl. J. Med., 365(8):725-33 (2011); and Maciocia et al., Nat. Med., 23(12):1416-1423 (2017)).

In some cases, a first antigen binding domain described herein (e.g., a first antigen binding domain that can bind a TRBV polypeptide) can be included in an antibody drug conjugate (ADC). For example, an ADC that includes an antigen binding domain that can bind a TRBV polypeptide can be used to treat a mammal having a T cell cancer. In some cases, a mammal having a T cell cancer can be administered an ADC that includes an antigen binding domain that can bind a TRBV polypeptide provided herein to treat the mammal. An ADC that includes an antigen binding domain that can bind a TRBV polypeptide can include any type of drug. Drugs that can be used in an ADC can include those as described elsewhere (see, e.g., Younes et al., Lancet Oncol., 14(13):1348-56 (2013); Hamblett et al., Clin. Cancer. Res., 10(20):7063-70 (2004); and Lewis Phillips et al., Cancer Res., 68(22):9280-90 (2008)).

The invention will be further described in the following examples, which do not limit the scope of the invention described in the claims.

### EXAMPLES

### Example 1: TCR Beta Chain-Directed Bispecifrc Antibodies for the Treatment of T Cell Cancers

This Examples describe the generation and evaluation of a TRBC targeting BsAbs and two different TRBV targeting BsAbs for the treatment of T cell cancers. The TRBC-targeting BsAb can eradicate both the T cell cancers and the vast majority of healthy human (normal) T cells due to bidirectional T cell killing. The TRBV-targeting BsAbs can deplete cancerous T cells in vitro and in vivo while preserving the majority of normal T cells.

### Results

### BsAb Targeting of Normal T Cells

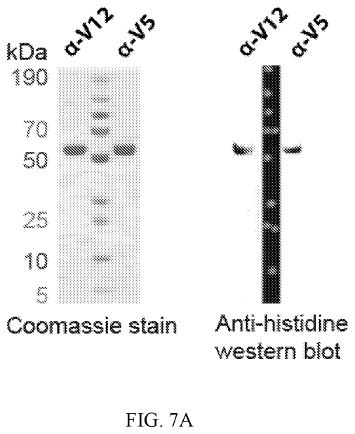

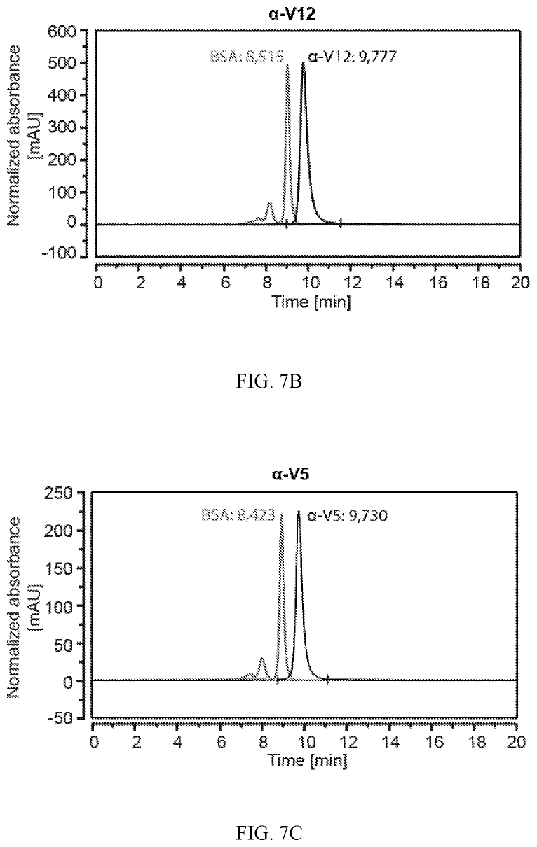

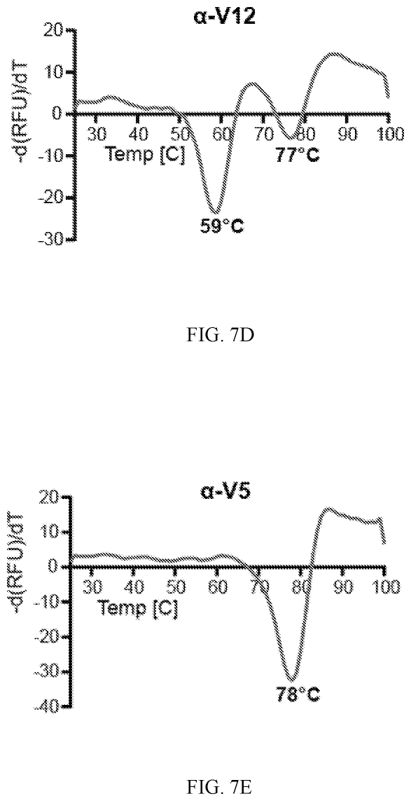

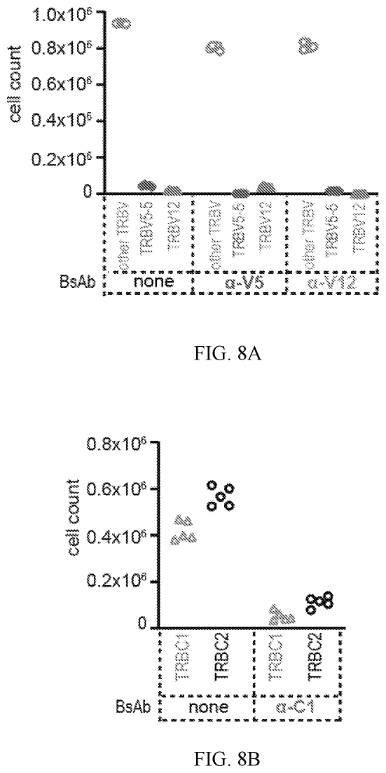

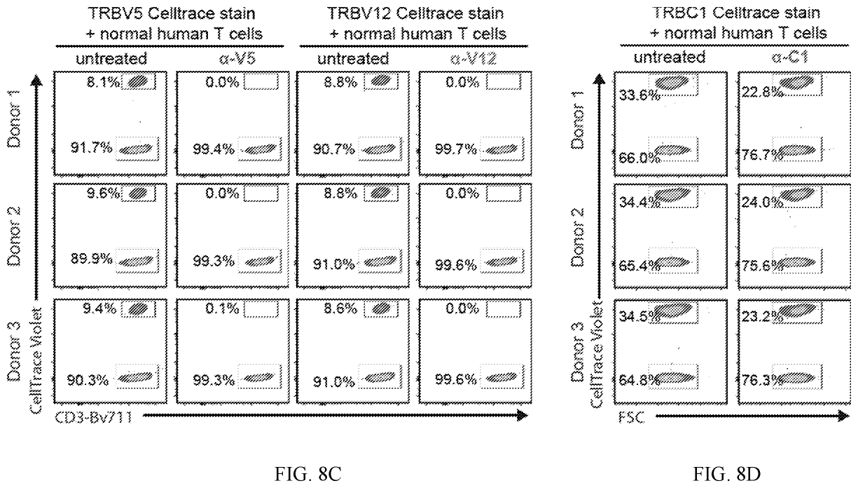

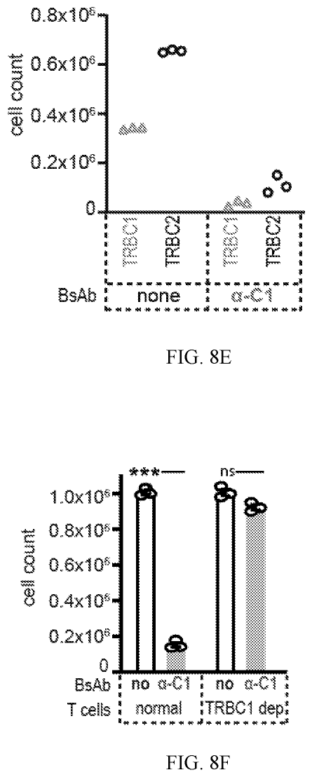

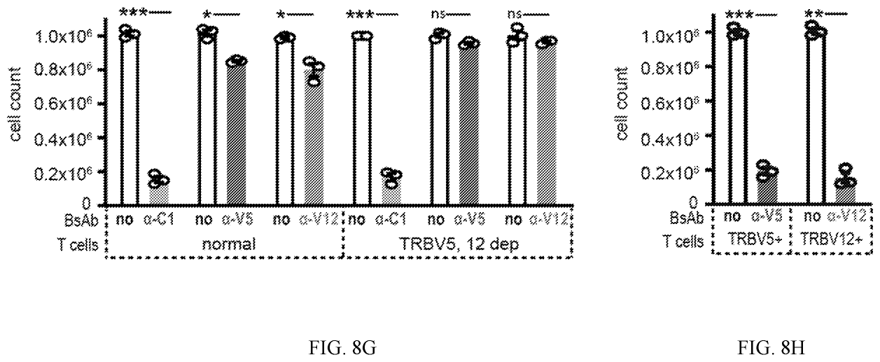

Anti-TRBV5-5 and anti-TRBV12 scFv sequences were used to generate anti-TRBV5-5 and anti-TRBV12 BsAbs (henceforth denoted “α-V5” and “α-V12”) for selective targeting of TRBV5-5⁺ or TRBV12⁺ T cells, respectively (FIG. 1B, FIG. 7A, and Table 1). Similarly, anti-TRBC1 BsAbs (henceforth denoted “α-C1”) were generated for selective targeting of TRBC1⁺ T cells (FIG. 1B and Table 1). Analytic chromatography showed monomeric BsAbs with >99% purity (FIG. 7B, C). Thermal stability of α-V12 and α-V5 were evaluated using differential scanning fluorimetry. α-V5 presented a single melting temperature (Tₘ) at 78° C., and α-V12 showed two Tₘ at 59° C. and 77° C. (FIG. 7D, E). These data suggest that for α-V5, both the anti-TRBV5-5 scFv and the anti-CD3 scFv unfold at 78° C., while for α-V12, the anti-TRBV12 scFv unfolds at 59° C. and the anti-CD3 scFv unfolds at 77° C. It was found that between 1.5 to 2% and 3.5% to 5% of normal human T cells express TRBV5-5 and TRBV12 (FIG. 1C, D), respectively. Approximately 35-45% human T cells express TRBC1 and the rest express TRBC2 (FIG. 1C, E). In vitro exposure of T cells from healthy individuals to α-V5 and α-V12 treatment resulted in complete loss of the TRBV5-5⁺ and TRBV12⁺ cells, respectively (FIG. 1C, D and FIG. 8A). Similarly, exposure of T cells from healthy individuals to α-C1 resulted in a substantial loss of TRBC1⁺ T cells (FIG. 1C, E and FIG. 8B). However, there was a major difference in the loss of the non-targeted T cells mediated by the TRBV- and TRBC-specific BsAbs. The α-V5 BsAb depleted 14.1% (mean) of the T cells not expressing TRBV5-5, and α-V12 eradicated 13.3% (mean) of the T cells not expressing TRBV12 (FIG. 1C). In contrast, α-C1 eradicated 80.0% (mean) of the T cells not expressing TRBC1 (FIG. 1C). Consequently, α-C1 resulted in depletion of the majority of the total T cells, while α-V5 or α-V12 preserved the majority of T cells (FIG. 1C). To confirm that BsAb mediated TCR internalization or TCR epitope blocking are not interfering with subsequent antibody based analysis of different T cell subtypes, CellTrace Violet stained target cells (TRBV5⁺, TRBV12⁺ or TRBC1⁺ T cells) were utilized. α-V5 and α-V12 exposure led to depletion of CellTrace Violet stained TRBV5 and TRBV12⁺ cells (FIG. 8C), and α-C1 caused substantial loss of both TRBC1⁺ and TRBC2⁺ cells (FIG. 8D, E).

TABLE 1
Affinities and sequences of α-TRBV5-5, α-TRBV12, α-TRBC1, α-CD19 and α-CD3 scFvs.

| scFv | Affinity | V_{L} | SEQ ID | V_{H} | SEQ ID |
|---|---|---|---|---|---|
| α-TRBV5-5 | 25.2 nM | DIQMTQTTSSLSASLGDRVTITCSASQGIS | 7 | QVQLKESGPGLVAPSQSLSITCTVSGFSLTA | 8 |
| (clone TM23) | | NYLNWYQQKPDGTVKLLIYYTSSLHSGV | | YGVNWVRQPPGKGLEWLGMIWGDGNTD | |
| | | PSRFSGSGSGTDYSLTISNLEPEDIATYYC | | YNSALKSRLSISKDNSKSQVFLKMNSLQTD | |
| | | QQYSKLPRTFGGGTKVEIK | | DTARYYCARDRVTATLYAMDYWGQGTSV | |
| | | | | TVSS | |
| α-TRBV12 | 2.6 nM | ENVLTQSPAIMSASLGEKVTMSCRASSSV | 15 | DVQLVESGGGLVQPGGSRKLSCAASGFTFS | 16 |
| (clone 16G8) | | NYIYWYQQKSDASPKLWIYYTSNLAPGV | | NFGMHWVRQAPGKGLEWVAYISSGSSTIY | |
| | | PTRFSGSGSGNSYSLTISSMEGEDAATYY | | YADTLKGRFTISRDNPKNTLFLQMTSLRSE | |
| | | CQQFTSSPFTFGQGTKLEIK | | DTAMYYCARRGEGAMDYWGQGTSVTVSS | |
| α-TRBC1 | 0.4 nM | DVVMTQSPLSLPVSLGDQASISCRSSQRL | 27 | EVRLQQSGPDLIKPGASVKMSCKASGYTFT | 28 |
| (clone Jovi-1) | | VHSNGNTYLHWYLQKPGQSPKLLIYRVS | | GYVMHWVKQRPGQGLEWIGFINPYNDDIQ | |
| | | NRFPGVPDRFSGSGSGTDFTLKISRVEAED | | SNERFRGKATLTSDKSSTTAYMELSSLTSE | |
| | | LGIYFCSQSTHVPYTFGGGTKLEIKR | | DSAVYYCARGAGYNFDGAYRFFDFWGQG | |
| | | | | TTLTVSS | |
| α-CD19 (clone | 0.3 nM | DIQLTQSPASLAVSLGQRATISCKASQSVD | 29 | QVQLQQSGAELVRPGSSVKISCKASGYAFS | 30 |
| HD37) | | YDGDSYLNWYQQIPGQPPKLLIYDASNL | | SYWMNWVKQRPGQGLEWIGQIWPGDGDT | |
| | | VSGIPPRFSGSGSGTDFTLNIHPVEKVDAA | | NYNGKFKGKATLTADESSSTAYMQLSSLA | |
| | | TYHCQQSTEDPWTFGGGTKLEIK | | SEDSAVYFCARRETTTVGRYYYAMDYWG | |
| | | | | QGTTVTVSS | |
| α-CD3 (clone | 4.7 nM | DIQMTQSPSSLSASVGDRVTITCRASQDIR | 23 | EVQLVESGGGLVQPGGSLRLSCAASGYSFT | 24 |
| UCHT1.v9) | | NYLNWYQQKPGKAPKLLIYYTSRLESGV | | GYTMNWVRQAPGKGLEWVALINPYKGVS | |
| | | PSRFSGSGSGTDYTLTISSLQPEDFATYYC | | TYNQKFKDRFTISVDKSKNTAYLQMNSLR | |
| | | QQGNTLPWTFGQGTKVEIK | | AEDTAVYYCARSGYYGDSDWYFDVWGQ | |
| | | | | GTLVTVSS | |

Basis for BsAb Targeting of T Cells that do not Express the Relevant TRBV or TRBC

To examine whether BsAbs result in killing of T cells not expressing the relevant TRBV or TRBC chain, TRBC1V cells were depleted from human T cells, then the depleted T cells were exposed to α-C1. After depletion of T cells expressing TRBC1, exposure to α-C1 did not result in statistically significant killing of the remaining T cells (FIG. 8F). Similarly, after depletion of T cells expressing either TRBV5 or TRBV12, exposure of α-V5 or α-V12 did not result in statistically significant killing of the remaining T cells (FIG. 8G). Additionally, a pure population of TRBV5⁺ or TRBV12⁺ T cells experienced almost complete cell loss after exposure to α-V5 and α-V12 respectively (FIG. 8H). These results suggested that the effects of all three BsAbs were dependent on the presence of the relevant TRBV or TRBC chains in the treated T cells.

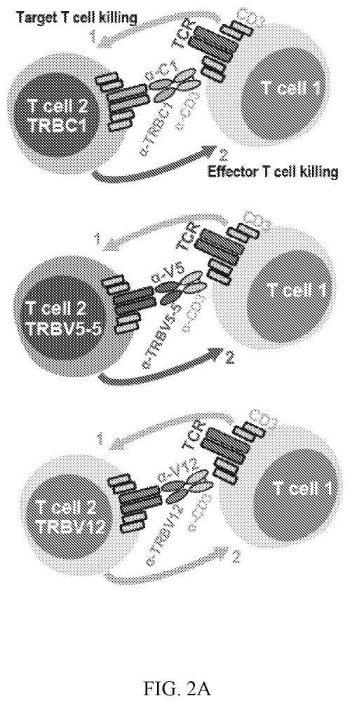

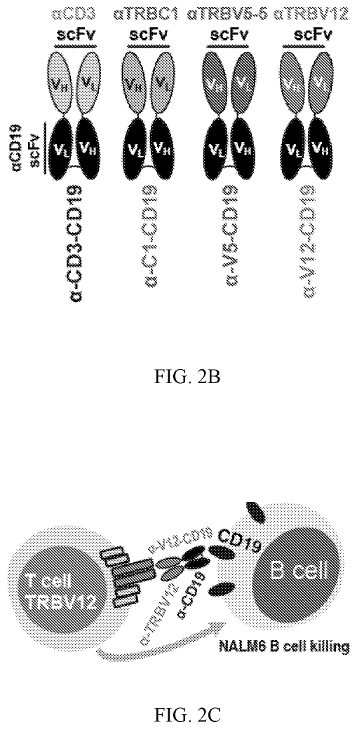

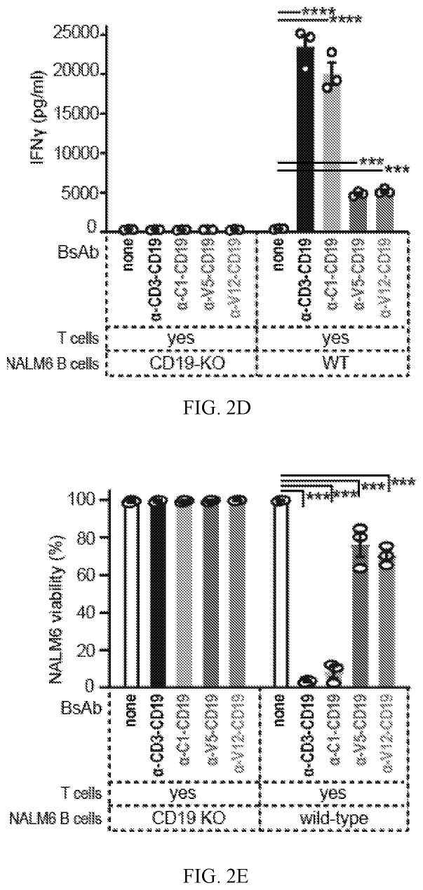

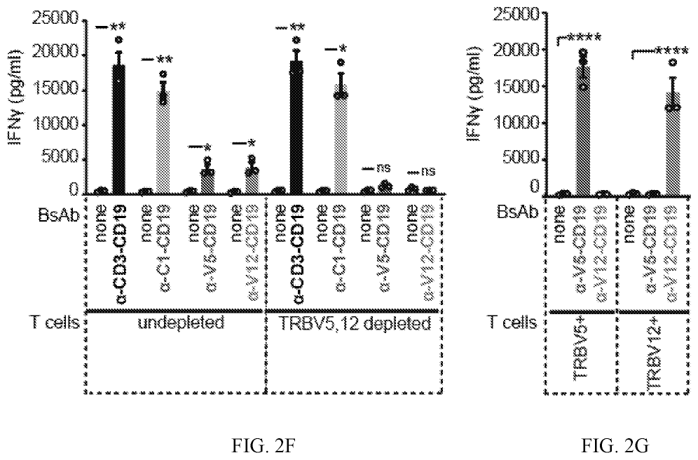

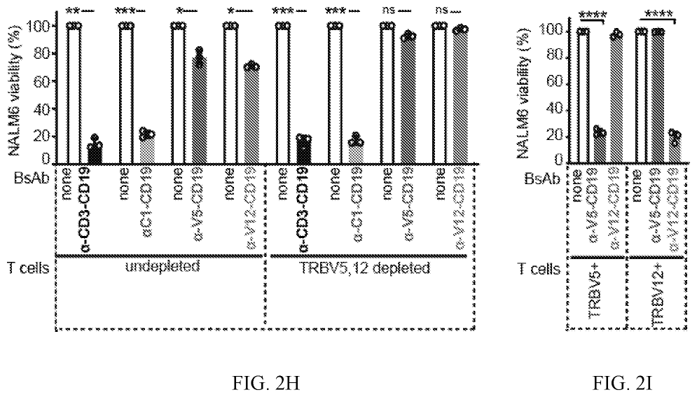

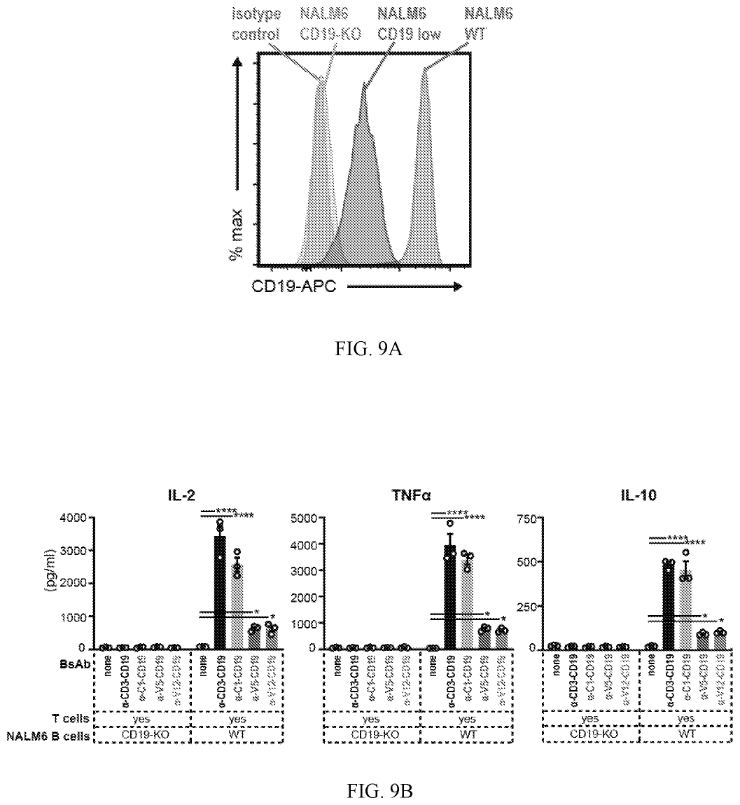

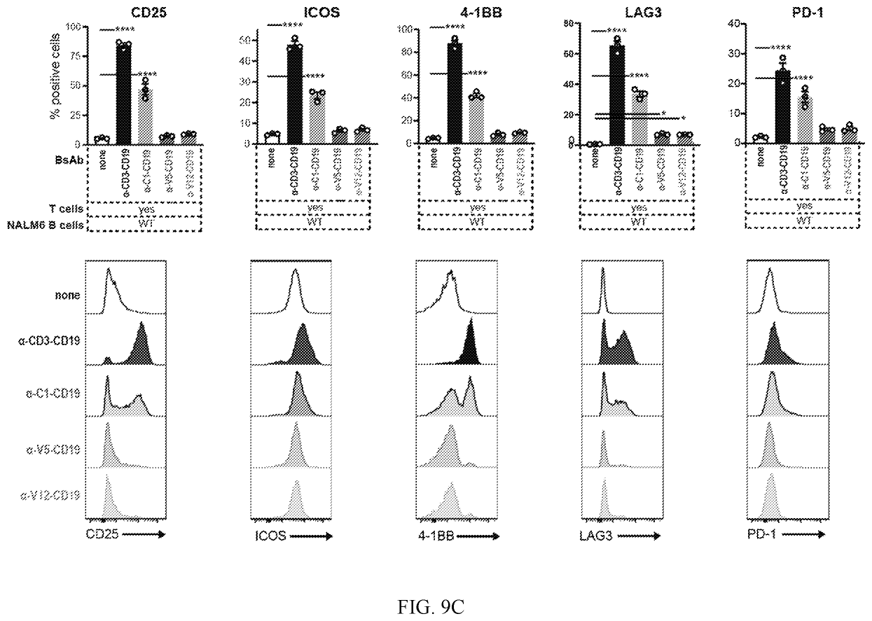

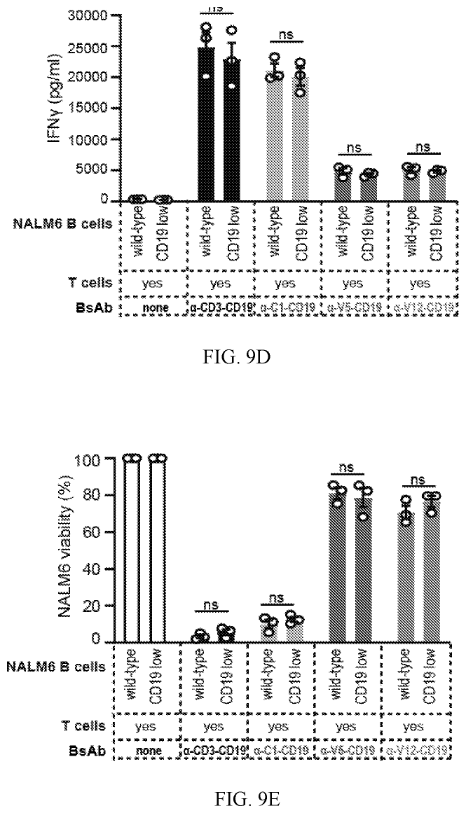

Exemplary BsAb molecules (FIG. 1B) can be composed of one scFv arm interacting with a TRBC or a TRBV region (expressed only by target T cell subset), and the other scFv arm interacting with CD3ε subunit (expressed on all T cells). It was next examined whether such exemplary BsAb molecules can induce bidirectional killing (where crosslinking by the BsAbs induce activation of both “effector” and “target” T cells, thereby killing the crosslinked “effector” T cells (expressing any TCR; FIG. 2A). For example, the α-C1 crosslinking could activate TRBC1⁺ T cells and kill the conjugated TRBC2⁺ T cells. This would result in the killing of both TRBC1 and TRBC2 expressing T cells leading to the observed near complete T cell depletion (FIG. 1C). Similarly, α-V5 or α-V12 would result in bidirectional killing of T cells not expressing TCRV5-5 or V12. In contrast, BsAbs used in non-T cell cancer targeting strategies are directed against cancer-cell surface antigens and do not activate the target cancer cells, resulting in unidirectional killing. Three additional BsAbs were generated. These used the identical TRBV or TRBC scFvs described above, but substituted the α-CD3 scFv with an α-CD19 scFv (FIG. 2B and Table 1). This allowed us to test whether TRBC1, TRBV5-5 or TRBV12 engagement is sufficient for T cell activation and subsequent killing of CD19⁺ B cells (FIG. 2C). Conventional BsAb targeting CD19, in which an scFv against the CD3 is joined to the CD19-specific scFv, were used as a positive control. In the presence of any of the four BsAbs, co-culture of CD19⁺ target NALM6 B cells with normal T cells resulted in cytokine production including interferon gamma (IFNγ), interleukin 2 (IL-2), tumor necrosis factor alpha (TNFα) and interleukin 10 (IL-10) (FIG. 2D and FIG. 9B). It also resulted in the expression of T cell activation markers including CD25, inducible T cell co-stimulatory (ICOS), 4-1BB, and expression of exhaustion markers including lymphocyte-activation gene 3 (LAG-3), programmed death 1 (PD-1) (FIG. 9C) along with target NALM6 B cell killing (FIG. 2E), though at varying levels. The T cell cytokine production and NALM6 B cell cytotoxicity was dependent on NALM6 CD19 expression as NALM6 CD19 knock-out (FIG. 9A) abrogated these effects (FIG. 2D, FIG. 9B, and FIG. 2E). No difference in IFNγ production or NALM6 B cell cytotoxicity was observe between wild type NALM6 B cells and NALM6 B cells expressing lower levels of CD19 with the BsAbs (FIGS. 9A, D and E). To document the specificity of these effects, TRBC1⁺, TRBV5⁺, or TRBV12⁺ T cells were depleted from the T cell pool prior to co-culturing them with NALM6 B cells in the presence of the BsAbs. These depleted T cells remained inactivated as shown by their inability to generate IFNγ after co-culture with NALM6 B cells in the presence of α-V5-CD19 and α-V12-CD19 (FIG. 2F). Similarly, depleted T cells were unable to kill NALM6 B cells (FIG. 2H). Addition of α-V5-CD19 and α-V12-CD19 to TRBV5 and TRBV12 enriched human T cells restored IFNγ production and killing capacity to that of α-CD3-CD19 and α-C1-CD19 treatment conditions (FIGS. 2, G and I).

Exposure to α-CD3-CD19 and α-C1-CD19 resulted in considerably higher IFNγ levels and NALM6 cell cytotoxicity than exposure to α-V5-CD19 and α-V12-CD19. There are two potential reasons for this observation. First, approximately 35-45% human T cells express TRBC1 while 1.5% to 5% of normal T cells express TRBV5-5 or TRBV12 (FIG. 1C, D). The resulting effector to target (E:T ratio) is therefore much higher with α-CD3-CD19 and α-C1-CD19 than with α-V5-CD19 and α-V12-CD19. It was also possible that the T cell activating potentials of α-CD3 and α-TRBC1 scFvs are higher than those of α-TRBV5-5 or α-TRBV12 scFvs. This latter possibility was excluded by the demonstration that NALM6 cells co-cultured with TRBV5⁺ or TRBV12⁺ enriched T cells in the presence of α-V5 or α-V12 resulted in similar degrees of IFNγ production and cytotoxicity to those observed with α-CD3 and α-C1 BsAbs (FIGS. 2, G and I).

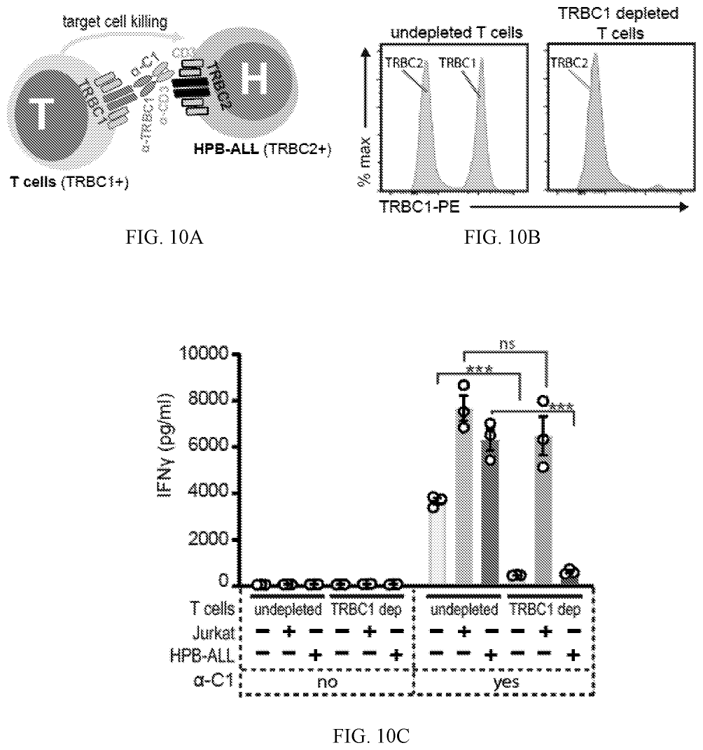

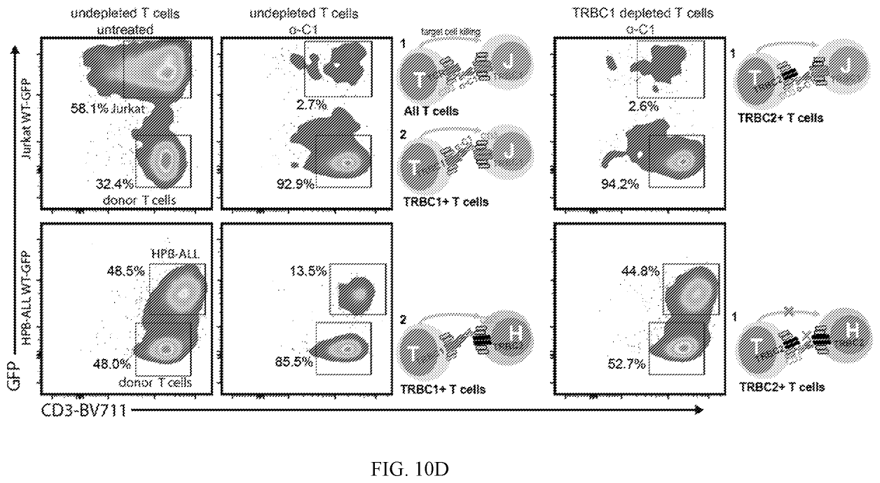

Similar experiments were performed that demonstrated α-C1 could mediate the death of clonal, neoplastic T cells expressing TRBC2 through bidirectional killing, even though these neoplastic T cells did not express TRBC1 (FIG. 10 A to D). α-C1 exposure induced IFNγ production against both TRBC1+ (Jurkat) and TRBC2+ (HPB-ALL) cells (FIG. 10C). Depletion of TRBC1+ T cell subset limited α-C1 induced IFNγ production against HPB-ALL cells (FIG. 10C). Flow cytometry analysis also showed α-C1 mediated HPB-ALL cell death with the use of undepleted T cells, while TRBC1+ T cell depletion reversed the effect (FIG. 10D). Depletion of normal TRBC1+ T cells did not affect α-C1 induced IFNγ response to Jurkat cells (FIG. 10C) or α-C1 mediated Jurkat cell killing (FIG. 10D) as α-C1 activated the remaining normal TRBC2+ T cells by CD3 crosslinking on these cells with TRBC1 on Jurkat cells. It was concluded that potent bidirectional killing can be mediated by BsAbs targeting TRBC1, TRBV5-5, or TRBV12. The reason that BsAbs targeting TRBV5-5 or V12 can be used to target T cells expressing those receptors without depleting the majority of T cells is because the number of TRBV5-5⁺ or TRBV12⁺ cells in normal T cell populations is much less than the number of TRBC1⁺ cells.

### TRBV-Directed BsAbs Induce T Cell Cytokine Responses Against Cancer Cells In Vitro

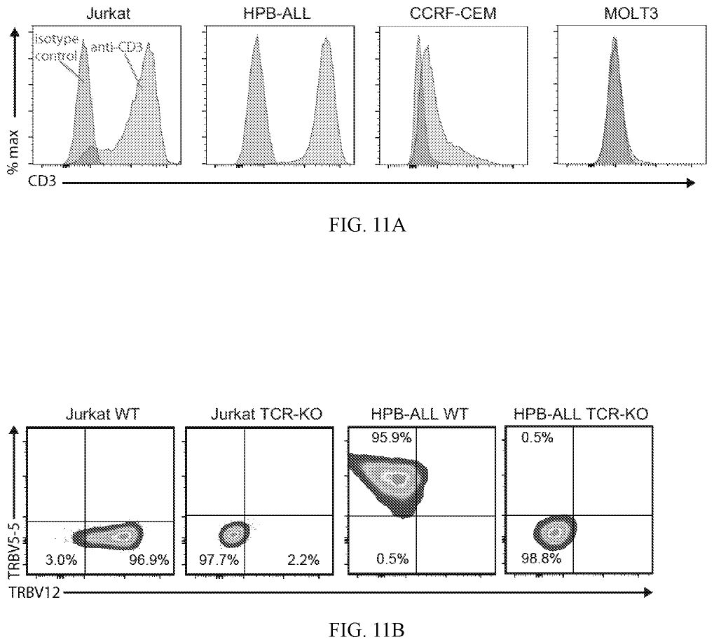

Human T cell cancer-derived cell lines have rearranged TCRβ genes and express clonal TRBVs. It was observed that T-ALL derived Jurkat, HPB-ALL and CCRF-CEM T cell lines retained cell-surface TCR expression as assessed with anti-CD3 antibodies, while MOLT3 cells did not (FIG. 11A). Jurkat and HPB-ALL cells also expressed surface TRBV12 and TRBV5-5, respectively (FIG. 11B). To assess the activity of BsAbs against T cell malignancies, normal T cells were co-cultured with T cell cancer cell lines in the presence or absence of different BsAbs.

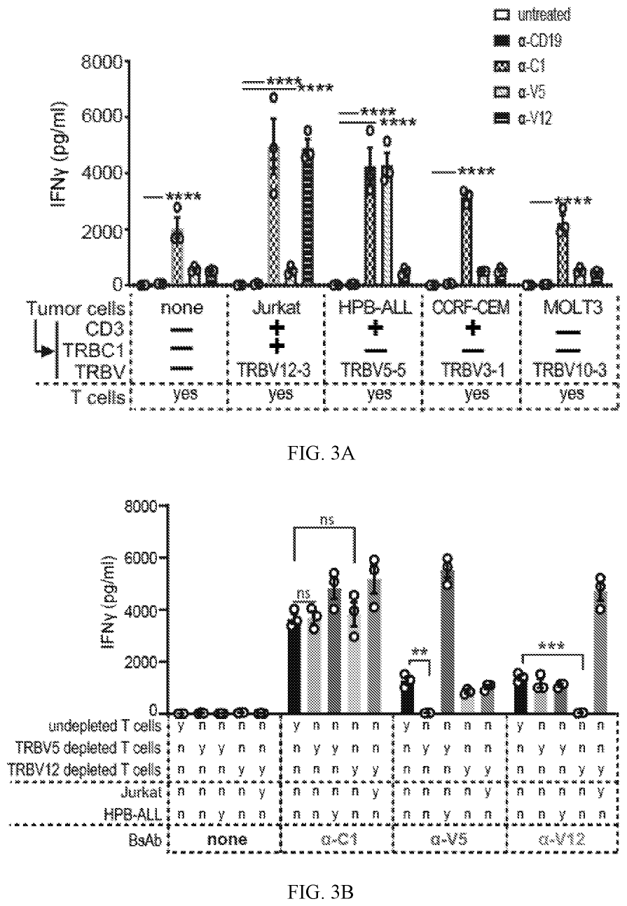

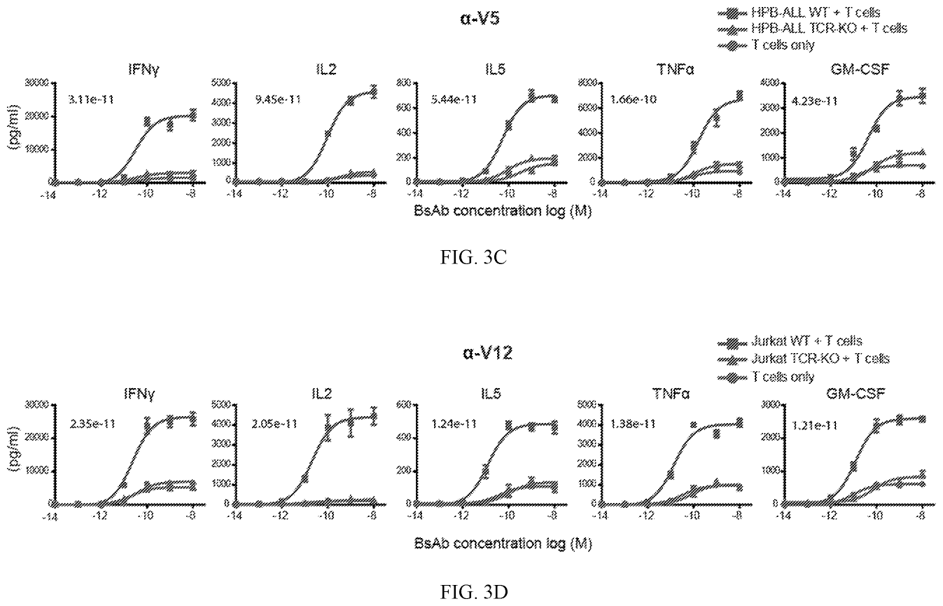

An increase in baseline IFNγ production, in the absence of any cancer cells, was noted after exposure to α-C1, and to a lesser degree with α-V5 and α-V12 (FIG. 3A). α-V5 and α-V12 increased T cell IFNγ secretion above baseline in the presence of HPB-ALL (TRBV5-5⁺) and Jurkat (TRBV12-3) cells, respectively. To confirm that the baseline IFNγ production in absence of target cancer cells was a result of the small percentage of TRBV5-5⁺ and TRBV12⁺ T cells present in the normal T cells, these cells were depleted before exposure to the BsAbs. TRBV5 and TRBV12 depleted T cells failed to produce IFNγ in response to α-V5 and α-V12 respectively (FIG. 3B). As a control for this experiment, it was shown that the depletion of TRBV5-5⁺ and TRBV12⁺ T cells did not affect IFNγ production in the presence of the α-C1 BsAb (FIG. 3B). Additionally, the TRBV5 depleted T cells secreted IFNγ when co-cultured with HPB-ALL (TRBV5-5⁺) cells in the presence of α-V5. Similarly TRBV12 depleted T cells co-cultured with Jurkat (TRBV12-3⁺) cells in the presence of α-V12 also secreted IFNγ. This also indicated that the TRBV depletion process itself did not result in loss of normal T cell function. The T cell activation via α-V5 and α-V12 was poly-functional, accompanied by the release of multiple cytokines including TNF-α, IL-2, interleukin-5 (IL-5) and granulocyte-macrophage colony-stimulating factor (GM-CSF) in addition to IFNγ (FIGS. 3, C and D).

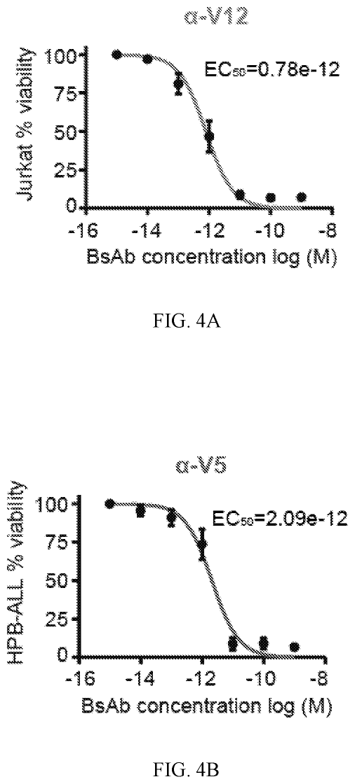

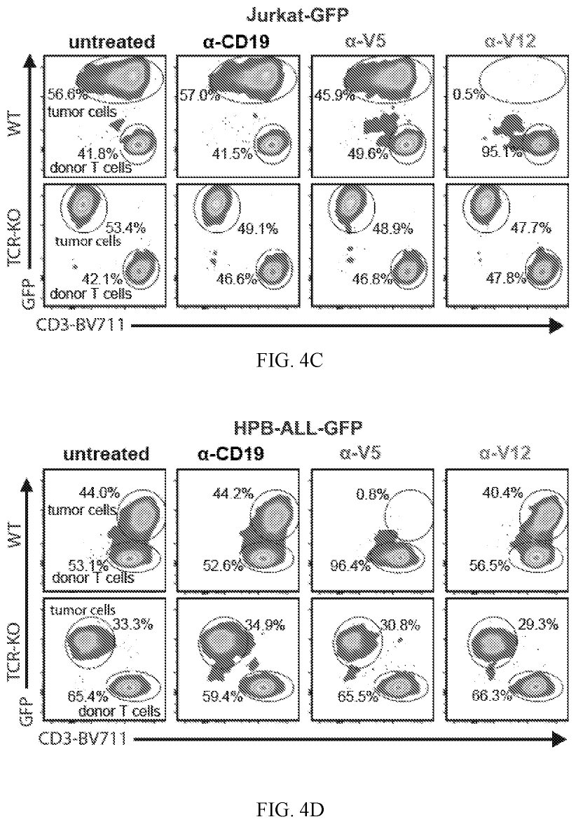

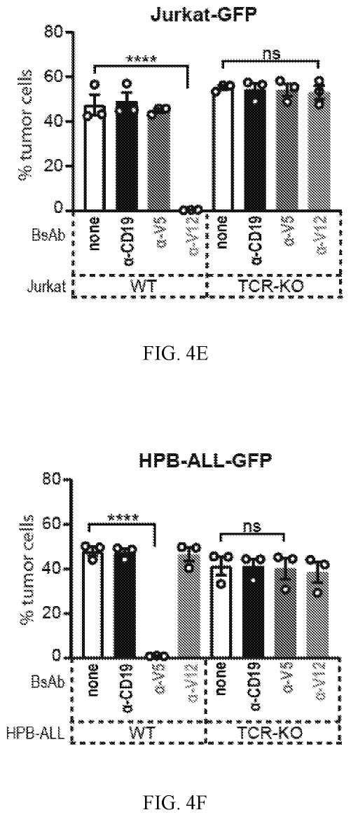

As a further control for specificity of these BsAbs, isogenic cancer cells were created using CRISPR-based disruption of TCR alpha and beta constant regions in both Jurkat and HPB-ALL cell lines. The resultant TCR knock-out (KO) was confirmed by loss of cell-surface TRBV12 or TRBV5-5 (FIG. 11B) and cell-surface CD3 expression (FIGS. 4, C and D). After TCR-KO, no significant increase in IFNγ or four other cytokines tested was observed upon co-culturing HPB-ALL cells with normal T cells in the presence of α-V5 (FIG. 3C). Similarly, little increase in cytokines was observed after co-culturing TCR-KO Jurkat cells with normal T cells in the presence of α-V12 (FIG. 3D).

### TRBV-Directed BsAbs Kill Cancer Cell Lines In Vitro

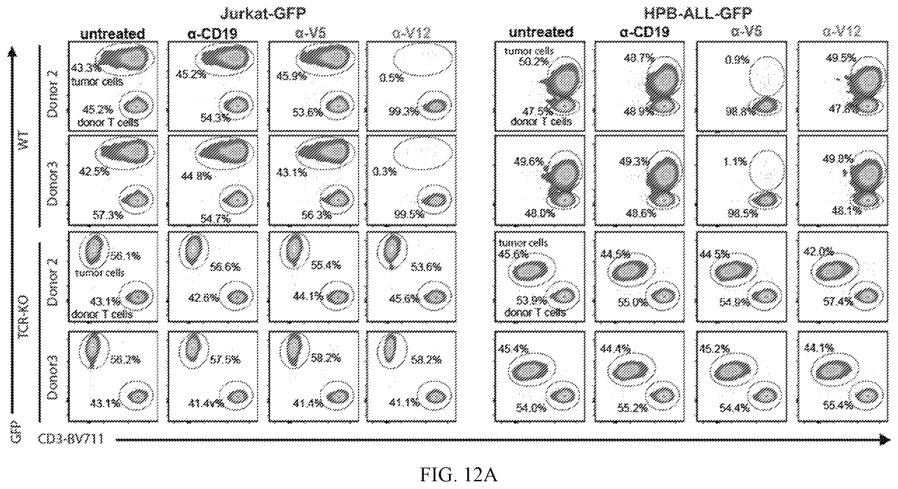

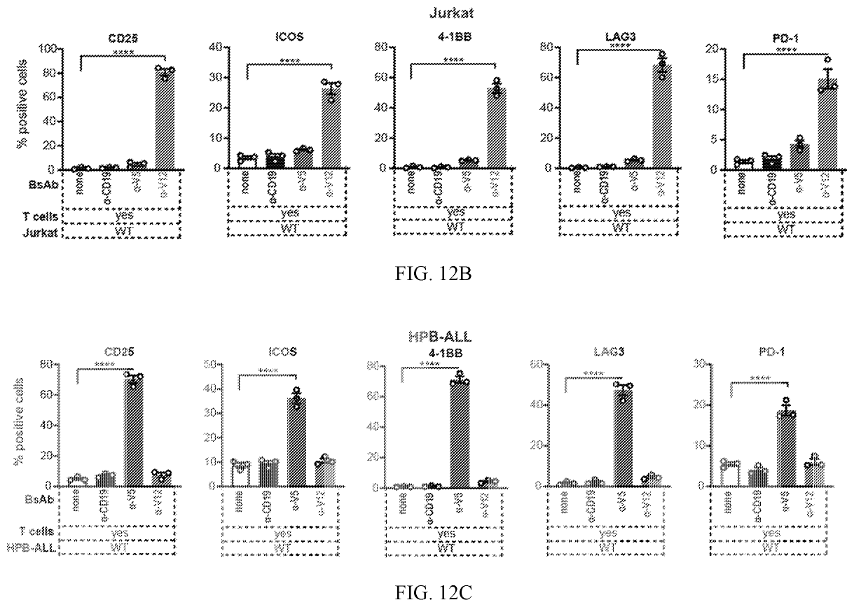

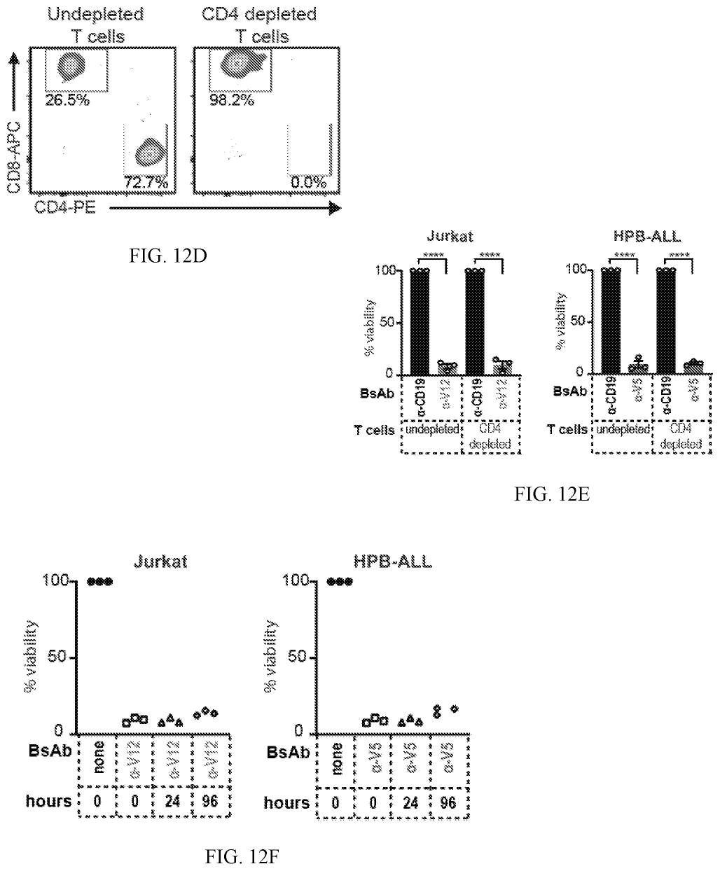

To assess cytotoxicity, normal human T cells were co-cultured with Jurkat or HPB-ALL cells in presence of increasing concentrations of α-V12 or α-V5 BsAbs (FIG. 4A, B). Almost complete Jurkat and HPB-ALL cytotoxicity was observed at 0.01 nM (0.57 ng/mL) concentration of both α-V12 and α-V5. Cancer cell lines were also engineered to express GFP. Jurkat cells expressing GFP were eliminated when co-cultured with normal T cells in the presence of α-V12 (FIG. 4C, E and FIG. 12A). Exposure to α-V12 and normal T cells had no significant effect on TCR-KO Jurkat cells (FIG. 4C, E and FIG. 12A). Similarly, HPB-ALL cells expressing GFP were eliminated when co-cultured with normal T cells in the presence of α-V5, and this elimination was abrogated in TCR-KO HPB-ALL cells (FIG. 4D, F and FIG. 12A). As another control for this experiment, it was shown that the α-CD19 BsAb had no effect on either Jurkat or HPB-ALL cells when incubated with normal T cells (FIG. 4, C to F and FIG. 12A). α-V12 also induced expression of activation and exhaustion markers on the normal human T cells in the presence of the target Jurkat cells (FIG. 12B). Similarly, α-V5 mediated expression of activation and exhaustion markers on the normal human T cells in the presence of the target HPB-ALL cells (FIG. 12C). No loss of α-V12 and α-V5 activity was observed with depletion of the CD4 helper T cells from the normal human T cells (FIG. 12D, E). Additionally, α-V12 and α-V5 cytotoxic function was preserved after incubation of the BsAbs with human serum for 96 hours prior to co-culture (FIG. 12F).

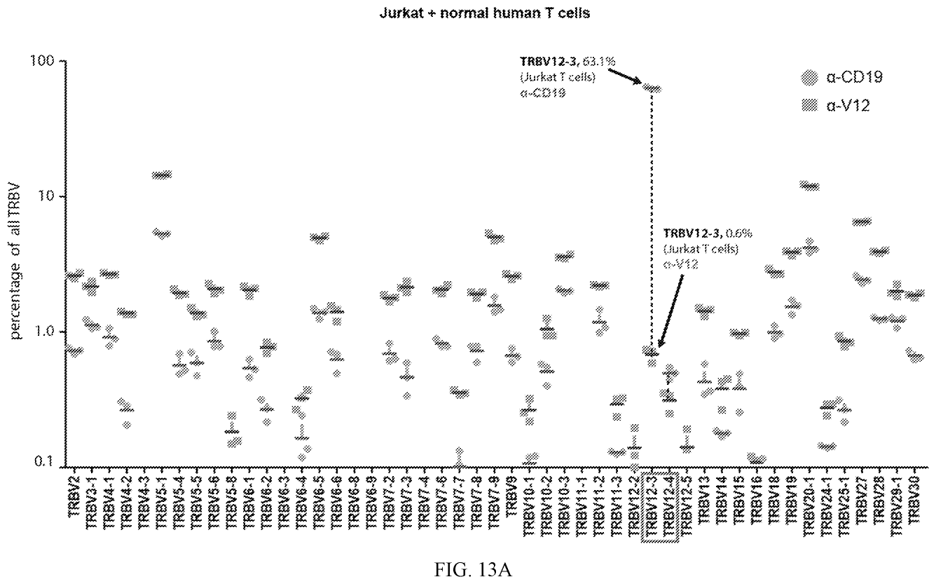

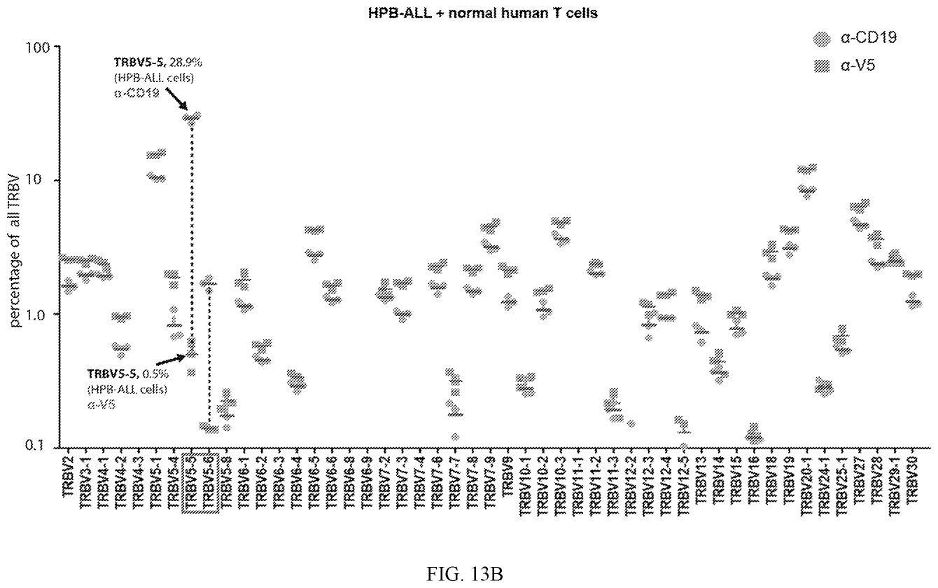

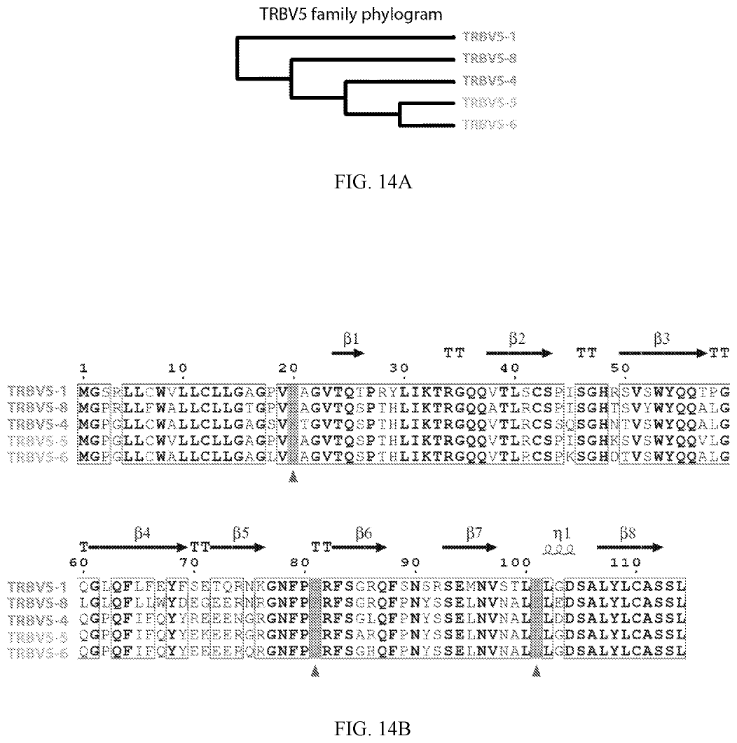

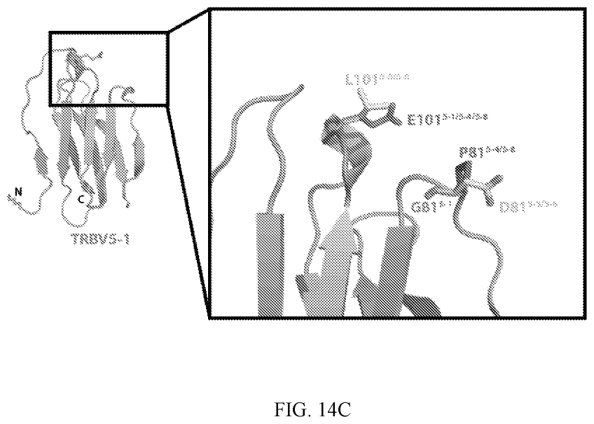

To determine whether α-V12 affects T cells expressing TRBV-families other than TRBV12, Jurkat cells and normal T cells were co-cultured in the presence of α-CD19 or α-V12, as noted above. TRBV gene sequencing was then performed to measure the percentage of TRBV depletion in surviving cells. A dramatic reduction (98.9%) in TRBV12-3 levels was detected after exposure to α-V12 compared with exposure to α-CD19 (FIG. 13A). The vast majority of the TRBV12-3 signal was of course derived from the Jurkat cells rather than the normal T cells. With the exception of TRBV12-4, which was reduced by 36.5%, other TRBV family members were unaffected (FIG. 13A). A similar analysis was performed with HPB-ALL cells. A dramatic reduction (98.3%) in TRBV5-5 levels was detected after exposure to α-V5 compared to that after exposure to α-CD19 (FIG. 13B). The vast majority of the TRBV5-5 signal was derived from the HPB-ALL cells rather than the normal T cells. Again with the exception of TRBV5-6, which was reduced by 91.6% (FIG. 13B), other TRBV family members remained unaffected. The sequence of the TRBV5-5 directed scFv used in the α-V5 BsAb was derived from an antibody originally developed against a TRBV5-5 antigen, thus it was not surprising that TRBV5-6-expressing T cells were affected by α-V5 exposure given that TRBV5-5 and TRBV5-6 are the most similar among TRBV5 family members (FIG. 14A, B). Sequence alignment of TRBV5 family members revealed that amino acid residues D20, D81 and L101 are common to both TRBV5-5 and TRBV5-6 but differ from other TRBV5 members, and the differences at residues 81 and 101 in the other TRBV5 family members also resulted in major charge differences (FIG. 14C).

### TRBV-Directed BsAb Kill Patient-Derived T-ALL Cells In Vitro

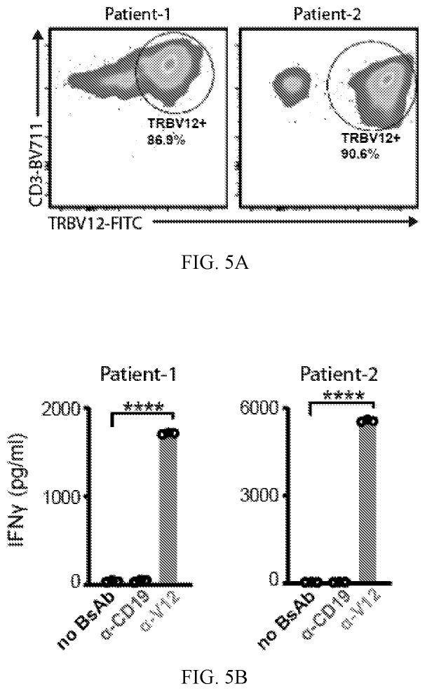

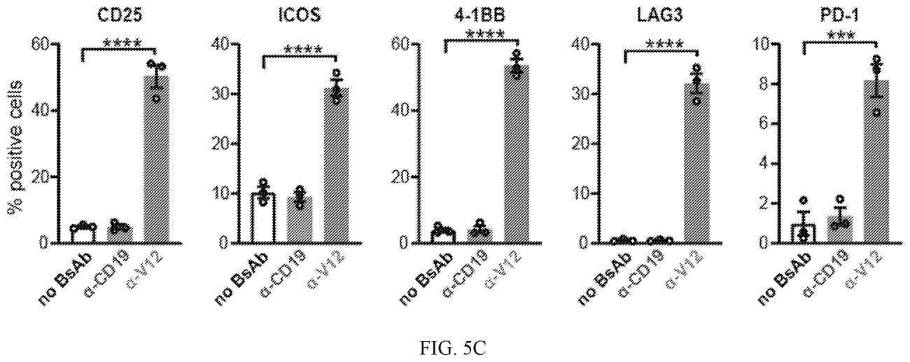

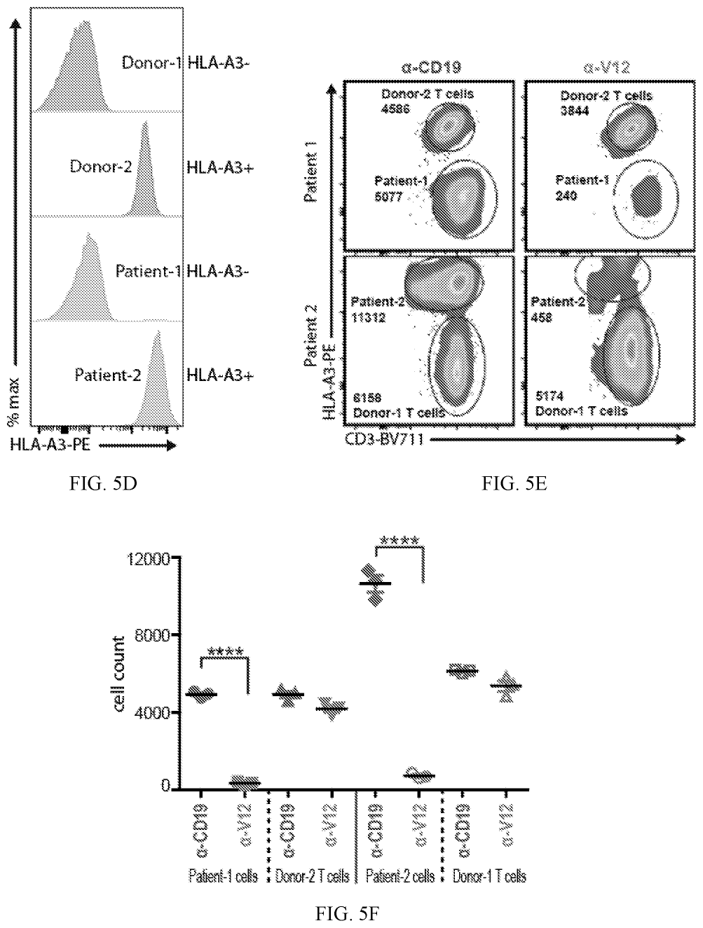

Primary malignant cells were collected from T-ALL patients. Flow cytometry identified two patients (Patients 1 and 2) with a substantial TRBV12⁺ population, suggesting presence of monoclonal cancer cells (FIG. 5A). T-ALL cells from patients 1 and 2 co-cultured with normal T cells in the presence of α-V12 led to significant IFNγ secretion (FIG. 5B), and expression of activation and exhaustion markers on normal human donor T cells (FIG. 5C). HLA-A3 expression was used to discriminate between the normal T cells derived from two healthy human donors and patient-derived T-ALL cells (FIG. 5D). Co-culture of Donor-2 T cells (HLA-A3⁺) with Patient-1 (HLA-A3⁻) malignant cells and α-V12 showed depletion of patient-derived malignant cells (FIG. 5E, F). Similarly, co-culture of Donor-1 (HLA-A3⁻) T cells with Patient-2 (HLA-A3⁺) malignant cells also showed depletion of the malignant cells. In both cases, the normal human T cells were relatively unaffected by exposure to α-V12 (FIG. 5E, F), as the low fraction of TRBV12-expressing cells among normal T cells (FIG. 1C, D).

### TRBV-Directed BsAbs Kill Cancer Cells In Vivo

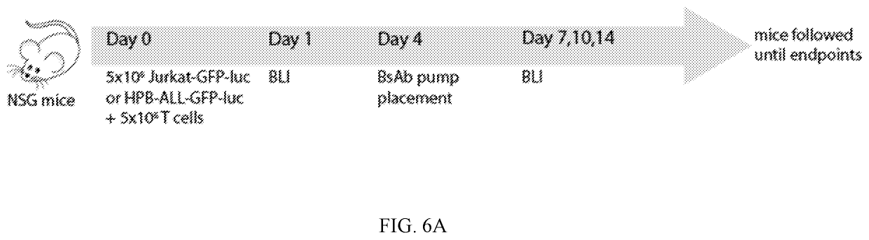

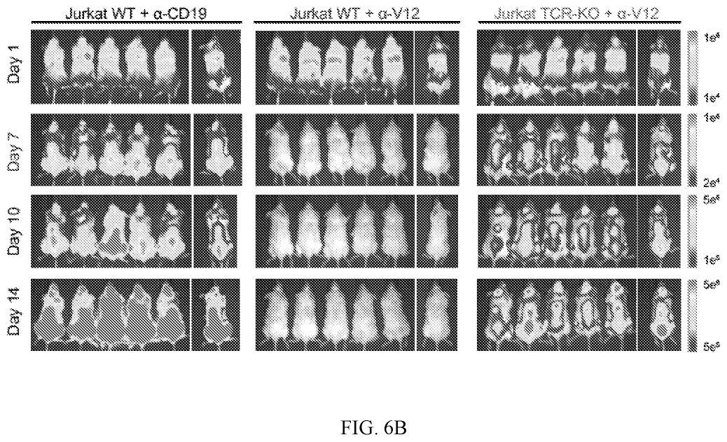

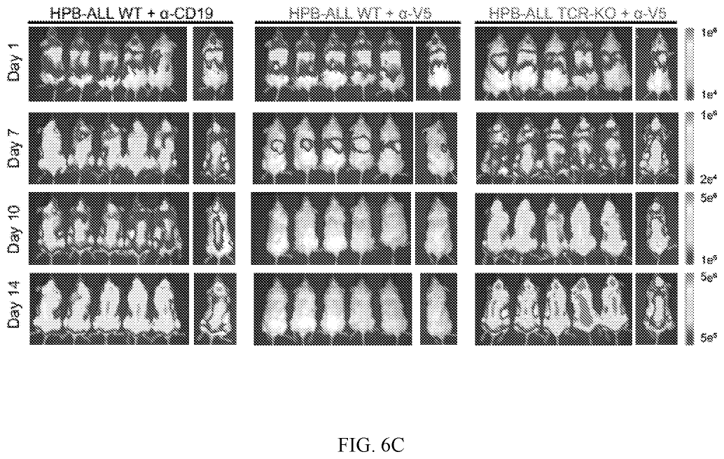

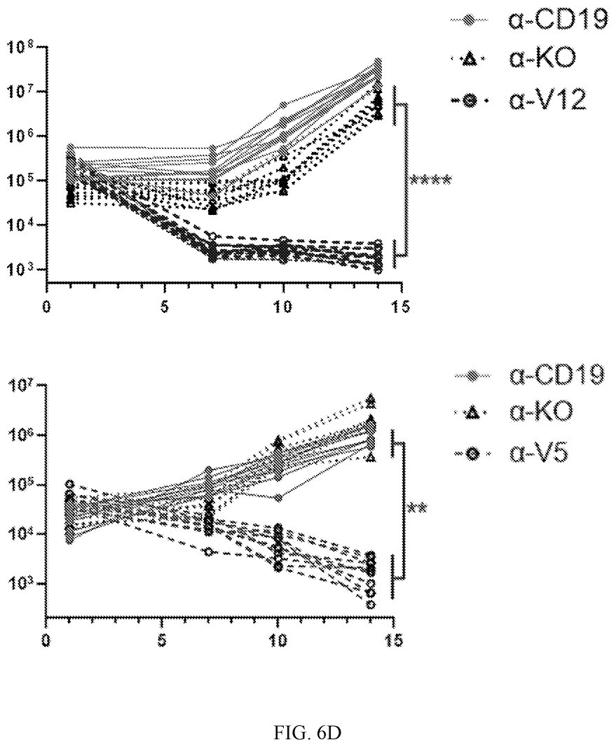

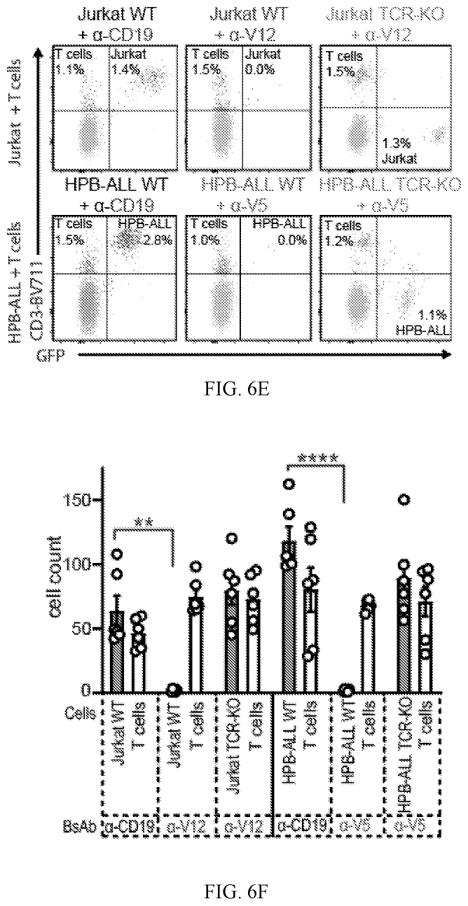

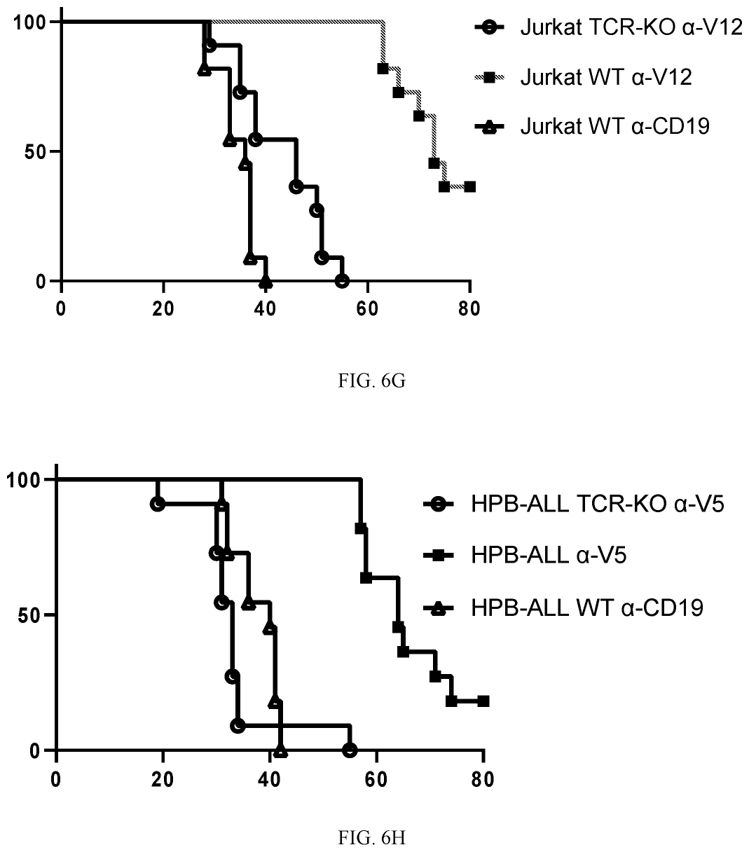

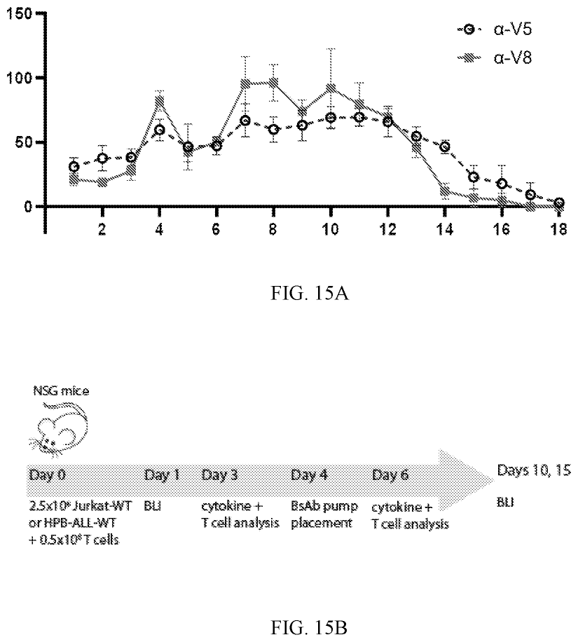

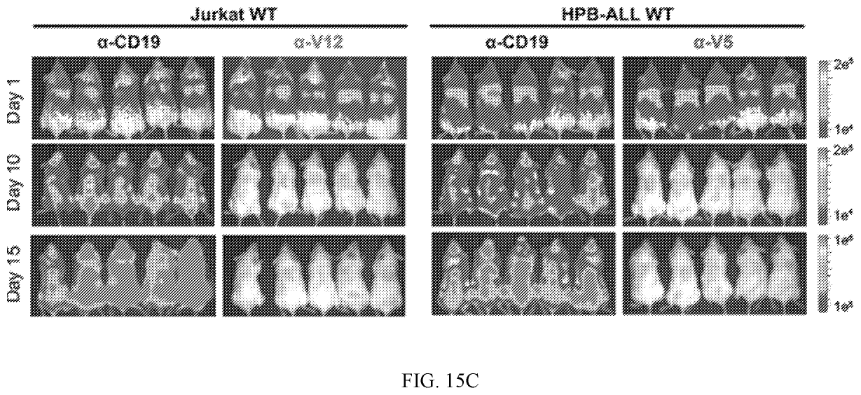

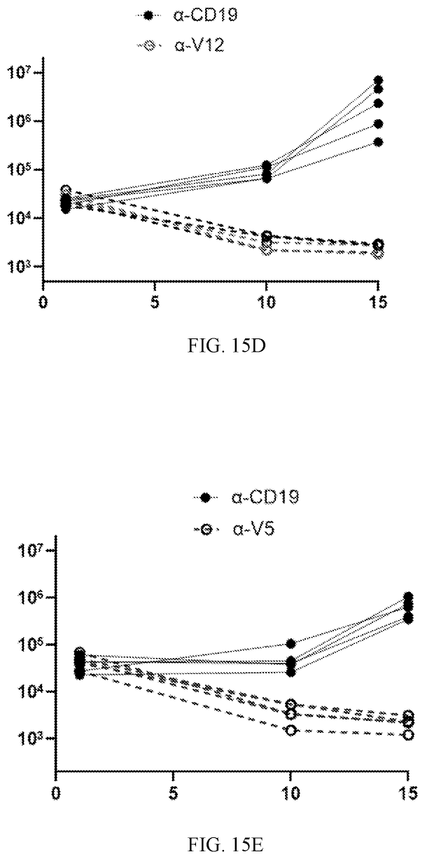

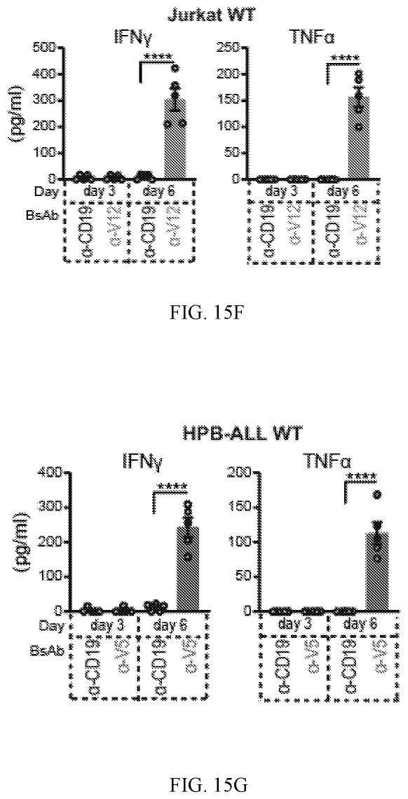

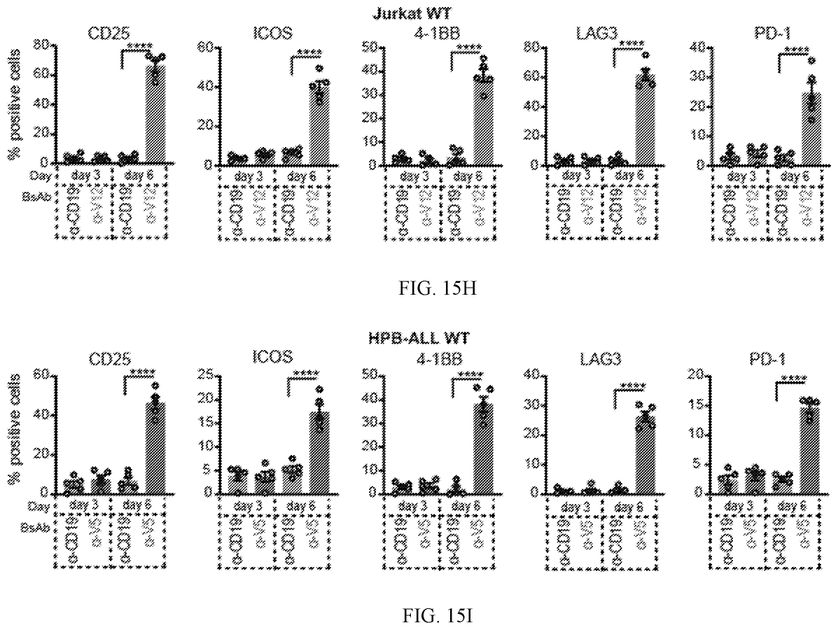

To assess efficacy in vivo, two disseminated xenograft models were established with luciferase-expressing Jurkat or HPB-ALL cancer cells injected intravenously into NOD.Cg-Prkdc^{scid}Il2rg^{tw1Wj1}/SzJ (NSG) mice (FIG. 6, A to C). All mice also received human T cells via intravenous injection. For the Jurkat model, the α-V12 BsAb was delivered through an intraperitoneal pump starting on day 4 after Jurkat and normal human T cell inoculation, when Jurkat cells were already widely disseminated (FIGS. 6, A and B). The intraperitoneal pumps were able to maintain significant serum concentrations of α-V12 and α-V5 for at least 2 weeks after implantation (FIG. 15A). Bioluminescence imaging (BLI) demonstrated marked tumor burden reduction in the mice treated with α-V12 (FIGS. 6, B and D). Two controls were used to document the specificity of this reduction, one for the BsAb and one for the cells. BsAb control: when mice harboring Jurkat cancers were treated with α-CD19 instead of α-V12, tumor burden was significantly higher as assessed by BLI (FIGS. 6, B and D). Cell control: when mice bearing disseminated cancers derived from Jurkat TCR-KO cells, tumor burden was markedly higher compared to mice bearing WT Jurkat cells after treatment with α-V12 (FIGS. 6, B and D). A second disseminated cancer model was used to document reproducibility of these in vivo results. The experimental approach was identical to that described for the Jurkat cell model, except that HPB-ALL cells were substituted for Jurkat cells and α-V5 was substituted for α-V12 (FIG. 6A, C). Again BLI demonstrated marked luminescence reduction in the mice treated with α-V5 (FIGS. 6, C and D). Analogous treatment with α-CD19 or mice bearing disseminated cancers derived from HPB-ALL TCR-KO cells also demonstrated a tumor growth similar to observations in the Jurkat model (FIGS. 6, C and D). Nineteen days after inoculation of cancer cells (15 days after initiating BsAb treatment) flow cytometric analysis of mouse blood revealed that all treatment groups retained normal human T cells (FIGS. 6, E and F). Additionally, there were abundant circulating Jurkat and HPB-ALL leukemia cells in α-CD19 treated mice (FIGS. 6, E and F). In striking contrast, α-V12 and α-V5 treated mice had a dramatic reduction in circulating leukemia cells in these experiments (FIGS. 6, E and F). This reduction in circulating leukemia cells was associated with a significant survival benefit in both α-V12 and α-V5 treated mice (FIGS. 6, G and H). α-CD19 treated mice developed hind-leg paralysis, leading to the need for euthanasia. Mice bearing Jurkat or HPP-ALL cancers that were treated with α-V12 or α-V5, respectively, did not develop hind-leg paralysis. These mice eventually died (FIGS. 6, G and H), and demonstrated typical graft-versus-host-disease (GVHD) features at time of death. However, if such an approach were used in humans, only the BsAbs, and not the T cells, would need to be administered. In human T cell cancers, an additional challenge is that the malignant T cells often outnumber the healthy effector T cells. To ascertain if a lower number of human effector T cells can sufficiently eradicate the T cell tumors in vivo, NSG mice were injected with 0.5×10⁶ human T cells along with 2.5×10⁶ tumor cells (Jurkat or HPB-ALL cells) (FIG. 15B). BLI demonstrated significant tumor burden reduction with both α-V12 and α-V5 treatment (FIG. 15C to E). α-V12 and α-V5 treatment also lead to elevated IFNγ and TNFα cytokine production (FIG. 15F, G) along with expression of T cell activation and exhaustion markers on normal human T cells (FIG. 15H, I).

Together these results demonstrate that TRBV-targeting BsAbs can deplete clonal cancerous T cells in vitro and in vivo while preserving the majority of normal T cells. Thus, TRBV-targeting BsAbs can be used to treat T cell cancers while avoiding treatment related immunosuppression.

Methods

### Cell Lines and Primary Human T Cells

Jurkat (Clone E6-1), CCRF-CEM, MOLT-3, (ATCC, Manassas, VA), HPB-ALL (DSMZ, Germany) and NALM6 cells were cultured in RPMI-1640 (ATCC, 30-2001) supplemented with 10% HyClone fetal bovine serum (FBS, GE Healthcare SH30071.03, Chicago, IL) and 1% Penicillin-Streptomycin (ThermoFisher Scientific, Waltham, MA). HEK293FT (ThermoFisher Scientific, Waltham, MA) was cultured in DMEM (ThermoFisher Scientific, 11995065) supplemented with 10% FBS, 2 mM GlutaMAX (ThermoFisher Scientific, 35050061), 0.1 mM MEM non-essential amino acids (ThermoFisher Scientific, 11140050), 1% Penicillin-Streptomycin, and 500 μg/mL Geneticin (ThermoFisher Scientific, 10131027). PBMCs were isolated from leukapheresis samples (Stem Cell Technologies, Vancouver, BC or Astarte Biologics, Bothell, WA) by Ficoll Paque Plus (GE Healthcare, GE17-1440-02) density gradient centrifugation. Human T cells were expanded from PBMCs either with addition of the anti-human CD3 antibody (clone OKT3, BioLegend, San Diego, CA, 317325) at 15 ng/mL, or with Human T-Activator CD3/CD28 Dynabeads (ThermoFisher Scientific, 11131D) for three days at a bead:cell ratio of 1:5. T cells were cultured in RPMI-1640 with 10% FBS, 1% Penicillin-Streptomycin, 100 IU/mL recombinant human IL-2 (aldesleukin, Prometheus Therapeutics and Diagnostics, San Diego, CA), and 5 ng/mL recombinant human IL-7 (BioLegend, 581906).

### Cell Staining, Flow Cytometry and Cell Sorting

Cells were suspended at 1×10⁶ cells/mL in flow stain buffer (PBS containing 0.5% BSA, 2 mM EDTA, 0.1% sodium azide) or flow sorting buffer (PBS containing 4% FBS) and incubated with appropriate antibodies for 30 minutes on ice. The antibodies used were: Brilliant Violet (BV)-711 anti-human CD3 (clone OKT3 BioLegend #317328); APC-anti-human CD45 (clone H130 BioLegend #304012); APC-anti-human CD19 (clone HIB19, Biolegend #302212), PE-anti-human CD4 (clone RPA-T4, Biolegend #300508), APC-anti-human CD8 (clone SKi, Biolegend #344722), PE-anti-human Cβ1 TCR (clone JOVI.1 BD #565776), PE-anti-human HLA-A3 (clone GAP.A3 BD #566605), PE-TCR v05.1 (clone ImmU157), PE-TCR Vβ5.3 (clone 3D11), PE-TCR V05.2 (clone 36213), FITC-TCR V08 (clone 56C5.2 Beckman Coulter), BV-421-anti-human CD25 (Biolegend #302630), APC-anti-human ICOS (Biolegend #313510), BV-750-anti-human-41BB (Biolegend #309844), BV-421-anti-human LAG3 (Biolegend #369314), and APC-anti-human-PD1 (Biolegend #329908). Stained cells were analyzed using an LSRII flow cytometer or sorted using BD FACSAria II (Becton Dickinson, Mansfield, MA). Gating on single live cells was performed with the use of viability dyes (LIVE/DEAD Fixable Near-IR, L10119; Aqua Dead Cell Stain Kit L34957 Invitrogen) and forward and side scatter characteristics. CellTrace Violet stain (ThermoFisher C34557) was performed per manufacturer instructions.

### TRBC, TRBV and CD4 Depletion or Enrichment

For TRBC1 T cell depletion, 1×10⁸ normal T cells were stained with PE-mouse anti-human Cβ1 TCR (final concentration 1 μg/ml) followed by PE negative (TRBC1 depleted) cell sorting. For TRBV5 T cell depletion or enrichment, 1×10⁸ normal T cells were stained with PE-TCR Vβ5.3 (binds TRBV5-5) and PE-TCR V35.2 (binds TRBV5-6), followed by PE negative (TRBV5 depleted) or PE positive (TRBV5 enriched) cell sorting. For TRBV12 T cell depletion or enrichment, 1×10⁸ normal T cells were stained with FITC-TCR V08 (binds TRBV12-3 and TRBV12-4 T cells), followed by FITC negative (TRBV12 depleted) or FITC positive (TRBV12 enriched) cell sorting. Alternatively, an EasySep PE Positive Selection Kit II (StemCell Technologies, 17684) was used for cell isolation. For CD4 T cell depletion, normal T cells were stained with PE-anti-human CD4 followed by EasySep PE Positive Selection Kit II used for CD4 negative (CD4 depleted) cell isolation.

### Bispecific Antibody Production, Purification and Stability

The α-TRBV5-5, α-TRBV12, α-TRBC1 and α-CD19 scFv sequences (Table 1) were synthesized by GeneArt (ThermoFisher Scientific). The scFv sequence was expressed as single chain diabody format using the following N- to C-terminus format: IL-2 signal sequence, anti-TRBV/TRBC/CD19 variable light chain (V_{L}), GGGGS linker (SEQ ID NO:25), α-CD3 variable heavy chain (V_{H}), (GGGGS)₃ linker (SEQ ID NO:26), α-CD3 V_{L}, GGGGS linker (SEQ ID NO:25), anti-TRBV/TRBC/CD19 V_{H}, and 6×HIS tag, and cloned into a pcDNA3.4 vector (ThermoFisher Scientific). BsAbs were expressed and purified by the JHU Eukaryotic Tissue Culture Core Facility or by GeneArt. For BsAb expression from JHU Eukaryotic Tissue Culture Core Facility. 1 mg of plasmid was transfected with polyethylenimine (PEI) at a ratio of 1:3 into a 1 L suspension culture of HEK293F cells at a density of 2×10⁶ cells/mL. Newly transfected HEK293F cells were grown in Freestyle293 expression media for 5 days at 37° C., 170 rpm, and 5% C02. Subsequently, the media was harvested by centrifugation, filtered with a 0.22 μm unit, and the BsAb was purified using Nickel affinity chromatography. For this purpose, 2 mL of Ni-NTA His-Bind (Millipore Sigma, 70666-6) resin was added to the filtered supernatant and incubated at 4° C. overnight in an orbital shaker. The supernatant-resin mixture was captured by a gravity chromatography column (Econo-Pac Chromatography Columns 7321010, Bio-Rad, Hercules, CA) and washed with 20 mM imidazole (GE Healthcare, 45-000-007) in phosphate buffered saline (PBS). The desired BsAb was eluted with 500 mM imidazole, and desalted into PBS using a 7k MWCO Zeba Spin desalting column (ThermoFisher Scientific, 89883). Proteins were quantified via SDS-PAGE gel electrophoresis (Mini-PROTEAN TGX Stain-Free Precast Gel, Bio-Rad, 4568095) and/or using BCA protein assay (Pierce, ThermoFisher, 23225). Proteins were stored at −80° C. with 7% glycerol. Alternatively, BsAbs were produced by GeneArt in Expi293s, and purified with a HisTrap column (GE Healthcare, 17-5255-01) followed by size exclusion chromatography using a HiLoad Superdex 200 26/600 column (GE Healthcare, 28989336). Analytic chromatography was performed using TSKgel G3000SWxl column (TOSOH Bioscience) using a running buffer of 50 mM sodium phosphate and 300 mM sodium chloride at pH 7, at a flow rate of 1.0 mL/minute. BsAb Coomassie blue stain (ThermoFisher Scientific, 20278) and anti-histidine western blot were performed using anti-6×-His tag antibody (ThermoFisher Scientific, MA1-21315) by GeneArt.

Thermal stability of the α-V12 and α-V5 BsAbs were evaluated by a differential scanning fluorimetry which monitors the fluorescence of a dye that binds to the hydrophobic region of a protein as it becomes exposed upon temperature induced denaturation. Reaction mixtures (20 μL) were set up in a white low-profile 96-well, unskirted polymerase chain reaction plates (Bio-Rad, MLL9651) by mixing 2 μL of purified α-V12 or α-V5 BsAb at a concentration of 1 mg/mL with 2 μL of 50×SYPRO orange dye (Invitrogen S6650) in pH 7.4 phosphate buffered saline (PBS, Gibco, 10010023). Plates were sealed with an optical transparent film and centrifuged for 1000×g for 30 seconds. Thermal scanning was performed from 25 to 100° C. (1° C./minute temperature gradient) using a CFX9 Connect real-time polymerase chain reaction instrument (Bio-Rad). Protein unfolding/melting temperature (Tₘ) was calculated from the maximum value of the negative first derivative of the melt curve using CFX Manager Software (Bio-Rad). Serum stability was assessed by incubating the BsAbs with human serum (Millipore Sigma #H4522) at 0.05 μg/mL concentration in a 37° C. incubator for 0, 24, and 96 hours. At each time point, the human serum BsAb mixture was collected and frozen at −80° C. until BsAb functional analysis by a co-culture assay.

### CRISPR Gene Editing

The Alt-R CRISPR system (Integrated DNA Technologies, Coralville, IA) was used to generate TCR knock-out Jurkat and HPB-ALL cell lines as well as CD19 knock-out and CD19 low expressing NALM6 clones. For the knockout of TCRs, Alt-R CRISPR Cas9 crRNAs targeting the TRA constant region (AGAGTCTCTCAGCTGGTACA; SEQ ID NO:31), TRB constant region (AGAAGGTGGCCGAGACCCTC; SEQ ID NO:32), and Alt-R CRISPR-Cas9 tracrRNA (IDT, 1072533) were re-suspended at 100 μM in Nuclease-Free Duplex Buffer. The crRNAs and tracrRNA were duplexed at a 1:1 molar ratio for 5 minutes at 95° C. followed by cooling down slowly to room temperature according to the manufacturer's instructions. The duplexed RNA was then mixed with Cas9 Nuclease at a 1.2:1 molar ratio for 15 minutes. A total of 40 pmoles of the Cas9 RNP complexed with gRNA were mixed with 500,000 cells in 20 μL of OptiMEM (ThermoFisher, 51985091). This mixture was loaded into a 0.1 cm cuvette (Bio-Rad) and electroporated at 90 V for 15 milliseconds using an ECM 2001 (BTX, Holliston, MA). Cells were immediately transferred to the complete growth medium and cultured for 7 days. Single cell clones were established by limiting dilution and genomic DNA isolated using a Quick-DNA 96 Kit (Zymo Research, Irvine, CA, D3010). Regions flanking the CRISPR cut sites were PCR amplified (TCRα forward primer: GCCTAAGTTGGGGAGACCAC (SEQ ID NO:33), reverse primer: GAAGCAAGGAAACAGCCTGC (SEQ ID NO:34); TCRβ forward primer: TCGCTGTGTTTGAGCCATCAGA (SEQ ID NO:35), reverse primer: ATGAACCACAGGTGCCCAATTC (SEQ ID NO:36) and Sanger sequenced to select for TCRα-/β-clones. TRA and TRB chain gene disruption was confirmed by the loss of surface CD3 expression.

To generate CD19 knock-out and CD19 low NALM6 clones, an Alt-R CRISPR sgRNA (CGAGGAACCTCTAGTGGTGA; SEQ ID NO:37) was complexed with Cas9 Nuclease (IDT) at a 2:1 molar ratio for 15 minutes at room temperature. Then, 50 pmoles of Cas9 RNP were mixed with 200,000 NALM6 cells re-suspended in 20 μL SF buffer (Lonza) and electroporated with a 4D Nucleofector X-unit (Lonza) in 16-well cuvette strips using pulse code CV-104. The cells were cultured in complete growth media for 7 days prior to dilutional plating to select individual clones. The cell surface CD19 levels of clones were characterized by flow cytometry staining with anti-human CD19 antibody.

### Retroviral Transduction

Non-tissue culture treated plates were coated with 100 μL RetroNectin (Clontech Takara, Mountain View, CA, T202) in PBS at 20 μg/mL overnight at 4° C., then blocked with 10% FBS for 1 hour at room temperature. Retrovirus (RediFect Red-FLuc-GFP, PerkinElmer CLS960003) and 2×10⁵ target cells were added to each well and centrifuged at 2000×g for 1 hour at 20° C. Plates were incubated for two days at 37° C., after which cells were expanded to a 6-well plate. Transduced cells were isolated by FACS (BD FACSAria II) based on GFP expression.

### TCR Sequencing

Total RNA was isolated from samples with Qiagen AllPrep DNA/RNA Micro kits (Qiagen, 80284). RNA quality was validated using an Agilent TapeStation system. TCR sequencing libraries were prepared using a 5′ RACE (rapid amplification of cDNA ends) method consisting of a cDNA synthesis step followed by two PCR steps with gene-specific primers for the TCRβ constant region. Libraries were sequenced using an Illumina MiSeq platform. Reads were analyzed with MIGEC, MiXCR, and VDJtools. Frequencies of clonotypes were calculated as the proportion of UIDs (unique molecular identifier barcodes) representing the clonotype among all UIDs in the sample. The following non-functional TRBVs (listed as pseudogenes or as open reading frames in IMGT) were excluded from analysis; TRBV1, TRBV3-2, TRBV5-2, TRBV5-3, TRBV5-7, TRBV6-7, TRBV7-1, TRBV7-5, TRBV8, TRBV12-1, TRBV12-2, TRBV21, TRBV22, TRBV23-1, TRBV26.

### TRBV Sequence and Structural Alignment

The structures of PDB ID 5BRZ, 6EH5, 4P4K, and 4QRR were structurally aligned and residues 2-95 were extracted from 5BRZ, corresponding to the TCR beta variable region of TRBV 5.1. To model TRBV 5.4, 5.5, 5.6 and 5.8, in silico mutations were performed at positions 81 and 101 using Coot. Figures were rendered in PyMOL (v2.2.3, Schrödinger, LLC, New York, NY). Alignment of relevant TRBV sequences was performed using ClustalOmega and displayed using Espript.

### Co-Cultures

Co-cultures were set up using 96-well flat-bottom tissue culture treated plates, with each well containing 5×10⁴ normal human T cells (effector cells), 5×10⁴ target cells (indicated in text) and BsAbs (concentration specified in text) in a total 100 μL volume RPMI media. The co-cultures were incubated for 17 hours at 37° C. The supernatant was assayed for cytokines using a Human IFN-gamma Quantikine ELISA Kit (R&D Systems, Minneapolis, MN, SIF50C), Human IL-2 Quantikine ELISA Kit (R&D Systems, S2050), Human TNF-alpha Quantikine ELISA Kit (R&D Systems, STA00D), Human IL-10 Quantikine ELISA Kit (R&D Systems, S1000B), or a Luminex assay (13-plex-Immunology Multiplex Assay, Millipore Sigma, USA, HMHEMAG-34K) performed on the Bio-Plex 200 system (Bio-Rad). For luciferase expressing target cells, cell viability was assayed by the Steady-Glo luciferase assay (E2510, Promega, Madison, WI), per manufacturer's instructions. Viability was calculated as the ratio of luminescence signal to the no antibody or control antibody condition: (antibody well luminescence)/(no antibody or control antibody well luminescence). Alternatively, tumor cells were quantified by flow cytometry based GFP expression (for GFP-expressing tumor cell lines) or distinct HLA expression (for patient-derived tumor cells). For experiments to detect effects of BsAbs on healthy T cells in the absence of target tumor cells, 1×10⁶ normal human T cells was incubated with the BsAbs (concentration specified in text) in a total 1 mL volume RPMI media, and incubated for 17 hours at 37° C. Viable T cells were quantified by counting trypan blue stained cells on a hemocytometer.

### T-ALL Patient Sample Collection

T cell cancer patient samples were collected in accordance with the Johns Hopkins Institutional Review Board (IRB: NA_00028682, and NA_00028682) approved Hematologic Malignancy Cell Bank Protocol (J0969) or the Johns Hopkins Pediatric Leukemia Bank Protocol (J0968).

### Animal Experiments

Six to eight week old female NOD.Cg-Prkdc^{scid}Il2rg^{tm1wj1}/SzJ (NSG) mice acquired from the Johns Hopkins Sidney Kimmel Comprehensive Cancer Center Animal Resources facility were maintained according to JHU Animal Care and Use Committee approved research protocol MO18M79. Cancer cell lines and human T cells were injected via the tail vein. Two-week micro-osmotic pumps (Model 1002, ALZET, Cupertino, CA) were filled with BsAb as indicated in the text using a 30G needle. Pumps were placed in the peritoneal space of each mouse using sterile surgical technique. For survival studies, animals were followed until day 80 or sacrificed when exhibited evidence of paralysis or GVHD (hunched posture, fur ruffling, scaling or denuded skin, reduced activity). Mouse bioluminescence was measured using the IVIS system (PerkinElmer, USA). Prior to imaging, mice were anesthetized using inhaled isoflurane in an induction chamber. Following induction, mice received intraperitoneal injection of luciferin (150 μl, RediJect D-Luciferin Ultra Bioluminescent Substrate, PerkinElmer, 770505), and were placed in the imaging chamber after 5 minutes. Luminescence images were analyzed using Living Image software (version 4.7.2, PerkinElmer). For flow-based detection of tumor cells and normal human T cells from mouse blood, 100 μL blood was collected in EDTA treated microvettes (Sarstedt Inc, NC9299309) by mouse cheek bleed, followed by 10 minutes incubation with 1 mL ACK lysis buffer (Quality Biological, 118-156-721), resuspension in flow stain buffer with mouse and human TrueStain FcX Fc receptor blocking solutions (BioLegend, 101320, 422302) and cell-surface staining antibodies. 10 μL of counting beads (Precision Count Beads, BioLegend, 424902) were added to equal volume (300 μL) of cell suspension in each tube. The number of tumor cells (GFP⁺, CD3⁺) or T cells (GFP⁻, CD3⁺) were counted based on acquisition of 500 beads for each sample. For cytokine and BsAb detection, blood from mice was collected in eppendorf tubes and allowed to clot for 30 minutes at room temperature, followed by centrifugation at 1000×g for 5 minutes at 4° C. Serum was collected and stored at −80° C. until cytokine (per manufacturer instructions) or BsAb ELISA. For BsAb ELISA, mouse serum was incubated in biotinylated recombinant human CD3 epsilon & CD3 delta (Acro Biosystems, DE, USA, #CDD-H52W4) coated streptavidin plates (R&D Systems, #CP004), followed by detection using HRP conjugated anti-human kappa light chain antibody (ThermoFisher Scientific, #A18853).

### Statistical Analyses

Mean±standard error of mean was used to summarize the data. The Student's t-test was used to compare differences in means between two samples for normally distributed variables. For three or more groups, one-way ANOVA with Tukey's multiple comparison test (when comparing all groups) or Dunnett's test (when comparing test groups to one control group) or Sidak test (when comparing two select groups) were used, with α=0.05. The Kaplan-Meier method was utilized to generate median survival, and the hazard ratios estimated by log-rank test. Prism version 8.0 software (GraphPad, La Jolla, CA) was used for statistical analysis and graph production.

### SEQUENCE LISTING

SEQUENCE LISTING

SEQ ID NO: 1
anti-TRBV5-5 V_{L} CDR1
CSASQGISNYLN
SEQ ID NO: 2
anti-TRBV5-5 V_{L} CDR2
TSSLHSGV
SEQ ID NO: 3
anti-TRBV5-5 V_{L} CDR3
QQYSKLPRT
SEQ ID NO: 4
anti-TRBV5-5 V_{H} CDR1
AYGVN
SEQ ID NO: 5
anti-TRBV5-5 V_{H} CDR2
WGDGNTDYNSALK
SEQ ID NO: 6
anti-TRBV5-5 V_{H} CDR3
ATLYAMDY
SEQ ID NO: 7
anti-TRBV5-5 V_{L}
DIQMTQTTSSLSASLGDRVTITCSASQGISNYLNWYQQKPDGTVKLLIYYTSSLHSGV
PSRFSGSGSGTDYSLTISNLEPEDIATYYCQQYSKLPRTFGGGTKVEIK
SEQ ID NO: 8
anti-TRBV5-5 V_{H}
QVQLKESGPGLVAPSQSLSITCTVSGFSLTAYGVNWVRQPPGKGLEWLGMIWGDGN
TDYNSALKSRLSISKDNSKSQVFLKMNSLQTDDTARYYCARDRVTATLYAMDYWG
QGTSVTVSS
SEQ ID NO: 9
anti-TRBV12 V_{L} CDR1
CRASSSVNYIYW
SEQ ID NO: 10
anti-TRBV12 V_{L} CDR2
YTSNLAPGVP
SEQ ID NO: 11
anti-TRBV12 V_{L} CDR3
QQFTSSPFT
SEQ ID NO: 12
anti-TRBV12 V_{H} CDR1
NFGMH
SEQ ID NO: 13
anti-TRBV12 V_{H} CDR2
YISSGSSTIYYADTLKG
SEQ ID NO: 14
anti-TRBV12 V_{H} CDR3
RGEGAMDY
SEQ ID NO: 15
anti-TRBV12 V_{L}
ENVLTQSPAIMSASLGEKVTMSCRASSSVNYIYWYQQKSDASPKLWIYYTSNLAPG
VPTRFSGSGSGNSYSLTISSMEGEDAATYYCQQFTSSPFTFGQGTKLEIK
SEQ ID NO: 16
anti-TRBV12 V_{H}
DVQLVESGGGLVQPGGSRKLSCAASGFTFSNFGMHWVRQAPDKGLEWVAYISSGSS
TIYYADTLKGRFTISRDNPKNTLFLQMTSLRSEDTAMYYCARRGEGAMDYWGQGTS
VTVSS
SEQ ID NO: 17
anti-CD3 V_{L} CDR1
RASQDIRNYLN
SEQ ID NO: 18
anti-CD3 V_{L} CDR2
(Y)YTSRLHS (with the first Y being optional)
SEQ ID NO: 19
anti-CD3 V_{L} CDR3
QQGNTLPWT
SEQ ID NO: 20
anti-CD3 V_{H} CDR1
GYTMN
SEQ ID NO: 21
anti-CD3 V_{H} CDR2
LINPYKGVSTYNQKFKD
SEQ ID NO: 22
anti-CD3 V_{H} CDR3
SGYYGDSDWYFDV
SEQ ID NO: 23
anti-CD3 UCHT1 V_{L}
DIQMTQTTSSLSASLGDRVTISCRASQDIRNYLNWYQQKPDGTVKLLIYYTSRLHSG
VPSKFSGSGSGTDYSLTISNLEQEDIATYFCQQGNTLPWTFAGGTKLEIK
SEQ ID NO: 24
anti-CD3 UCHT1 V_{H}
EVQLQQSGPELVKPGASMKISCKASGYSFTGYTMNWVKQSHGKNLEWMGLINPYK
GVSTYNQKFKDKATLTVDKSSSTAYMELLSLTSEDSAVYYCARSGYYGDSDWYFD
VWGAGTTVTVSS
SEQ ID NO: 25
polypeptide linker
GGGGS
SEQ ID NO: 26
polypeptide linker
(GGGGS)3
SEQ ID NO: 27
anti-TRBC1 V_{L}
DVVMTQSPLSLPVSLGDQASISCRSSQRLVHSNGNTYLHWYLQKPGQSPKLLIYRVS
NRFPGVPDRFSGSGSGTDFTLKISRVEAEDLGIYFCSQSTHVPYTFGGGTKLEIKR
SEQ ID NO: 28
anti-TRBC1 V_{H}
EVRLQQSGPDLIKPGASVKMSCKASGYTFTGYVMHWVKQRPGQGLEWIGFINPYND
DIQSNERFRGKATLTSDKSSTTAYMELSSLTSEDSAVYYCARGAGYNFDGAYRFFDF
WGQGTTLTVSS
SEQ ID NO: 29
anti-CD19 V_{L}
DIQLTQSPASLAVSLGQRATISCKASQSVDYDGDSYLNWYQQIPGQPPKLLIYDASNL
VSGIPPRFSGSGSGTDFTLNIHPVEKVDAATYHCQQSTEDPWTFGGGTKLEIK
SEQ ID NO: 30
anti-CD19 V_{H}
QVQLQQSGAELVRPGSSVKISCKASGYAFSSYWMNWVKQRPGQGLEWIGQIWPGD
GDTNYNGKFKGKATLTADESSSTAYMQLSSLASEDSAVYFCARRETTTVGRYYYA
MDYWGQGTTVTVSS
SEQ ID NO: 31
crRNA targeting the TRA constant region
AGAGTCTCTCAGCTGGTACA
SEQ ID NO: 32
crRNA targeting the TRB constant region
AGAAGGTGGCCGAGACCCTC
SEQ ID NO: 33
PCR primer
GCCTAAGTTGGGGAGACCAC
SEQ ID NO: 34
PCR primer
GAAGCAAGGAAACAGCCTGC
SEQ ID NO: 35
PCR primer
TCGCTGTGTTTGAGCCATCAGA
SEQ ID NO: 36
PCR primer
ATGAACCACAGGTGCCCAATTC
SEQ ID NO: 37
sgRNA targeting CD19
CGAGGAACCTCTAGTGGTGA
SEQ ID NO: 38
anti-TRBV5-5 V_{L}
DIQMTQSPSSLSASVGDRVTITCSASQGISNYLNWYQQTPGKAPKLLIYYTSSLHSGV
PSRFSGSGSGTDYTFTISSLOPEDIATYYCQQYSKLPRTFGQGTKLQIT
SEQ ID NO: 39
anti-TRBV5-5 V_{H}
QVQLQESGPGLVRPSQSLSITCTVSGFSLTAYGVNWVRQPPGRGLEWLGMIWGDGN
TDYNSALKSRVTMLKDTSKNQFSLRLSSVTAADTAVYYCARDRVTATLYAMDYW
GQGSLVTVSS
SEQ ID NO: 40
anti-TRBV12 V_{L}
DIQMTTQSPSSLSASVGDRVTITCRASSSVNYIYWYQQTPGKAPKLLIYYTSNLAPGV
PSRFSGSGSGTDYTFTISSLQPEDITYYCQQFTSSPFTFGSGTKLQIT
SEQ ID NO: 41
anti-TRBV12 V_{H}
EVOLVESGGGVVQPGGSRKLSCSSSGFTFSNFGMHWVRQAPGKGLEWVAYISSGSS
TIYYADTLKGRFTISRDNSKNTLFLQMDSLRPEDTGVYFCARRGEGAMDYWGQGTS
VTVSS
SEQ ID NO: 42
anti-CD3 UCHT1v9 V_{L}
DIQMTQSPSSLSASVGDRVTITCRASQDIRNYLNWYQQKPGKAPKLLIYYTSRLESG
VPSRFSGSGSGTDYTLTISSLQPEDFATYYCQQGNTLPWTFGQGTKVEIK
SEQ ID NO:43
anti-CD3 UCHT1v9 V_{H}
EVQLVESGGGLVQPGGSLRLSCAASGYSFTGYTMNWVRQAPGKGLEWVALINPYK
GVSTYNQKFKDRFTISVDKSKNTAYLQMNSLRAEDTAVYYCARSGYYGDSDWYFD
VWGQGTLVTVSS
SEQ ID NO: 44
anti-CD3 V_{L} CDR1
RASSSVSYMN
SEQ ID NO: 45
anti-CD3 V_{L} CDR1
SASSSVSYMN
SEQ ID NO: 46
anti-CD3 VL CDR1
RSSTGAVTTSNYAN
SEQ ID NO:47
anti-CD3 V_{L} CDR1
RASQSVSYMN
SEQ ID NO: 48
anti-CD3 V_{L} CDR2
DTSKVAS
SEQ ID NO: 49
anti-CD3 V_{L} CDR2
DTSKLAS
SEQ ID NO: 50
anti-CD3 V_{L} CDR2
GTNKRAP
SEQ ID NO: 51
anti-CD3 V_{L} CDR3
QQWSSNPLT
SEQ ID NO: 52
anti-CD3 V_{L} CDR3
QQWSSNPFT
SEQ ID NO: 53
anti-CD3 V_{L} CDR3
ALWYSNLWV
SEQ ID NO: 54
anti-CD3 V_{H} CDR1
RYTMH
SEQ ID NO: 55
anti-CD3 V_{H} CDR1
TYAMN
SEQ ID NO: 56
anti-CD3 V_{H} CDR2
YINPSRGYTNYNQKFK
SEQ ID NO: 57
anti-CD3 V_{H} CDR2
RIRSKYNNYATYYADSVKD
SEQ ID NO: 58
anti-CD3 V_{H} CDR2
YINPSRGYTNYADSVKG
SEQ ID NO: 59
anti-CD3 V_{H} CDR3
YYDDHYCLDY
SEQ ID NO: 60
anti-CD3 V_{H} CDR3
HGNFGNSYVSWFAY
SEQ ID NO: 61
anti-CD3 L2K-07 V_{L}
DIQLTQSPAIMSASPGEKVTMTCRASSSVSYMNWYQQKSGTSPKRWIYDTSKVASG
VPYRFSGSGSGTSYSLTISSMEAEDAATYYCQQWSSNPLTFGAGTKLELK
SEQ ID NO: 62
anti-CD3 L2K-07 V_{H}
DIKLQQSGAELARPGASVKMSCKTSGYTFTRYTMHWVKQRPGQGLEWIGYINPSRG
YTNYNQKFKDKATLTTDKSSSTAYMQLSSLTSEDSAVYYCARYYDDHYCLDYWGQ
GTTLTVSS
SEQ ID NO: 63
anti-CD3 OKT3 V_{L}
QIVLTQSPAIMSASPGEKVTMTCSASSSVSYMNWYQQKSGTSPKRWIYDTSKLASGV
PAHFRGSGSGTSYSLTISGMEAEDAATYYCQQWSSNPFTFGSGTKLEIN
SEQ ID NO: 64
anti-CD3 OKT3 V_{H}
QVQLQQSGAELARPGASVKMSCKASGYTFTRYTMHWVKQRPGQGLEWIGYINPSR
GYTNYNQKFKDKATLTTDKSSSTAYMQLSSLTSEDSAVYYCARYYDDHYCLDYWG
QGTTLTVSS
SEQ ID NO: 65
anti-CD3 hXR32 V_{L}
QAVVTQEPSLTVSPGGTVTLTCRSSTGAVTTSNYANWVQQKPGQAPRGLIGGTNKR
APWTPARFSGSLLGGKAALTITGAQAEDEADYYCALWYSNLWVFGGGTKLTVL
SEQ ID NO: 66
anti-CD3 hXR32 V_{H}
EVOLVESGGGLVQPGGSLRLSCAASGFTFNTYAMNWVRQAPGKGLEWVARIRSKY
NNYATYYADSVKDRFTISRDDSKNSLYLQMNSLKTEDTAVYYCVRHGNFGNSYVS
WFAYWGQGTLVTVSS
SEQ ID NO: 67
anti-CD3 diL2K V_{L}
DIVLTQSPATLSLSPGERATLSCRASQSVSYMNWYQQKPGKAPKRWIYDTSKVASG
VPARFSGSGSGTDYSLTINSLEAEDAATYYCQQWSSNPLTFGGGTKVEIK
SEQ ID NO: 68
anti-CD3 diL2K V_{H}
DVQLVQSGAEVKKPGASVKVSCKASGYTFTRYTMHWVRQAPGQGLEWIGYINPSR
GYTNYADSVKGRFTITTDKSTSTAYMELSSLRSEDTATYYCARYYDDHYCLDYWG
QGTTVTVSS
SEQ ID NO: 69
polypeptide linker
GGSGGSGGSGGSGGVD

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A bispecific molecule comprising:
a first polypeptide comprising a first antigen binding domain that can bind a T cell receptor β chain variable (TRBV) polypeptide; and
a second polypeptide comprising a second antigen binding domain that can bind a T cell co-receptor polypeptide.

2. The bispecific molecule of claim 1, wherein said first polypeptide is selected from the group consisting of a single-chain variable fragment (scFv), an antigen-binding fragment (Fab), a F(ab′)2 fragment, and biologically active fragments thereof.

3. The bispecific molecule of claim 1, wherein said TRBV polypeptide is selected from the group consisting of a TRBV2 polypeptide, a TRBV3-1 polypeptide, a TRBV4-1 polypeptide, a TRBV4-2 polypeptide, a TRBV4-3 polypeptide, a TRBV5-1 polypeptide, a TRBV5-4 polypeptide, a TRBV5-5 polypeptide, a TRBV5-6 polypeptide, a TRBV5-8 polypeptide, a TRBV6-1 polypeptide, a TRBV6-2 polypeptide, a TRBV6-3 polypeptide, a TRBV6-4 polypeptide, a TRBV6-5 polypeptide, a TRBV6-6 polypeptide, a TRBV6-8 polypeptide, a TRBV6-9 polypeptide, a TRBV7-2 polypeptide, a TRBV7-3 polypeptide, a TRBV7-4 polypeptide, a TRBV7-6 polypeptide, a TRBV7-7 polypeptide, a TRBV7-8 polypeptide, a TRBV7-9 polypeptide, a TRBV9 polypeptide, a TRBV10-1 polypeptide, a TRBV10-2 polypeptide, a TRBV10-3 polypeptide, a TRBV11-1 polypeptide, a TRBV11-2 polypeptide, a TRBV11-3 polypeptide, a TRBV12-2 polypeptide, a TRBV12-3 polypeptide, a TRBV12-4 polypeptide, a TRBV12-5 polypeptide, a TRBV13 polypeptide, a TRBV14 polypeptide, a TRBV15 polypeptide, a TRBV16 polypeptide, a TRBV18 polypeptide, a TRBV19 polypeptide, a TRBV20-1 polypeptide, a TRBV24-1 polypeptide, a TRBV25-1 polypeptide, a TRBV27 TRBV28 polypeptide, a TRBV29-1 polypeptide, and a TRBV30 polypeptide.

4. The bispecific molecule of claim 3, wherein said TRBV polypeptide is said TRBV5-5 polypeptide.

5. The bispecific molecule of claim 4, wherein said first antigen binding domain that can bind to said TRBV5-5 polypeptide comprises:
a light chain including a V_{L} CDR1 having an amino acid sequence set forth in SEQ ID NO:1, a V_{L} CDR2 having an amino acid sequence set forth in SEQ ID NO:2, and a V_{L} CDR3 having an amino acid sequence set forth in SEQ ID NO:3; and
a heavy chain including a V_{H} CDR1 having an amino acid sequence set forth in SEQ ID NO:4, a V_{H} CDR2 having an amino acid sequence set forth in SEQ ID NO:5, and a V_{H} CDR3 having an amino acid sequence set forth in SEQ ID NO:6.

6. The bispecific molecule of claim 5, wherein said light chain comprises an amino acid sequence set forth in SEQ ID NO:7, and wherein said heavy chain comprises an amino acid sequence set forth in SEQ ID NO:8.

7. The bispecific molecule of claim 5, wherein said light chain comprises an amino acid sequence set forth in SEQ ID NO:38, and wherein said heavy chain comprises an amino acid sequence set forth in SEQ ID NO:39.

8. The bispecific molecule of claim 3, wherein said TRBV polypeptide is said TRBV12 polypeptide.

9. The bispecific molecule of claim 8, wherein said first antigen binding domain that can bind to said TRBV12 polypeptide comprises:
a light chain including a V_{L} CDR1 having an amino acid sequence set forth in SEQ ID NO:9, a V_{L} CDR2 having an amino acid sequence set forth in SEQ ID NO:10, and a V_{L} CDR3 having an amino acid sequence set forth in SEQ ID NO:11; and
a heavy chain including a V_{H} CDR1 having an amino acid sequence set forth in SEQ ID NO:12, a V_{H} CDR2 having an amino acid sequence set forth in SEQ ID NO:13, and a V_{H} CDR3 having an amino acid sequence set forth in SEQ ID NO:14.

10. The bispecific molecule of claim 9, wherein said light chain comprises an amino acid sequence set forth in SEQ ID NO:15, and wherein said heavy chain comprises an amino acid sequence set forth in SEQ ID NO:16.

11. The bispecific molecule of claim 9, wherein said light chain comprises an amino acid sequence set forth in SEQ ID NO:40, and wherein said heavy chain comprises an amino acid sequence set forth in SEQ ID NO:41.

12. The bispecific molecule of claim 1, wherein said second polypeptide is selected from the group consisting of a single-chain variable fragment (scFv), an antigen-binding fragment (Fab), a F(ab′)2 fragment, and biologically active fragments thereof.

13. The bispecific molecule of claim 1, wherein said T cell co-receptor polypeptide is a cluster of differentiation 3 (CD3) polypeptide.

14. The bispecific molecule of claim 13, wherein said second antigen binding domain that can bind to said CD3 polypeptide comprises:
a light chain including a V_{L} CDR1 having an amino acid sequence set forth in SEQ ID NO:17, a V_{L} CDR2 having an amino acid sequence set forth in SEQ ID NO:18, and a V_{L} CDR3 having an amino acid sequence set forth in SEQ ID NO:19; and
a heavy chain including a V_{H} CDR1 having an amino acid sequence set forth in SEQ ID NO:20, a V_{H} CDR2 having an amino acid sequence set forth in SEQ ID NO:21, and a V_{H} CDR3 having an amino acid sequence set forth in SEQ ID NO:22.

15. The bispecific molecule of claim 14, wherein said light chain comprises an amino acid sequence set forth in SEQ ID NO:23, and wherein said heavy chain comprises an amino acid sequence set forth in SEQ ID NO:24.

16. A method for treating a mammal having a T cell cancer, said method comprising administering to said mammal a bispecific molecule comprising:
a first polypeptide comprising a first antigen binding domain that can bind a T cell receptor β chain variable (TRBV) polypeptide; and
a second polypeptide comprising a second antigen binding domain that can bind a T cell co-receptor polypeptide.

17. The method of claim 16, wherein said mammal is a human.

18. The method of claim 16, wherein said T cell cancer is a clonal T cell cancer.

19. The method of claim 16, wherein said T cell cancer is selected from the group consisting of acute lymphoblastic leukemia (ALL), peripheral T cell lymphomas (PTCL), angioimmunoblastic T cell lymphomas (AITL), T cell prolymphocytic leukemia (T-PLL), adult T cell leukemia/lymphoma (ATLL), Enteropathy-associated T-cell lymphoma (EATL), monomorphic epitheliotropic intestinal T-cell lymphoma (MEITL), follicular T-cell lymphoma (FTCL), nodal peripheral T-cell lymphoma (nodal PTCL), cutaneous T cell lymphomas (CTCL), anaplastic large cell lymphoma (ALCL), T-cell large granular lymphocytic leukemia (T-LGL), extra nodal NK/T-Cell lymphoma (NKTL), and hepatosplenic T-cell lymphoma.

20. The method of claim 16, wherein said cancer cells within said mammal are reduced by at least 95 percent.

21. The method of claim 16, wherein said method is effective to improve survival of said mammal.

22. The method of claim 21, wherein said survival of said mammal is improved by at least 37.5 percent.

23. A method for treating a mammal having celiac disease, said method comprising administering to said mammal a bispecific molecule comprising:
a first polypeptide comprising a first antigen binding domain that can bind a T cell receptor β chain variable (TRBV) polypeptide; and
a second polypeptide comprising a second antigen binding domain that can bind a T cell co-receptor polypeptide.

24. The method of claim 23, wherein said mammal is a human.

25. The method of claim 23, wherein said TRBV polypeptide is selected from the group consisting of TRBV4, TRBV6, TRBV7, TRBV9, TRBV20 and TRBV29, and wherein said T cell co-receptor polypeptide is a CD3 polypeptide.

26. The method of claim 23, wherein said TRBV polypeptide is selected from the group consisting of TRBV6-1, TRBV7-2, TRBV9-1, TRBV20-1 and TRBV29-1, and wherein said T cell co-receptor polypeptide is a CD3 polypeptide.


SEQUENCE LISTING (69 sequences)

SEQ ID NO: 1 - protein, 12 aa, artificial
  anti-TRBV5-5 VL CDR1
CSASQGISNYLN

SEQ ID NO: 2 - protein, 8 aa, artificial
  anti-TRBV5-5 VL CDR2
TSSLHSGV

SEQ ID NO: 3 - protein, 9 aa, artificial
  anti-TRBV5-5 VL CDR3
QQYSKLPRT

SEQ ID NO: 4 - protein, 5 aa, artificial
  anti-TRBV5-5 VH CDR1
AYGVN

SEQ ID NO: 5 - protein, 13 aa, artificial
  anti-TRBV5-5 VH CDR2
WGDGNTDYNSALK

SEQ ID NO: 6 - protein, 8 aa, artificial
  anti-TRBV5-5 VH CDR3
ATLYAMDY

SEQ ID NO: 7 - protein, 107 aa, artificial
  anti-TRBV5-5 VL
DIQMTQTTSSLSASLGDRVTITCSASQGISNYLNWYQQKPDGTVKLLIYYTSSLHSGVPSRFSGSGSGTDYSLTISNLEPEDIATYYCQQYSKLPRTFGGGTKVEIK

SEQ ID NO: 8 - protein, 120 aa, artificial
  anti-TRBV5-5 VH
QVQLKESGPGLVAPSQSLSITCTVSGFSLTAYGVNWVRQPPGKGLEWLGMIWGDGNTDYNSALKSRLSISKDNSKSQVFLKMNSLQTDDTARYYCARDRVTATLYAMDYWGQGTSVTVSS

SEQ ID NO: 9 - protein, 12 aa, artificial
  anti-TRBV12 VL CDR1
CRASSSVNYIYW

SEQ ID NO: 10 - protein, 10 aa, artificial
  anti-TRBV12 VL CDR2
YTSNLAPGVP

SEQ ID NO: 11 - protein, 9 aa, artificial
  anti-TRBV12 VL CDR3
QQFTSSPFT

SEQ ID NO: 12 - protein, 5 aa, artificial
  anti-TRBV12 VH CDR1
NFGMH

SEQ ID NO: 13 - protein, 17 aa, artificial
  anti-TRBV12 VH CDR2
YISSGSSTIYYADTLKG

SEQ ID NO: 14 - protein, 8 aa, artificial
  anti-TRBV12 VH CDR3
RGEGAMDY

SEQ ID NO: 15 - protein, 106 aa, artificial
  anti-TRBV12 VL
ENVLTQSPAIMSASLGEKVTMSCRASSSVNYIYWYQQKSDASPKLWIYYTSNLAPGVPTRFSGSGSGNSYSLTISSMEGEDAATYYCQQFTSSPFTFGQGTKLEIK

SEQ ID NO: 16 - protein, 117 aa, artificial
  anti-TRBV12 VH
DVQLVESGGGLVQPGGSRKLSCAASGFTFSNFGMHWVRQAPDKGLEWVAYISSGSSTIYYADTLKGRFTISRDNPKNTLFLQMTSLRSEDTAMYYCARRGEGAMDYWGQGTSVTVSS

SEQ ID NO: 17 - protein, 11 aa, artificial
  anti-CD3 VL CDR1
RASQDIRNYLN

SEQ ID NO: 18 - protein, 8 aa, artificial
  anti-CD3 VL CDR2
  MISC_FEATURE (1)..(1): Y at position 1 is optional
YYTSRLHS

SEQ ID NO: 19 - protein, 9 aa, artificial
  anti-CD3 VL CDR3
QQGNTLPWT

SEQ ID NO: 20 - protein, 5 aa, artificial
  anti-CD3 VH CDR1
GYTMN

SEQ ID NO: 21 - protein, 17 aa, artificial
  anti-CD3 VH CDR2
LINPYKGVSTYNQKFKD

SEQ ID NO: 22 - protein, 13 aa, artificial
  anti-CD3 VH CDR3
SGYYGDSDWYFDV

SEQ ID NO: 23 - protein, 107 aa, artificial
  anti-CD3 UCHT1 VL
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIK

SEQ ID NO: 24 - protein, 122 aa, artificial
  anti-CD3 UCHT1 VH
EVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGAGTTVTVSS

SEQ ID NO: 25 - protein, 5 aa, artificial
  polypeptide linker
GGGGS

SEQ ID NO: 26 - protein, 15 aa, artificial
  polypeptide linker
GGGGSGGGGSGGGGS

SEQ ID NO: 27 - protein, 113 aa, artificial
  anti-TRBC1 VL
DVVMTQSPLSLPVSLGDQASISCRSSQRLVHSNGNTYLHWYLQKPGQSPKLLIYRVSNRFPGVPDRFSGSGSGTDFTLKISRVEAEDLGIYFCSQSTHVPYTFGGGTKLEIKR

SEQ ID NO: 28 - protein, 124 aa, artificial
  anti-TRBC1 VH
EVRLQQSGPDLIKPGASVKMSCKASGYTFTGYVMHWVKQRPGQGLEWIGFINPYNDDIQSNERFRGKATLTSDKSSTTAYMELSSLTSEDSAVYYCARGAGYNFDGAYRFFDFWGQGTTLTVSS

SEQ ID NO: 29 - protein, 111 aa, artificial
  anti-CD19 VL
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 30 - protein, 124 aa, artificial
  anti-CD19 VH
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 31 - DNA, 20 bp, artificial
  nucleic acid encoding crRNA targeting the TRA constant region
agagtctctcagctggtaca

SEQ ID NO: 32 - DNA, 20 bp, artificial
  nucleic acid encoding crRNA targeting the TRB constant region
agaaggtggccgagaccctc

SEQ ID NO: 33 - DNA, 20 bp, artificial
  PCR primer
gcctaagttggggagaccac

SEQ ID NO: 34 - DNA, 20 bp, artificial
  PCR primer
gaagcaaggaaacagcctgc

SEQ ID NO: 35 - DNA, 22 bp, artificial
  PCR primer
tcgctgtgtttgagccatcaga

SEQ ID NO: 36 - DNA, 22 bp, artificial
  PCR primer
atgaaccacaggtgcccaattc

SEQ ID NO: 37 - DNA, 20 bp, artificial
  nucleic acid encoding sgRNA targeting CD19
cgaggaacctctagtggtga

SEQ ID NO: 38 - protein, 107 aa, artificial
  anti-TRBV5-5 VL
  MISC_FEATURE (79)..(79): X at residue 78 is a pyrrolysine
DIQMTQSPSSLSASVGDRVTITCSASQGISNYLNWYQQTPGKAPKLLIYYTSSLHSGVPSRFSGSGSGTDYTFTISSLXPEDIATYYCQQYSKLPRTFGQGTKLQIT

SEQ ID NO: 39 - protein, 120 aa, artificial
  anti-TRBV5-5 VH
QVQLQESGPGLVRPSQSLSITCTVSGFSLTAYGVNWVRQPPGRGLEWLGMIWGDGNTDYNSALKSRVTMLKDTSKNQFSLRLSSVTAADTAVYYCARDRVTATLYAMDYWGQGSLVTVSS

SEQ ID NO: 40 - protein, 106 aa, artificial
  anti-TRBV12 VL
DIQMTTQSPSSLSASVGDRVTITCRASSSVNYIYWYQQTPGKAPKLLIYYTSNLAPGVPSRFSGSGSGTDYTFTISSLQPEDITYYCQQFTSSPFTFGSGTKLQIT

SEQ ID NO: 41 - protein, 117 aa, artificial
  anti-TRBV12 VH
EVQLVESGGGVVQPGGSRKLSCSSSGFTFSNFGMHWVRQAPGKGLEWVAYISSGSSTIYYADTLKGRFTISRDNSKNTLFLQMDSLRPEDTGVYFCARRGEGAMDYWGQGTSVTVSS

SEQ ID NO: 42 - protein, 107 aa, artificial [sequence omitted]

SEQ ID NO: 43 - protein, 122 aa, artificial [sequence omitted]

SEQ ID NO: 44 - protein, 10 aa, artificial
  anti-CD3 VL CDR1
RASSSVSYMN

SEQ ID NO: 45 - protein, 10 aa, artificial
  anti-CD3 VL CDR1
SASSSVSYMN

SEQ ID NO: 46 - protein, 14 aa, artificial
  anti-CD3 VL CDR1
RSSTGAVTTSNYAN

SEQ ID NO: 47 - protein, 10 aa, artificial
  anti-CD3 VL CDR1
RASQSVSYMN

SEQ ID NO: 48 - protein, 7 aa, artificial
  anti-CD3 VL CDR2
DTSKVAS

SEQ ID NO: 49 - protein, 7 aa, artificial
  anti-CD3 VL CDR2
DTSKLAS

SEQ ID NO: 50 - protein, 7 aa, artificial
  anti-CD3 VL CDR2
GTNKRAP

SEQ ID NO: 51 - protein, 9 aa, artificial
  anti-CD3 VL CDR3
QQWSSNPLT

SEQ ID NO: 52 - protein, 9 aa, artificial
  anti-CD3 VL CDR3
QQWSSNPFT

SEQ ID NO: 53 - protein, 9 aa, artificial
  anti-CD3 VL CDR3
ALWYSNLWV

SEQ ID NO: 54 - protein, 5 aa, artificial
  anti-CD3 VH CDR1
RYTMH

SEQ ID NO: 55 - protein, 5 aa, artificial
  anti-CD3 VH CDR1
TYAMN

SEQ ID NO: 56 - protein, 16 aa, artificial
  anti-CD3 VH CDR2
YINPSRGYTNYNQKFK

SEQ ID NO: 57 - protein, 19 aa, artificial
  anti-CD3 VH CDR2
RIRSKYNNYATYYADSVKD

SEQ ID NO: 58 - protein, 17 aa, artificial
  anti-CD3 VH CDR2
YINPSRGYTNYADSVKG

SEQ ID NO: 59 - protein, 10 aa, artificial
  anti-CD3 VH CDR3
YYDDHYCLDY

SEQ ID NO: 60 - protein, 14 aa, artificial
  anti-CD3 VH CDR3
HGNFGNSYVSWFAY

SEQ ID NO: 61 - protein, 106 aa, artificial
  anti-CD3 L2K-07 VL
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 62 - protein, 119 aa, artificial
  anti-CD3 L2K-07 VH
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 63 - protein, 106 aa, artificial [sequence omitted]

SEQ ID NO: 64 - protein, 119 aa, artificial [sequence omitted]

SEQ ID NO: 65 - protein, 109 aa, artificial
  anti-CD3 hXR32 VL
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVL

SEQ ID NO: 66 - protein, 125 aa, artificial
  anti-CD3 hXR32 VH
EVQLVESGGGLVQPGGSLRLSCAASGFTFNTYAMNWVRQAPGKGLEWVARIRSKYNNYATYYADSVKDRFTISRDDSKNSLYLQMNSLKTEDTAVYYCVRHGNFGNSYVSWFAYWGQGTLVTVSS

SEQ ID NO: 67 - protein, 106 aa, artificial
  anti-CD3 diL2K VL
DIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 68 - protein, 119 aa, artificial
  anti-CD3 diL2K VH
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSS

SEQ ID NO: 69 - protein, 16 aa, artificial
  polypeptide linker
GGSGGSGGSGGSGGVD